(19) United States
(12) Patent Application Publication
(10) Pub. No.: US 2006/0228706 A1
(43) Pub. Date: Oct. 12, 2006

(54) Methods of diagnosing and treating diabetes and insulin resistance

(75) Inventors: Allan Bernard, San Francisco, CA (US); Brian Lavan, San Francisco, CA (US); Shonna Moodie, San Francisco, CA (US); Steve Waters, San Ramon, CA (US); Chi-Wai Wong, Hayward, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Metabolex, Inc., Hayward, CA (US)

(21) Appl. No.: 10/516,803

(22) PCT Filed: Jun. 4, 2003
(86) PCT No.: PCT/US03/17941

§ 371 Date: Dec. 2, 2004

Related U.S. Application Data
(60) Provisional application No. 60/386,113, filed on Jun. 4, 2002.
(60) Provisional application No. 60/385,996, filed on Jun. 4, 2002.
(60) Provisional application No. 60/386,935, filed on Jun. 6, 2002.
(60) Provisional application No. 60/387,038, filed on Jun. 6, 2002.
(60) Provisional application No. 60/386,956, filed on Jun. 6, 2002.
(60) Provisional application No. 60/386,958, filed on Jun. 6, 2002.
(60) Provisional application No. 60/386,812, filed on Jun. 6, 2002.

(51) Int. Cl. C12Q 1/68; G01N 33/567; C07H 21/04; C12P 21/06; C07K 14/72; C07K 16/28

(52) U.S. Cl. 435/6; 435/7.2; 435/69.1; 435/320.1; 435/325; 530/350; 530/388.22; 536/23.5

(57) ABSTRACT

The present invention provides compositions and methods for diagnosing and treating diabetes and insulin resistance. In particular, the invention provides methods of identifying modulators of the polynucleotides or polypeptides of the invention and using those modulators to treat diabetes, as well as methods of diagnosing diabetes by measuring the levels of the polynucleotides or polypeptides of the invention in a patient.

### CROSS REFERENCE TO RELATED APPLICATIONS

This application claims the benefit of priority to U.S. provisional application No. 60/386,113, filed Jun. 4, 2002; U.S. provisional application No. 60/386,935, filed Jun. 6, 2002; U.S. provisional application No. 60/387,038, filed Jun. 6, 2002; U.S. provisional application No. 60/386,956, filed Jun. 6, 2003; U.S. provisional application No. 60/386,958, filed Jun. 6, 2002; U.S. provisional application No. 60/386,812, filed Jun. 6, 2002; and U.S. provisional application No. 60/385,996, filed Jun. 4, 2002, each of which applications is herein incorporated by reference.

### BACKGROUND OF THE INVENTION

Diabetes mellitus can be divided into two clinical syndromes, Type 1 and Type 2 diabetes mellitus. Type 1, or insulin-dependent diabetes mellitus (IDDM), is a chronic autoimmune disease characterized by the extensive loss of beta cells in the pancreatic Islets of Langerhans, which produce insulin. As these cells are progressively destroyed, the amount of secreted insulin decreases, eventually leading to hyperglycemia (abnormally high level of glucose in the blood) when the amount of secreted insulin drops below the level required for euglycemia (normal blood glucose level). Although the exact trigger for this immune response is not known, patients with IDDM have high levels of antibodies against proteins expressed in pancreatic beta cells. However, not all patients with high levels of these antibodies develop IDDM.

Type 2 diabetes (also referred to as non-insulin dependent diabetes mellitus (NIDDM)) develops when muscle, fat and liver cells fail to respond normally to insulin. This failure to respond (called insulin resistance) may be due to reduced numbers of insulin receptors on these cells, or a dysfunction of signaling pathways within the cells, or both. The beta cells initially compensate for this insulin resistance by increasing insulin output. Over time, these cells become unable to produce enough insulin to maintain normal glucose levels, indicating progression to Type 2 diabetes.

Type 2 diabetes is brought on by a combination of genetic and acquired risk factors—including a high-fat diet, lack of exercise, and aging. Worldwide, Type 2 diabetes has become an epidemic, driven by increases in obesity and a sedentary lifestyle, widespread adoption of western dietary habits, and the general aging of the population in many countries. In 1985, an estimated 30 million people worldwide had diabetes—by 2000, this figure had increased 5-fold, to an estimated 154 million people. The number of people with diabetes is expected to double between now and 2025, to about 300 million.

Type 2 diabetes is a complex disease characterized by defects in glucose and lipid metabolism. Typically there are perturbations in many metabolic parameters including increases in fasting plasma glucose levels, free fatty acid levels and triglyceride levels, as well as a decrease in the ratio of HDL/LDL. As discussed above, one of the principal underlying causes of diabetes is thought to be an increase in insulin resistance in peripheral tissues, principally muscle and fat. The present invention addresses this and other problems.

### BRIEF SUMMARY OF THE INVENTION

The present invention provides methods for identifying an agent for treating a diabetic or pre-diabetic individual. In some embodiments, the methods comprise the steps of: (i) contacting an agent to a mixture comprising a polypeptide encoded by a polynucleotide that hybridizes under stringent conditions to a nucleic acid encoding SEQ ID NO:2, SEQ ID NO:8, SEQ ID NO:12, SEQ ID NO:16, SEQ ID NO:22, SEQ ID NO:26, SEQ ID NO:28, or SEQ ID NO:32; and (ii) selecting an agent that modulates the expression or activity of the polypeptide or that binds to the polypeptide, thereby identifying an agent for treating a diabetic or pre-diabetic individual. In some embodiments, the methods further comprise selecting an agent that modulates insulin sensitivity.

In some embodiments, step (ii) comprises selecting an agent that modulates expression of the polypeptide. In some embodiments, step (ii) comprises selecting an agent that modulates the activity of the polypeptide. In some embodiments, step (ii) comprises selecting an agent that specifically binds to the polypeptide. In some embodiments, the polypeptide is expressed in a cell and the cell is contacted with the agent. In some embodiments, the polypeptide comprises SEQ ID NO:2, SEQ ID NO:8, SEQ ID NO:12, SEQ ID NO:16, SEQ ID NO:22, SEQ ID NO:26, SEQ ID NO:28, or SEQ ID NO:32. In other embodiments, the polypeptide comprises SEQ ID NO:4, SEQ ID NO:6, SEQ ID NO:10, SEQ ID NO:14, SEQ ID NO:18, SEQ ID NO:20, SEQ ID NO:24, SEQ ID NO:30, or SEQ ID NO:34.

The present invention also provides methods of treating a diabetic or pre-diabetic animal. In some embodiments, the methods comprise administering to the animal a therapeutically effective amount of an agent identified as described above. In some embodiments, the agent is an antibody. In some embodiments, the antibody is a monoclonal antibody. In some embodiments, the animal is a human.

The present invention also provides methods of introducing an expression cassette into a cell. In some emodiments, the methods comprise introducing into the cell an expression cassette comprising a promoter operably linked to a polynucleotide encoding a polypeptide, wherein the polynucleotide hybridizes under stringent conditions to a nucleic acid encoding SEQ ID NO:2, SEQ ID NO:8, SEQ ID NO:12, SEQ ID NO:16, SEQ ID NO:22, SEQ ID NO:26, SEQ ID NO:28, or SEQ ID NO:32.

In some embodiments, th polypeptide comprises SEQ ID NO:2, SEQ ID NO:8, SEQ ID NO:12, SEQ ID NO:16, SEQ ID NO:22, SEQ ID NO:26, SEQ ID NO:28, or SEQ ID NO:32. In other embodiments, the polypeptide comprises SEQ ID NO:4, SEQ ID NO:6, SEQ ID NO:10, SEQ ID NO:14, SEQ ID NO:18, SEQ ID NO:20, SEQ ID NO:24, SEQ ID NO:30, or SEQ ID NO:34. In some embodiments, the cell is selected from the group consisting of an adipocyte and a skeletal muscle cell.

In some embodiments, the methods further comprising introducing the cell into a human. In some embodiments, the human is diabetic. In some embodiments, the human is prediabetic. In some embodiments, the cell is from the human.

The present invention also provides methods of diagnosing an individual who has Type 2 diabetes or is prediabetic. In some embodiments, the method comprises, detecting in a sample from the individual the level of a polypeptide or the level of a polynucleotide encoding the polypeptide, wherein the polynucleotide hybridizes under stringent conditions to a nucleic acid encoding an amino acid sequence selected from the group consisting of SEQ ID NO:2, SEQ ID NO:8, SEQ ID NO:12, SEQ ID NO:16, SEQ ID NO:22, SEQ ID NO:26, SEQ ID NO:28, and SEQ ID NO:32; wherein a modulated level of the polypeptide or polynucleotide in the sample compared to a level of the polypeptide or polynucleotide in either a lean individual or a previous sample from the individual indicates that the individual 30 is diabetic or prediabetic. In some embodiments, the amino acid sequence comprises SEQ ID NO:2, SEQ ID NO:8, SEQ ID NO:12, SEQ ID NO:16, SEQ ID NO:22, SEQ ID NO:26, SEQ ID NO:28, or SEQ ID NO:32.

In some embodiments, the detecting step comprises contacting the sample with an antibody that specifically binds to the polypeptide.

In some embodiments, the detecting step comprises quantifying mRNA encoding the polypeptide. In some embodiments, the mRNA is reverse transcribed and amplified in a polymerase chain reaction.

In some embodiments, the sample is a blood, urine or tissue sample.

The present invention also provides isolated nucleic acids that hybridize under stringent conditions to a polynucleotide encoding a polypeptide having an amino acid sequence selected from the group consisting of SEQ ID NO:2, SEQ ID NO:8, SEQ ID NO:12, SEQ ID NO:16, SEQ ID NO:22, SEQ ID NO:26, SEQ ID NO:28, and SEQ ID NO:32.

In some embodiments, the nucleic acid comprises SEQ ID NO:1, SEQ ID NO:7, SEQ ID NO:11, SEQ ID NO:15, SEQ ID NO:21, SEQ ID NO:25, SEQ ID NO:27, or SEQ ID NO:31. In some embodiments, the nucleic acid encodes SEQ ID NO:2, SEQ ID NO:8, SEQ ID NO:12, SEQ ID NO:16, SEQ ID NO:22, SEQ ID NO:26, SEQ ID NO:28, or SEQ ID NO:32.

The present invention also provides an expression cassette comprising comprising a heterologous promoter operably linked to a polynucleotide that hybridizes under stringent conditions to a nucleic acid encoding a polypeptide having an amino acid sequence selected from the group consisting of SEQ ID NO:2, SEQ ID NO:8, SEQ ID NO:12, SEQ ID NO:16, SEQ ID NO:22, SEQ ID NO:26, SEQ ID NO:28, and SEQ ID NO:32. In some embodiments, the polynucleotide encodes a polypeptide that has an amino acid sequence selected from the group consisting of SEQ ID NO:2, SEQ ID NO:8, SEQ ID NO:12, SEQ ID NO:16, SEQ ID NO:22, SEQ ID NO:26, SEQ ID NO:28, and SEQ ID NO:32.

The present invention also provides host cells transfected with a polynucleotide comprising the expression cassette. In some embodiments, the host cell is a human cell. In some embodiments, the host cell is a bacterium.

The present invention also provides isolated polypeptides comprising an amino acid sequence at least 70% identical to SEQ ID NO:2, SEQ ID NO:8, SEQ ID NO:12, SEQ ID NO:16, SEQ ID NO:22, SEQ ID NO:26, SEQ ID NO:28, or SEQ ID NO:32. In some embodiments, the polypeptide comprises SEQ ID NO:2, SEQ ID NO:8, SEQ ID NO:12, SEQ ID NO:16, SEQ ID NO:22, SEQ ID NO:26, SEQ ID NO:28, or SEQ ID NO:32.

### DEFINITIONS

“Insulin sensitivity” refers to the ability of a cell or tissue to respond to insulin. Responses include, e.g., glucose uptake of a cell or tissue in response to insulin stimulation. Sensitivity can be determined at an organismal, tissue or cellular level. For example, blood or urine glucose levels following a glucose tolerance test are indicative of insulin sensitivity. Other methods of measuring insulin sensitivity include, e.g., measuring glucose uptake (see, e.g., Garcia de Herreros, A., and Birnbaum, M. J. J. Biol. Chem. 264, 19994-19999 (1989); Klip, A., Li, G., and Logan, W. J. Am. J. Physiol. 247, E291-296 (1984)), measuring the glucose infusion rate (GINF) into tissue such as the skeletal muscle (see, e.g., Ludvik et al., J. Clin. Invest. 100:2354 (1997); Frias et al., Diabetes Care 23:64, (2000)) and measuring sensitivity of GLUT4 translocation (e.g., as described herein) in response to insulin.

“Activity” of a polypeptide of the invention refers to structural, regulatory, or biochemical functions of a polypeptide in its native cell or tissue. Examples of activity of a polypeptide include both direct activities and indirect activities. Exemplary direct activities are the result of firect interaction with the polypeptide, , e.g., enzymatic activity, ligand binding, production or depletion of second messengers (e.g., cAMP, cGMP, IP₃, DAG, or Ca²⁺), ion flux, phosphorylation levels, transcription levels, and the like. Exemplary indirect activities are observed as a change in phenotype or response in a cell or tissue to a polypeptide's directed activity, e.g., modulating insulin sensitivity of a cell as a result of the interaction of the polypeptide with other cellular or tissue components.

“Predisposition for diabetes” occurs in a person when the person is at high risk for developing diabetes. A number of risk factors are known to those of skill in the art and include: genetic factors (e.g., carrying alleles that result in a higher occurrence of diabetes than in the average population or having parents or siblings with diabetes); overweight (e.g., body mass index (BMI) greater or equal to 25 kg/m²); habitual physical inactivity, race/ethnicity (e.g., African-American, Hispanic-American, Native Americans, Asian-Americans, Pacific Islanders); previously identified impaired fasting glucose or impaired glucose tolerance, hypertension (e.g., greater or equal to 140/90 mmHg in adults); HDL cholesterol less than or equal to 35 mg/dl; triglyceride levels greater or equal to 250 mg/dl; a history of gestational diabetes or delivery of a baby over nine pounds; and/or polycystic ovary syndrome. See, e.g., “Report of the Expert Committee on the Diagnosis and Classification of Diabetes Mellitus” and “Screening for Diabetes” Diabetes Care 25(1): S5-S24 (2002).

A “lean individual,” when used to compare with a sample from a patient, refers to an adult with a fasting blood glucose level less than 110 mg/dl or a 2 hour PG reading of 140 mg/dl. “Fasting” refers to no caloric intake for at least 8 hours. A “2 hour PG” refers to the level of blood glucose after challenging a patient to a glucose load containing the equivalent of 75 g anhydrous glucose dissolved in water. The overall test is generally referred to as an oral glucose tolerance test (OGTT). See, e.g., Diabetes Care, Supplement 2002, American Diabetes Association: Clinical Practice Recommendations 2002. The level of a polypeptide in a lean individual can be a reading from a single individual, but is typically a statistically relevant average from a group of lean individuals. The level of a polypeptide in a lean individual can be represented by a value, for example in a computer program.

A “pre-diabetic individual,” when used to compare with a sample from a patient, refers to an adult with a fasting blood glucose level greater than 110 mg/dl but less than 126 mg/dl or a 2 hour PG reading of greater than 140 mg/dl but less than 200mg/dl. A “diabetic individual,” when used to compare with a sample from a patient, refers to an adult with a fasting blood glucose level greater than 126 mg/dl or a 2 hour PG reading of greater than 200 mg/dl.

A “diabetes-related nucleic acid” or “diabetes-related polynucleotide” (also referred to as a “nucleic acid of the invention” or a “polynucleotide of the invention”) of the invention is a subsequence or full-length polynucleotide sequence of a gene that encodes a polypeptide, whose activity modulates diabetes or insulin sensitivity, or whose presence or absence is indicative of diabetes or altered insulin sensitivity. Exemplary nucleic acids of the invention include those sequences substantially identical to a sequence disclosed herein, e.g., SEQ ID NO:1, SEQ ID NO:7, SEQ ID NO:11, SEQ ID NO:15, SEQ ID NO:21, SEQ ID NO:25, SEQ ID NO:27, or SEQ ID NO:31; or encode polypeptides substantially identical to polypeptide sequence disclosed herein, e.g., SEQ ID NO:2, SEQ ID NO:8, SEQ ID NO:12, SEQ ID NO:16, SEQ ID NO:22, SEQ ID NO:26, SEQ ID NO:28, or SEQ ID NO:32.

An “agonist” refers to an agent that binds to, stimulates, increases, activates, facilitates, enhances activation, sensitizes or up regulates the activity or expression of a polypeptide of the invention.

An “antagonist” refers to an agent that binds to, partially or totally blocks stimulation, decreases, prevents, delays activation, inactivates, desensitizes, or down regulates the activity or expression of a polypeptide of the invention.

“Antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}—C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies (e.g., single chain Fv).

The terms “peptidomimetic” and “mimetic” refer to a synthetic chemical compound that has substantially the same structural and functional characteristics of the antagonists or agonists of the invention. Peptide analogs are commonly used in the pharmaceutical industry as non-peptide drugs with properties analogous to those of the template peptide. These types of non-peptide compound are termed “peptide mimetics” or “peptidomimetics” (Fauchere, J. Adv. Drug Res. 15:29 (1986); Veber and Freidinger TINS p. 392 (1985); and Evans et al. J. Med. Chem. 30:1229 (1987), which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as a polypeptide exemplified in this application, but have one or more peptide linkages optionally replaced by a linkage selected from the group consisting of, e.g., —CH2NH—, —CH2S—, —CH2-CH2-, —CH═CH— (cis and trans), —COCH2-, —CH(OH)CH2-, and —CH2SO—. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. For example, a mimetic composition is within the scope of the invention if it is capable of carrying out the binding or other activities of an agonist or antagonist of a polypeptide of the invention.

The term “gene” means the segment of DNA involved in producing a polypeptide chain; it includes regions preceding and following the coding region (leader and trailer) as well as intervening sequences (introns) between individual coding segments (exons).

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). The term nucleic acid is used interchangeably with gene, cDNA, and mRNA encoded by a gene.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but which functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide of the invention), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences are substantially identical if two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the following sequence comparison algorithms or by manual alignment and visual inspection. The invention provides polypeptides or polynucleotides that are substantially identical to the polypeptides or polynucleotides, respectively, exemplified herein (e.g., SEQ ID NO:2, SEQ ID NO:8, SEQ ID NO:12, SEQ ID NO:16, SEQ ID NO:22, SEQ ID NO:26, SEQ ID NO:28, or SEQ ID NO:32; or SEQ ID NO:1, SEQ ID NO:7, SEQ ID NO:11, SEQ ID NO:15, SEQ ID NO:21, SEQ ID NO:25, SEQ ID NO:27, or SEQ ID NO:31). This definition also refers to the complement of a test sequence. Optionally, the identity exists over a region that is at least about 50 nucleotides in length, or more preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithmns, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff(1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or 5×SSC, 1% SDS, incubating at 65° C., with wash in 0.2×SSC, and 0.1% SDS at 55° C., 60° C., or 65° C. Such washes can be performed for 5, 15, 30, 60, 120, or more minutes.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1M NaCl, 1% SDS at 37° C., and a wash in 1×SSC at 45° C. Such washes can be performed for 5, 15, 30, 60, 120, or more minutes. A positive hybridization is at least twice background. Those of ordinary skill will readily recognize that alternative hybridization and wash conditions can be utilized to provide conditions of similar stringency.

The phrase “a nucleic acid sequence encoding” refers to a nucleic acid which contains sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons which encode a single amino acid) of the native sequence or sequences that may be introduced to conform with codon preference in a specific host cell.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under-expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “specifically (or selectively) binds to an antibody” or “specifically (or selectively) immunoreactive with”, when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised against a protein having an amino acid sequence encoded by any of the polynucleotides of the invention can be selected to obtain antibodies specifically immunoreactive with that protein and not with other proteins, except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See, Harlow and Lane Antibodies, A Laboratory Manual, Cold Spring Harbor Publications, NY (1988) for a description of immunoassay formats and conditions that can be used to determine specific immunoreactivity. Typically, a specific or selective reaction will be at least twice the background signal or noise and more typically more than 10 to 100 times background.

“Inhibitors,” “activators,” and “modulators” of expression or of activity are used to refer to inhibitory, activating, or modulating molecules, respectively, identified using in vitro and in vivo assays for expression or activity. Modulators encompass e.g., ligands, agonists, antagonists, and their homologs and mimetics. The term “modulator” includes inhibitors and activators. Inhibitors are agents that, e.g., inhibit expression of a polypeptide of the invention or bind to, partially or totally block stimulation, decrease, prevent, delay activation, inactivate, desensitize, or down regulate the activity of a polypeptide of the invention, e.g., antagonists. Activators are agents that, e.g., induce or activate the expression of a polypeptide of the invention or bind to, stimulate, increase, open, activate, facilitate, or enhance activation, sensitize or up regulate the activity of a polypeptide of the invention, e.g., agonists. Modulators include naturally occurring and synthetic ligands, antagonists, agonists, small chemical molecules and the like. Such assays for inhibitors and activators include, e.g., applying putative modulator compounds to cells expressing a polypeptide of the invention and then determining the functional effects on a polypeptide of the invention activity, as described above. Samples or assays comprising a polypeptide of the invention that are treated with a potential activator, inhibitor, or modulator are compared to control samples without the inhibitor, activator, or modulator to examine the extent of effect. Control samples (untreated with modulators) are assigned a relative activity value of 100%. Inhibition of a polypeptide of the invention is achieved when the polypeptide activity value relative to the control is about 80%, optionally 50% or 25, 10%, 5% or 1%. Activation of the polypeptide is achieved when the polypeptide activity value relative to the control is 110%, optionally 150%, optionally 200, 300%, 400%, 500%, or 1000-3000% or more higher.

### DETAILED DESCRIPTION OF THE INVENTION

I. Introduction

The present application demonstrates that, surprisingly, modulated levels of mRNA comprising sequences of the invention occur in muscle tissue of insulin resistant obese, non-diabetic individuals compared to lean, non-diabetic individuals. Insulin resistant obese are generally pre-disposed to become type II diabetics. Therefore, the modulation of the sequences in the study described herein indicates the sequences' involvement in diabetes and pre-diabetes. Without intending to limit the invention to a particular mechanism of action, it is believed that modulation of the expression or activity of the polypeptides of the invention is beneficial in treating diabetic, pre-diabetic or obese insulin resistant, non-diabetic patients. Furthermore, modulated levels of the polypeptides of the invention are indicative of insulin resistance. Thus, the detection of a polypeptide of the invention is useful for diagnosis of diabetes and insulin resistance.

This invention also provides methods of using polypeptides of the invention and modulators of the polypeptides of the invention to diagnose and treat diabetes, pre-diabetes (including insulin resistant individuals) and related metabolic diseases. The present method also provides methods of identifying modulators of expression or activity of the polypeptides of the invention. Such modulators are useful for treating Type 2 diabetes as well as the pathological aspects of diabetes (e.g., insulin resistance).

II. General Recombinant Nucleic Acid Methods for Use with the Invention

In numerous embodiments of the present invention, nucleic acids encoding a polypeptide of the present invention will be isolated and cloned using recombinant methods. Such embodiments are used, e.g., to isolate polynucleotides identical or substantially identical to SEQ ID NO:1, SEQ ID NO:7, SEQ ID NO:11, SEQ ID NO:15, SEQ ID NO:21, SEQ ID NO:25, SEQ ID NO:27, or SEQ ID NO:31 for protein expression or during the generation of variants, derivatives, expression cassettes, or other sequences derived from an polypeptide or polynucleotide of the invention, to monitor gene expression, for the isolation or detection of sequences in different species, for diagnostic purposes in a patient, e.g., to detect mutations in a polypeptide or polynucleotide of the invention or to detect expression levels of nucleic acids or polypeptides. In some embodiments, the sequences encoding the polypeptides of the invention are operably linked to a heterologous promoter. In one embodiment, the nucleic acids of the invention are from any mammal, including, in particular, e.g., a human, a mouse, a rat, etc.

A. General Recombinant Nucleic Acid Methods

This invention relies on routine techniques in the field of recombinant genetics. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

B. Cloning Methods for the Isolation of Nucleotide Sequences Encoding Desired Proteins

In general, the nucleic acids encoding the subject proteins are cloned from DNA sequence libraries that are made to encode cDNA or genomic DNA. The particular sequences can be located by hybridizing with an oligonucleotide probe, the sequence of which can be derived from the sequences disclosed herein, which provide a reference for PCR primers and defines suitable regions for isolating probes pecific for the polypeptides or polynucleotides of the invention. Alternatively, where the sequence is cloned into an expression library, the expressed recombinant protein can be detected immunologically with antisera or purified antibodies made against a polypeptide of interest, including those disclosed herein.

Methods for making and screening genomic and cDNA libraries are well known to those of skill in the art (see, e.g., Gubler and Hoffman Gene 25:263-269 (1983); Benton and Davis Science, 196:180-182 (1977); and Sambrook, supra).

Briefly, to make the cDNA library, one should choose a source that is rich in mRNA. The mRNA can then be made into cDNA, ligated into a recombinant vector, and transfected into a recombinant host for propagation, screening and cloning. For a genomic library, the DNA is extracted from a suitable tissue and either mechanically sheared or enzymatically digested to yield fragments of preferably about 5-100 kb. The fragments are then separated by gradient centrifugation from undesired sizes and are constructed in bacteriophage lambda vectors. These vectors and phage are packaged in vitro, and the recombinant phages are analyzed by plaque hybridization. Colony hybridization is carried out as generally described in Grunstein et al., Proc. Natl. Acad. Sci. USA., 72:3961-3965 (1975).

An alternative method combines the use of synthetic oligonucleotide primers with polymerase extension on an mRNA or DNA template. Suitable primers can be designed from specific sequences disclosed herein. This polymerase chain reaction (PCR) method amplifies the nucleic acids encoding the protein of interest directly from mRNA, cDNA, genomic libraries or cDNA libraries. Restriction endonuclease sites can be incorporated into the primers. Polymerase chain reaction or other in vitro amplification methods may also be useful, for example, to clone nucleic acids encoding specific proteins and express said proteins, to synthesize nucleic acids that will be used as probes for detecting the presence of mRNA encoding a polypeptide of the invention in physiological samples, for nucleic acid sequencing, or for other purposes (see, U.S. Pat. Nos. 4,683,195 and 4,683,202). Genes amplified by a PCR reaction can be purified from agarose gels and cloned into an appropriate vector.

Appropriate primers and probes for identifying the genes encoding a polypeptide of the invention from mammalian tissues can be derived from the sequences provided herein. For a general overview of PCR, see, Innis et al. PCR Protocols: A Guide to Methods and Applications, Academic Press, San Diego (1990).

Synthetic oligonucleotides can be used to construct genes. This is done using a series of overlapping oligonucleotides, usually 40-120 bp in length, representing both the sense and anti-sense strands of the gene. These DNA fragments are then annealed, ligated and cloned.

A polynucleotide encoding a polypeptide of the invention can be cloned using intermediate vectors before transformation into mammalian cells for expression. These intermediate vectors are typically prokaryote vectors or shuttle vectors. The proteins can be expressed in either prokaryotes or eukaryotes, using standard methods well known to those of skill in the art.

III. Purification of Proteins of the Invention

Either naturally occurring or recombinant polypeptides of the invention can be purified for use in functional assays. Naturally occurring polypeptides of the invention can be purified from any source (e.g., tissues of an organism expressing an ortholog). Recombinant polypeptides can be purified from any suitable expression system.

The polypeptides of the invention may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant polypeptides are being purified. For example, proteins having established molecular adhesion properties can be reversibly fused to a polypeptide of the invention. With the appropriate ligand, either protein can be selectively adsorbed to a purification column and then freed from the column in a relatively pure form. The fused protein may be then removed by enzymatic activity. Finally polypeptides can be purified using immunoaffinity columns.

A. Purification of Proteins from Recombinant Bacteria

When recombinant proteins are expressed by the transformed bacteria in large amounts, typically after promoter induction, although expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells typically, but not limited to, by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook et al., both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques.

Alternatively, it is possible to purify proteins from bacteria periplasm. Where the protein is exported into the periplasm of the bacteria, the periplasmic fraction of the bacteria can be isolated by cold osmotic shock in addition to other methods known to those of skill in the art (see, Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Purification of Proteins from Insect Cells

Proteins can also be purified from eukaryotic gene expression systems as described in, e.g., Fernandez and Hoeffier, Gene Expression Systems (1999). In some embodiments, baculovirus expression systems are used to isolate proteins of the invention. Recombinant baculoviruses are generally generated by replacing the polyhedrin coding sequence of a baculovirus with a gene to be expressed (e.g., encoding a polypeptide of the invention). Viruses lacking the polyhedrin gene have a unique plaque morphology making them easy to recognize. In some embodiments, a recombinant baculovirus is generated by first cloning a polynucleotide of interest into a transfer vector (e.g., a pUC based vector) such that the polynucleotide is operably linked to a polyhedrin promoter. The transfer vector is transfected with wildtype DNA into an insect cell (e.g., Sf9, Sf21 or BT1-TN-5B1-4 cells), resulting in homologous recombination and replacement of the polyhedrin gene in the wildtype viral DNA with the polynucleotide of interest. Virus can then be generated and plaque purified. Protein expression results upon viral infection of insect cells. Expressed proteins can be harvested from cell supernatant if secreted, or from cell lysates if intracellular. See, e.g., Ausubel et al. and Fernandez and Hoeffler, supra.

C. Standard Protein Separation Techniques for Purifying Proteins

1. Solubility Fractionation

Often as an initial step, and if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

2. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

3. Column Chromatography

The proteins of interest can also be separated from other proteins on the basis of their size, net surface charge, hydrophobicity and affinity for ligands. In addition, antibodies raised against proteins can be conjugated to column matrices and the proteins immunopurified. All of these methods are well known in the art.

Immunoaffinity chromatography using antibodies raised to a variety of affinity tags such as hemagglutinin (HA), FLAG, Xpress, Myc, hexahistidine (His), glutathione S transferase (GST) and the like can be used to purify polypeptides. The His tag will also act as a chelating agent for certain metals (e.g., Ni) and thus the metals can also be used to purify His-containing polypeptides. After purification, the tag is optionally removed by specific proteolytic cleavage.

It will be apparent to one of skill that chromatographic techniques can be performed at any scale and using equipment from many different manufacturers (e.g., Pharmacia Biotech).

IV. Detection of Polynucleotides of the Invention

Those of skill in the art will recognize that detection of expression of polynucleotides and polypeptides of the invention has many uses. For example, as discussed herein, detection of levels of polynucleotides and polypeptides of the invention in a patient is useful for diagnosing diabetes or a predisposition for at least some of the pathological effects of diabetes. Moreover, detection of gene expression is useful to identify modulators of expression of polynucleotides and polypeptides of the invention.

A variety of methods of specific DNA and RNA measurement that use nucleic acid hybridization techniques are known to those of skill in the art (see, Sambrook, supra). Some methods involve an electrophoretic separation (e.g., Southern blot for detecting DNA, and Northern blot for detecting RNA), but measurement of DNA and RNA can also be carried out in the absence of electrophoretic separation (e.g., by dot blot). Southern blot of genomic DNA (e.g., from a human) can be used for screening for restriction fragment length polymorphism (RFLP) to detect the presence of a genetic disorder affecting a polypeptide of the invention.

The selection of a nucleic acid hybridization format is not critical. A variety of nucleic acid hybridization formats are known to those skilled in the art. For example, common formats include sandwich assays and competition or displacement assays. Hybridization techniques are generally described in Hames and Higgins Nucleic Acid Hybridization, A Practical Approach, IRL Press (1985); Gall and Pardue, Proc. Natl. Acad. Sci. U.S.A., 63:378-383 (1969); and John et al. Nature, 223:582-587 (1969).

Detection of a hybridization complex may require the binding of a signal-generating complex to a duplex of target and probe polynucleotides or nucleic acids. Typically, such binding occurs through ligand and anti-ligand interactions as between a ligand-conjugated probe and an anti-ligand conjugated with a signal. The binding of the signal generation complex is also readily amenable to accelerations by exposure to ultrasonic energy.

The label may also allow indirect detection of the hybridization complex. For example, where the label is a hapten or antigen, the sample can be detected by using antibodies. In these systems, a signal is generated by attaching fluorescent or enzyme molecules to the antibodies or in some cases, by attachment to a radioactive label (see, e.g., Tijssen, “Practice and Theory of Enzyme Immunoassays,” Laboratory Techniques in Biochemistry and Molecular Biology, Burdon and van Knippenberg Eds., Elsevier (1985), pp. 9-20).

The probes are typically labeled either directly, as with isotopes, chromophores, lumiphores, chromogens, or indirectly, such as with biotin, to which a streptavidin complex may later bind. Thus, the detectable labels used in the assays of the present invention can be primary labels (where the label comprises an element that is detected directly or that produces a directly detectable element) or secondary labels (where the detected label binds to a primary label, e.g., as is common in immunological labeling). Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labeled probes or the like.

Other labels include, e.g., ligands that bind to labeled antibodies, fluorophores, chemiluminescent agents, enzymes, and antibodies that can serve as specific binding pair members for a labeled ligand. An introduction to labels, labeling procedures and detection of labels is found in Polak and Van Noorden Introduction to Immunocytochemistry, 2nd ed., Springer Verlag, N.Y. (1997); and in Haugland Handbook of Fluorescent Probes and Research Chemicals, a combined handbook and catalogue Published by Molecular Probes, Inc. (1996).

In general, a detector that monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill in the art. Commonly, an optical image of a substrate comprising bound labeling moieties is digitized for subsequent computer analysis.

The amount of, for example, an RNA is measured by quantitating the amount of label fixed to the solid support by binding of the detection reagent. Typically, the presence of a modulator during incubation will increase or decrease the amount of label fixed to the solid support relative to a control incubation that does not comprise the modulator, or as compared to a baseline established for a particular reaction type. Means of detecting and quantitating labels are well known to those of skill in the art.

In some embodiments, the target nucleic acid or the probe is immobilized on a solid support. Solid supports suitable for use in the assays of the invention are known to those of skill in the art. As used herein, a solid support is a matrix of material in a substantially fixed arrangement.

A variety of automated solid-phase assay techniques are also appropriate. For instance, very large scale immobilized polymer arrays (VLSIPS™), i.e. Gene Chips or microarrays, available from Affymetrix, Inc. in Santa Clara, Calif. can be used to detect changes in expression levels of a plurality of genes involved in the same regulatory pathways simultaneously. See, Tijssen, supra., Fodor et al. (1991) Science, 251: 767-777; Sheldon et al. (1993) Clinical Chemistry 39(4): 718-719, and Kozal et al. (1996) Nature Medicine 2(7): 753-759. Similarly, spotted cDNA arrays (arrays of cDNA sequences bound to nylon, glass or another solid support) can also be used to monitor expression of a plurality of genes.

Typically, the array elements are organized in an ordered fashion so that each element is present at a specified location on the substrate. Because the array elements are at specified locations on the substrate, the hybridization patterns and intensities (which together create a unique expression profile) can be interpreted in terms of expression levels of particular genes and can be correlated with a particular disease or condition or treatment. See, e.g., Schena et al., Science 270: 467-470 (1995)) and (Lockhart et al., Nature Biotech. 14: 1675-1680(1996)).

Hybridization specificity can be evaluated by comparing the hybridization of specificity-control polynucleotide sequences to specificity-control polynucleotide probes that are added to a sample in a known amount. The specificity-control target polynucleotides may have one or more sequence mismatches compared with the corresponding polynucleotide sequences. In this manner, whether only complementary target polynucleotides are hybridizing to the polynucleotide sequences or whether mismatched hybrid duplexes are forming is determined.

Hybridization reactions can be performed in absolute or differential hybridization formats. In the absolute hybridization for-mat, polynucleotide probes from one sample are hybridized to the sequences in a microarray format and signals detected after hybridization complex formation correlate to polynucleotide probe levels in a sample. In the differential hybridization format, the differential expression of a set of genes in two biological samples is analyzed. For differential hybridization, polynucleotide probes from both biological samples are prepared and labeled with different labeling moieties. A mixture of the two labeled polynucleotide probes is added to a microarray. The microarray is then examined under conditions in which the emissions from the two different labels are individually detectable. Sequences in the microarray that are hybridized to substantially equal numbers of polynucleotide probes derived from both biological samples give a distinct combined fluorescence (Shalon et al. PCT publication WO95/35505). In some embodiments, the labels are fluorescent labels with distinguishable emission spectra, such as Cy3 and Cy5 fluorophores.

After hybridization, the microarray is washed to remove nonhybridized nucleic acids and complex formation between the hybridizable array elements and the polynucleotide probes is detected. Methods for detecting complex formation are well known to those skilled in the art. In some embodiments, the polynucleotide probes are labeled with a fluorescent label and measurement of levels and patterns of fluorescence indicative of complex formation is accomplished by fluorescence microscopy, such as confocal fluorescence microscopy.

In a differential hybridization experiment, polynucleotide probes from two or more different biological samples are labeled with two or more different fluorescent labels with different emission wavelengths. Fluorescent signals are detected separately with different photomultipliers set to detect specific wavelengths. The relative abundances/expression levels of the polynucleotide probes in two or more samples are obtained.

Typically, microarray fluorescence intensities can be normalized to take into account variations in hybridization intensities when more than one microarray is used under similar test conditions. In some embodiments, individual polynucleotide probe/target complex hybridization intensities are normalized using the intensities derived from internal normalization controls contained on each microarray.

Detection of nucleic acids can also be accomplished, for example, by using a labeled detection moiety that binds specifically to duplex nucleic acids (e.g., an antibody that is specific for RNA-DNA duplexes). One example uses an antibody that recognizes DNA-RNA heteroduplexes in which the antibody is linked to an enzyme (typically by recombinant or covalent chemical bonding). The antibody is detected when the enzyme reacts with its substrate, producing a detectable product. Coutlee et al. (1989) Analytical Biochemistry 181:153-162; Bogulavski (1986) et al. J. Immunol. Methods 89:123-130; Prooijen-Knegt (1982) Exp. Cell Res. 141:397-407; Rudkin (1976) Nature 265:472-473, Stollar (1970) PNAS 65:993-1000; Ballard (1982) Mol. Immunol. 19:793-799; Pisetsky and Caster (1982) Mol. Immunol. 19:645-650; Viscidi et al. (1988) J. Clin. Microbial. 41:199-209; and Kiney et al. (1989) J. Clin. Microbiol. 27:6-12 describe antibodies to RNA duplexes, including homo and heteroduplexes. Kits comprising antibodies specific for DNA:RNA hybrids are available, e.g., from Digene Diagnostics, Inc. (Beltsville, Md.).

In addition to available antibodies, one of skill in the art can easily make antibodies specific for nucleic acid duplexes using existing techniques, or modify those antibodies that are commercially or publicly available. In addition to the art referenced above, general methods for producing polyclonal and monoclonal antibodies are known to those of skill in the art (see, e.g., Paul (ed) Fundamental Immunology, Third Edition Raven Press, Ltd., NY (1993); Coligan Current Protocols in Immunology Wiley/Greene, NY (1991); Harlow and Lane Antibodies: A Laboratory Manual Cold Spring Harbor Press, NY (1989); Stites et al. (eds.) Basic and Clinical Immunology (4th ed.) Lange Medical Publications, Los Altos, Calif., and references cited therein; Goding Monoclonal Antibodies: Principles and Practice (2d ed.) Academic Press, New York, N.Y., (1986); and Kohler and Milstein Nature 256: 495-497 (1975)). Other suitable techniques for antibody preparation include selection of libraries of recombinant antibodies in phage or similar vectors (see, Huse et al. Science 246:1275-1281 (1989); and Ward et al. Nature 341:544-546 (1989)). Specific monoclonal and polyclonal antibodies and antisera will usually bind with a K_{D} of at least about 0.1 μM, preferably at least about 0.01 μM or better, and most typically and preferably, 0.001 μM or better.

The nucleic acids used in this invention can be either positive or negative probes. Positive probes bind to their targets and the presence of duplex formation is evidence of the presence of the target. Negative probes fail to bind to the suspect target and the absence of duplex formation is evidence of the presence of the target. For example, the use of a wild type specific nucleic acid probe or PCR primers may serve as a negative probe in an assay sample where only the nucleotide sequence of interest is present.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including Taqman and molecular beacon probes can be used to monitor amplification reaction products, e.g., in real time.

An alternative means for determining the level of expression of the nucleic acids of the present invention is in situ hybridization. In situ hybridization assays are well known and are generally described in Angerer et al., Methods Enzymol. 152:649-660 (1987). In an in situ hybridization assay, cells, preferentially human cells from the cerebellum or the hippocampus, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

Single nucleotide polymorphism (SNP) analysis is also useful for detecting differences between alleles of the polynucleotides (e.g., genes) of the invention. SNPs linked to genes encoding polypeptides of the invention are useful, for instance, for diagnosis of diseases (e.g., diabetes) whose occurrence is linked to the gene sequences of the invention. For example, if an individual carries at least one SNP linked to a disease-associated allele of the gene sequences of the invention, the individual is likely predisposed for one or more of those diseases. If the individual is homozygous for a disease-linked SNP, the individual is particularly predisposed for occurrence of that disease (e.g., diabetes). In some embodiments, the SNP associated with the gene sequences of the invention is located within 300,000; 200,000; 100,000; 15,000; 50,000; or 10,000 base pairs from the gene sequence.

Various real-time PCR methods including, e.g., Taqman or molecular beacon-based assays (e.g., U.S. Pat. Nos. 5,210,015; 5,487,972; Tyagi et al., Nature Biotechnology 14:303 (1996); and PCT WO 95/13399 are useful to monitor for the presence of absence of a SNP. Additional SNP detection methods include, e.g., DNA sequencing, sequencing by hybridization, dot blotting, oligonucleotide array (DNA Chip) hybridization analysis, or are described in, e.g., U.S. Pat. No. 6,177,249; Landegren et al., Genome Research, 8:769-776 (1998); Botstein et al., Am J Human Genetics 32:314-331 (1980); Meyers et al., Methods in Enzymology 155:501-527 (1987); Keen et al., Trends in Genetics 7:5 (1991); Myers et al., Science 230:1242-1246 (1985); and Kwok et al., Genomics 23:138-144 (1994).

V. Immunological Detection of Polypeptides of the Invention

In addition to the detection of polynucloetides of the invention and gene expression using nucleic acid hybridization technology, one can also use immunoassays to detect polypeptides of the invention. Immunoassays can be used to qualitatively or quantitatively analyze polypeptides of the invention. A general overview of the applicable technology can be found in Harlow & Lane, Antibodies: A Laboratory Manual (1988).

A. Antibodies to Target Proteins or Other Immunogens

Methods for producing polyclonal and monoclonal antibodies that react specifically with a protein of interest or other immunogen are known to those of skill in the art (see, e.g., Coligan, supra; and Harlow and Lane, supra; Stites et al., supra and references cited therein; Goding, supra; and Kohler and Milstein Nature, 256:495-497 (1975)). Such techniques include antibody preparation by selection of antibodies from libraries of recombinant antibodies in phage or similar vectors (see, Huse et al., supra; and Ward et al., supra). For example, in order to produce antisera for use in an immunoassay, the protein of interest or an antigenic fragment thereof, is isolated as described herein. For example, a recombinant protein is produced in a transformed cell line. An inbred strain of mice or rabbits is immunized with the protein using a standard adjuvant, such as Freund's adjuvant, and a standard immunization protocol. Alternatively, a synthetic peptide derived from the sequences disclosed herein is conjugated to a carrier protein and used as an immunogen.

Polyclonal sera are collected and titered against the immunogen in an immunoassay, for example, a solid phase immunoassay with the immunogen immobilized on a solid support. Polyclonal antisera with a titer of 10⁴ or greater are selected and tested for their crossreactivity against proteins other than the polypeptides of the invention or even other homologous proteins from other organisms, using a competitive binding immunoassay. Specific monoclonal and polyclonal antibodies and antisera will usually bind with a K_{D} of at least about 0.1 mM, more usually at least about 1 μM, preferably at least about 0.1 μM or better, and most preferably, 0.01 μM or better.

A number of proteins of the invention comprising immunogens may be used to produce antibodies specifically or selectively reactive with the proteins of interest. Recombinant protein is the preferred immunogen for the production of monoclonal or polyclonal antibodies. Naturally occurring protein may also be used either in pure or impure form. Synthetic peptides made using the protein sequences described herein may also be used as an immunogen for the production of antibodies to the protein. Recombinant protein can be expressed in eukaryotic or prokaryotic cells and purified as generally described supra. The product is then injected into an animal capable of producing antibodies. Either monoclonal or polyclonal antibodies may be generated for subsequent use in immunoassays to measure the protein.

Methods of production of polyclonal antibodies are known to those of skill in the art. In brief, an immunogen, preferably a purified protein, is mixed with an adjuvant and animals are immunized. The animal's immune response to the immunogen preparation is monitored by taking test bleeds and determining the titer of reactivity to polypeptides of the invention. When appropriately high titers of antibody to the immunogen are obtained, blood is collected from the animal and antisera are prepared. Further fractionation of the antisera to enrich for antibodies reactive to the protein can be done if desired (see, Harlow and Lane, supra).

Monoclonal antibodies may be obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519 (1976)). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a binding fragment thereof by screening a DNA library from human B cells according to the general protocol outlined by Huse et al., supra.

Once target immunogen-specific antibodies are available, the immunogen can be measured by a variety of immunoassay methods with qualitative and quantitative results available to the clinician. For a review of immunological and immunoassay procedures in general see, Stites, supra. Moreover, the immunoassays of the present invention can be performed in any of several configurations, which are reviewed extensively in Maggio Enzyme Immunoassay, CRC Press, Boca Raton, Fla. (1980); Tijssen, supra; and Harlow and Lane, supra.

Immunoassays to measure target proteins in a human sample may use a polyclonal antiserum that was raised to full-length polypeptides of the invention or a fragment thereof. This antiserum is selected to have low cross-reactivity against other proteins and any such cross-reactivity is removed by immunoabsorption prior to use in the immunoassay.

B. Immunological Binding Assays

In some embodiments, a protein of interest is detected and/or quantified using any of a number of well-known immunological binding assays (see, e.g., U.S. Pat. Nos. 4,366,241; 4,376,110; 4,517,288; and 4,837,168). For a review of the general immunoassays, see also Asai Methods in Cell Biology Volume 37: Antibodies in Cell Biology, Academic Press, Inc. NY (1993); Stites, supra. Immunological binding assays (or immunoassays) typically utilize a “capture agent” to specifically bind to and often immobilize the analyte (e.g., full-length polypeptides of the present invention, or antigenic subsequences thereof). The capture agent is a moiety that specifically binds to the analyte. The antibody may be produced by any of a number of means well known to those of skill in the art and as described above.

Immunoassays also often utilize a labeling agent to bind specifically to and label the binding complex formed by the capture agent and the analyte. The labeling agent may itself be one of the moieties comprising the antibody/analyte complex. Alternatively, the labeling agent may be a third moiety, such as another antibody, that specifically binds to the antibody/protein complex.

In a preferred embodiment, the labeling agent is a second antibody bearing a label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al. J. Immunol., 111:1401-1406 (1973); and Akerstrom, et al. J. Immunol., 135:2589-2542 (1985)).

Throughout the assays, incubation and/or washing steps may be required after each combination of reagents. Incubation steps can vary from about 5 seconds to several hours, preferably from about 5 minutes to about 24 hours. The incubation time will depend upon the assay format, analyte, volume of solution, concentrations, and the like. Usually, the assays will be carried out at ambient temperature, although they can be conducted over a range of temperatures, such as 10° C. to 40° C.

1. Non-Competitive Assay Formats

Immunoassays for detecting proteins or analytes of interest from tissue samples may be either competitive or noncompetitive. Noncompetitive immunoassays are assays in which the amount of captured protein or analyte is directly measured. In one preferred “sandwich” assay, for example, the capture agent (e.g., antibodies specific for the polypeptides of the invention) can be bound directly to a solid substrate where it is immobilized. These immobilized antibodies then capture the polypeptide present in the test sample. The polypeptide of the invention thus immobilized is then bound by a labeling agent, such as a second labelled antibody specific for the polypeptide. Alternatively, the second antibody may lack a label, but it may, in turn, be bound by a labeled third antibody specific to antibodies of the species from which the second antibody is derived. The second can be modified with a detectable moiety, such as biotin, to which a third labeled molecule can specifically bind, such as enzyme-labeled streptavidin.

2. Competitive Assay Formats

In competitive assays, the amount of protein or analyte present in the sample is measured indirectly by measuring the amount of an added (exogenous) protein or analyte displaced (or competed away) from a specific capture agent (e.g., antibodies specific for a polypeptide of the invention) by the protein or analyte present in the sample. The amount of immunogen bound to the antibody is inversely proportional to the concentration of immunogen present in the sample. In a particularly preferred embodiment, the antibody is immobilized on a solid substrate. The amount of analyte may be detected by providing a labeled analyte molecule. It is understood that labels can include, e.g., radioactive labels as well as peptide or other tags that can be recognized by detection reagents such as antibodies.

Immunoassays in the competitive binding format can be used for cross-reactivity determinations. For example, the protein encoded by the sequences described herein can be immobilized on a solid support. Proteins are added to the assay and compete with the binding of the antisera to the immobilized antigen. The ability of the above proteins to compete with the binding of the antisera to the immobilized protein is compared to that of the protein encoded by any of the sequences described herein. The percent cross-reactivity for the above proteins is calculated, using standard calculations. Those antisera with less than 10% cross-reactivity with each of the proteins listed above are selected and pooled. The cross-reacting antibodies are optionally removed from the pooled antisera by immunoabsorption with the considered proteins, e.g., distantly related homologs.

The immunoabsorbed and pooled antisera are then used in a competitive binding immunoassay as described above to compare a second protein, thought to be perhaps a protein of the present invention, to the immunogen protein. In order to make this comparison, the two proteins are each assayed at a wide range of concentrations and the amount of each protein required to inhibit 50% of the binding of the antisera to the immobilized protein is determined. If the amount of the second protein required is less than 10 times the amount of the protein partially encoded by a sequence herein that is required, then the second protein is said to specifically bind to an antibody generated to an immunogen consisting of the target protein.

3. Other Assay Formats

In some embodiments, western blot (immunoblot) analysis is used to detect and quantify the presence of a polypeptide of the invention in the sample. The technique generally comprises separating sample proteins by gel electrophoresis on the basis of molecular weight, transferring the separated proteins to a suitable solid support (such as, e.g., a nitrocellulose filter, a nylon filter, or a derivatized nylon filter) and incubating the sample with the antibodies that specifically bind the protein of interest. For example, antibodies are selected that specifically bind to the polypeptides of the invention on the solid support. These antibodies may be directly labeled or alternatively may be subsequently detected using labeled antibodies (e.g., labeled sheep anti-mouse antibodies) that specifically bind to the antibodies against the protein of interest.

Other assay formats include liposome immunoassays (LIA), which use liposomes designed to bind specific molecules (e.g., antibodies) and release encapsulated reagents or markers. The released chemicals are then detected according to standard techniques (see, Monroe et al. (1986) Amer. Clin. Prod. Rev. 5:34-41).

4. Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most labels useful in such methods can be applied to the present invention. Thus, a label is any composition detectable by spectroscopic, photochemical, biochemical, immunochemical, electrical, optical or chemical means. Useful labels in the present invention include magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

The label may be coupled directly or indirectly to the desired component of the assay according to methods well known in the art. As indicated above, a wide variety of labels may be used, with the choice of label depending on the sensitivity required, the ease of conjugation with the compound, stability requirements, available instrumentation, and disposal provisions.

Non-radioactive labels are often attached by indirect means. The molecules can also be conjugated directly to signal generating compounds, e.g., by conjugation with an enzyme or fluorescent compound. A variety of enzymes and fluorescent compounds can be used with the methods of the present invention and are well-known to those of skill in the art (for a review of various labeling or signal producing systems which may be used, see, e.g., U.S. Pat. No. 4,391,904).

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple calorimetric labels may be detected directly by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need to be labeled and the presence of the target antibody is detected by simple visual inspection.

VI. Identification of Modulators of Polypeptides of the Invention

Modulators of a polypeptide of the invention, i.e. agonists or antagonists of a polypeptide's activity, or polypeptide's or polynucleotide's expression, are useful for treating a number of human diseases, including diabetes. For example, administration of modulators can be used to treat diabetic patients or prediabetic individuals to prevent progression, and therefore symptoms, associated with diabetes (including insulin resistance).

A. Agents that Modulate Polypeptides of the Invention

The agents tested as modulators of polypeptides of the invention can be any small chemical compound, or a biological entity, such as a protein, sugar, nucleic acid or lipid. Typically, test compounds will be small chemical molecules and peptides. Essentially any chemical compound can be used as a potential modulator or ligand in the assays of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). Modulators also include agents designed to reduce the level of mRNA encoding a polypeptide of the invention (e.g. antisense molecules, ribozymes, DNAzymes, small inhibitory RNAs and the like) or the level of translation from an mRNA (e.g., translation blockers such as an antisense molecules that are complementary to translation start or other sequences on an mRNA molecule). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In some embodiments, high throughput screening methods involve providing a combinatorial chemical or peptide library containing a large number of potential therapeutic compounds (potential modulator compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

B. Methods of Screening for Modulators of the Polypeptides of the Invention

A number of different screening protocols can be utilized to identify agents that modulate the level of expression or activity of a polynucleotide of a polypeptide of the invention in cells, particularly mammalian cells, and especially human cells. In general terms, the screening methods involve screening a plurality of agents to identify an agent that modulates the activity of a polypeptide of the invention by, e.g., binding to the polypeptide, preventing an inhibitor or activator from binding to the polypeptide, increasing association of an inhibitor or activator with the polypeptide, or activating or inhibiting expression of the polypeptide.

Any cell expressing a full-length polypeptide of the invention or a fragment thereof can be used to identify modulators. In some embodiments, the cells are eukaryotic cells lines (e.g., CHO or HEK293) transformed to express a heterologous polypeptide of the invention. In some embodiments, a cell expressing an endogenous polypeptide of the invention is used in screens. In other embodiments, modulators are screened for their ability to effect insulin responses.

I. Polypeptide Binding Assays

Preliminary screens can be conducted by screening for agents capable of binding to polypeptides of the invention, as at least some of the agents so identified are likely modulators of a polypeptide of the invention. Binding assays are also useful, e.g., for identifying endogenous proteins that interact with polypeptides of the invention. For example, antibodies, receptors or other molecules that bind polypeptides of the invention can be identified in binding assays.

Binding assays usually involve contacting a polypeptide of the invention with one or more test agents and allowing sufficient time for the protein and test agents to form a binding complex. Any binding complexes formed can be detected using any of a number of established analytical techniques. Protein binding assays include, but are not limited to, methods that measure co-precipitation or co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89. Other binding assays involve the use of mass spectrometry or NMR techniques to identify molecules bound to a polypeptide of the invention or displacement of labeled substrates. The polypeptides of the invention utilized in such assays can be naturally expressed, cloned or synthesized.

In addition, mammalian or yeast two-hybrid approaches (see, e.g., Bartel, P. L. et. al. Methods Enzymol, 254:241 (1995)) can be used to identify polypeptides or other molecules that interact or bind when expressed together in a host cell.

2. Polypeptide Activity

The activity of polypeptides of the invention can be assessed using a variety of in vitro and in vivo assays to determine functional, chemical, and physical effects, e.g., measuring ligand binding (e.g., radioactive or otherwise labeled ligand binding), second messengers (e.g., cAMP, cGMP, IP₃, DAG, or Ca²⁺), ion flux, phosphorylation levels, transcription levels, and the like. Furthermore, such assays can be used to test for inhibitors and activators of the polypeptides of the invention. Modulators can also be genetically altered versions of polypeptides of the invention.

The polypeptide of the assay will be selected from a polypeptide with substantial identity to a sequence disclosed herein, e.g., SEQ ID NO:2, SEQ ID NO:8, SEQ ID NO:12, SEQ ID NO:16, SEQ ID NO:22, SEQ ID NO:26, SEQ ID NO:28, or SEQ ID NO:32, or other conservatively modified variants thereof. Generally, the amino acid sequence identity will be at least 70%, optionally at least 85%, optionally at least 90-95% to the polypeptides exemplified herein. Optionally, the polypeptide of the assays will comprise a fragment of a polypeptide of the invention, such as an extracellular domain, transmembrane domain, cytoplasmic domain, ligand binding domain, subunit association domain, active site, and the like. Either a polypeptide of the invention or a domain thereof can be covalently linked to a heterologous protein to create a chimeric protein used in the assays described herein.

Modulators of polypeptide activity are tested using either recombinant or naturally occurring polypeptides of the invention. The protein can be isolated, expressed in a cell, expressed in a membrane derived from a cell, expressed in tissue or in an animal, either recombinant or naturally occurring. For example, tissue slices, dissociated cells, e.g., from tissues expressing polypeptides of the invention, transformed cells, or membranes can be used. Modulation is tested using one of the in vitro or in vivo assays described herein.

Modulator binding to polypeptides of the invention, a domain, or chimeric protein can be tested in solution, in a bilayer membrane, attached to a solid phase, in a lipid monolayer, or in vesicles. Binding of a modulator can be tested using, e.g., changes in spectroscopic characteristics (e.g., fluorescence, absorbance, refractive index), hydrodynamic (e.g., shape), chromatographic, or solubility properties.

Samples or assays that are treated with a potential moulator (e.g., a “test compound”) are compared to control samples without the test compound, to examine the extent of modulation. Control samples (untreated with activators or inhibitors) are assigned a relative activity value of 100. Inhibition of the polypeptides of the invention is achieved when the activity value relative to the control is about 90%, optionally 50%, optionally 25-0%. Activation of the polypeptides of the invention is achieved when the activity value relative to the control is 110%, optionally 150%, 200%, 300%, 400%, 500%, or 1000-2000%.

3. Expression Assays

Screening for a compound that modulates the expression of a polynucleotide or a polypeptide of the invention is also provided. Screening methods generally involve conducting cell-based assays in which test compounds are contacted with one or more cells expressing a polynucleotide or a polypeptide of the invention, and then detecting an increase or decrease in expression (either transcript or translation product). Assays can be performed with any cells that express a polynucleotide or a polypeptide of the invention.

Expression can be detected in a number of different ways. As described infra, the expression level of a polynucleotide of the invention in a cell can be determined by probing the mRNA expressed in a cell with a probe that specifically hybridizes with a transcript (or complementary nucleic acid derived therefrom) of a polynucleotide of the invention. Probing can be conducted by lysing the cells and conducting Northern blots or without lysing the cells using in situ-hybridization techniques. Alternatively, a polypeptide of the invention can be detected using immunological methods in which a cell lysate is probed with antibodies that specifically bind to the polypeptide.

The level of expression or activity of a polynucleotide or a polypeptide of the invention can be compared to a baseline value. The baseline value can be a value for a control sample or a statistical value that is representative of expression levels of a polynucleotide or a polypeptide of the invention for a control population (e.g., lean individuals as described herein) or cells (e.g., tissue culture cells not exposed to a modulator). Expression levels can also be determined for cells that do not express the polynucleotide or a polypeptide of the invention as a negative control. Such cells generally are otherwise substantially genetically the same as the test cells.

A variety of different types of cells can be utilized in the reporter assays. Cells that do not endogenously express a polypeptide of the invention can be prokaryotic, but are preferably eukaryotic. The eukaryotic cells can be any of the cells typically utilized in generating cells that harbor recombinant nucleic acid constructs. Exemplary eukaryotic cells include, but are not limited to, yeast, and various higher eukaryotic cells such as the HEK293, HepG2, COS, CHO and HeLa cell lines.

Various controls can be conducted to ensure that an observed activity is authentic including running parallel reactions with cells that lack the reporter construct or by not contacting a cell harboring the reporter construct with test compound. Compounds can also be further validated as described below.

4. Validation

Agents that are initially identified by any of the foregoing screening methods can be further tested to validate the apparent activity. Modulators that are selected for further study can be tested on the “classic” insulin responsive cell line, mouse 3T3-L1 adipocytes, muscle cells such as L6 cells and the like. Cells (e.g., adipocytes or muscle cells) are pre-incubated with the modulators and tested for acute (up to 4 hours) and chronic (overnight) effects on basal and insulin-stimulated GLUT4 translocation and glucose uptake.

Following such studies, validity of the modulators is tested in suitable animal models. The basic format of such methods involves administering a lead compound identified during an initial screen to an animal that serves as a model for humans and then determining if expression of activity of a polypeptide of the invention is in fact modulated.

The effect of the compound will be assessed in either diabetic animals or in diet induced insulin resistant animals. The blood glucose and insulin levels will be determined. The animal models utilized in validation studies generally are mammals of any kind. Specific examples of suitable animals include, but are not limited to, primates, mice and rats. For example, monogenic models of diabetes (e.g., ob/ob and db/db mice, Zucker rats and Zucker Diabetic Fatty rats etc) or polygenic models of diabetes (e.g., OLETF rats, GK rats, NSY mice, and KK mice) can be useful for validating modulation of a polypeptide of the invention in a diabetic or insulin resistant animal. In addition, transgenic animals expressing human polypeptides of the invention can be used to further validate drug candidates.

C. Solid Phase and Soluble High Throughput Assays

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 or more different compounds are possible using the integrated systems of the invention. In addition, microfluidic approaches to reagent manipulation can be used.

A molecule of interest (e.g., a polypeptide or polynucleotide of the invention, or a modulator thereof) can be bound to the solid-state component, directly or indirectly, via covalent or non-covalent linkage, e.g., via a tag. The tag can be any of a variety of components. In general, a molecule that binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, poly-His, etc.) Antibodies to molecules with natural binders such as biotin are also widely available and appropriate tag binders (see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody that recognizes the first antibody. In addition to antibody-antigen interactions, receptor-ligand interactions are also appropriate as tag and tag-binder pairs, such as agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I (1993)). Similarly, toxins and venoms, viral epitopes, hormones (e.g., opiates, steroids, etc.), intracellular receptors (e.g., which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-gly sequences of between about 5 and 200 amino acids. Such flexible linkers are known to those of skill in the art. For example, poly(ethelyne glycol) linkers are available from Shearwater Polymers, Inc., Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent that fixes a chemical group to the surface that is reactive with a portion of the tag binder. For example, groups that are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature (see, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank and Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

The invention provides in vitro assays for identifying, in a high throughput format, compounds that can modulate the expression or activity of a polypeptide of the invention. Control reactions that measure activity of a polypeptide of the invention in a cell in a reaction that does not include a potential modulator are optional, as the assays are highly uniform. Such optional control reactions are appropriate and increase the reliability of the assay. Accordingly, in some embodiments, the methods of the invention include such a control reaction. For each of the assay formats described, “no modulator” control reactions that do not include a modulator provide a background level of binding activity.

In some assays it will be desirable to have positive controls. At least two types of positive controls are appropriate. First, a known activator of a polypeptide or a polynucleotide of the invention can be incubated with one sample of the assay, and the resulting increase in signal resulting from an increased expression level or activity of a polypeptide or a polynucleotide of the invention are determined according to the methods herein. Second, a known inhibitor of a polypeptide or a polynucleotide of the invention can be added, and the resulting decrease in signal for the expression or activity of a polypeptide or a polynucleotide of the invention can be similarly detected. It will be appreciated that modulators can also be combined with activators or inhibitors to find modulators that inhibit the increase or decrease that is otherwise caused by the presence of the known modulator of a polypeptide or a polynucleotide of the invention.

VII. Compositions, Kits and Integrated Systems

The invention provides compositions, kits and integrated systems for practicing the assays described herein using nucleic acids or polypeptides of the invention, antibodies, etc.

The invention provides assay compositions for use in solid phase assays; such compositions can include, for example, one or more nucleic acids encoding a polypeptide of the invention immobilized on a solid support, and a labeling reagent. In each case, the assay compositions can also include additional reagents that are desirable for hybridization. Modulators of expression or activity of a polypeptide of the invention can also be included in the assay compositions.

The invention also provides kits for carrying out the assays of the invention. The kits typically include a probe that comprises an antibody that specifically binds to a polypeptide of the invention or a polynucleotide sequence encoding such polypeptides, and a label for detecting the presence of the probe. The kits may include at least one polynucleotide sequence encoding a polypeptide of the invention. Kits can include any of the compositions noted above, and optionally further include additional components such as instructions to practice a high-throughput method of assaying for an effect on expression of the genes encoding a polypeptide of the invention, or on activity of a polypeptide of the invention, one or more containers or compartments (e.g., to hold the probe, labels, or the like), a control modulator of the expression or activity of a polypeptide of the invention, a robotic armature for mixing kit components or the like.

The invention also provides integrated systems for high-throughput screening of potential modulators for an effect on the expression or activity of a polypeptide of the invention. The systems can include a robotic armature which transfers fluid from a source to a destination, a controller which controls the robotic armature, a label detector, a data storage unit which records label detection, and an assay component such as a microtiter dish comprising a well having a reaction mixture or a substrate comprising a fixed nucleic acid or immobilization moiety.

A number of robotic fluid transfer systems are available, or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to 96 well microtiter plates to set up several parallel simultaneous binding assays.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical image.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g., by fluorescent or dark field microscopic techniques.

VIII. Administration and Pharmaceutical Compositions

Modulators of the polypeptides of the invention (e.g., antagonists or agonists) can be administered directly to the mammalian subject for modulation of activity of a polypeptide of the invention in vivo. Administration is by any of the routes normally used for introducing a modulator compound into ultimate contact with the tissue to be treated and is well known to those of skill in the art. Although more than one route can be used to administer a particular composition, a particular route can often provide a more immediate and more effective reaction than another route.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17^{th} ed. 1985)).

The modulators (e.g., agonists or antagonists) of the expression or activity of the a polypeptide of the invention, alone or in combination with other suitable components, can be prepared for injection or for use in a pump device. Pump devices (also known as “insulin pumps”) are commonly used to administer insulin to patients and therefore can be easily adapted to include compositions of the present invention. Manufacturers of insulin pumps include Animas, Disetronic and MiniMed.

The modulators (e.g., agonists or antagonists) of the expression or activity of a polypeptide of the invention, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, or intrathecally. The formulations of compounds can be presented in unit-dose or multi-dose sealed containers, such as ampoules and vials. Solutions and suspensions can be prepared from sterile powders, granules, and tablets of the kind previously described. The modulators can also be administered as part of a prepared food or drug.

The dose administered to a patient, in the context of the present invention should be sufficient to induce a beneficial response in the subject over time. The optimal dose level for any patient will depend on a variety of factors including the efficacy of the specific modulator employed, the age, body weight, physical activity, and diet of the patient, on a possible combination with other drugs, and on the severity of the case of diabetes. It is recommended that the daily dosage of the modulator be determined for each individual patient by those skilled in the art in a similar way as for known insulin compositions. The size of the dose also will be determined by the existence, nature, and extent of any adverse side-effects that accompany the administration of a particular compound or vector in a particular subject.

In determining the effective amount of the modulator to be administered a physician may evaluate circulating plasma levels of the modulator, modulator toxicity, and the production of anti-modulator antibodies. In general, the dose equivalent of a modulator is from about 1 ng/kg to 10 mg/kg for a typical subject.

For administration, modulators of the present invention can be administered at a rate determined by the LD-50 of the modulator, and the side-effects of the modulator at various concentrations, as applied to the mass and overall health of the subject. Administration can be accomplished via single or divided doses.

The compounds of the present invention can also be used effectively in combination with one or more additional active agents depending on the desired target therapy (see, e.g., Turner, N. et al. Prog. Drug Res. (1998) 51: 33-94; Haffner, S. Diabetes Care (1998) 21: 160-178; and DeFronzo, R. et al. (eds.), Diabetes Reviews (1997) Vol. 5 No. 4). A number of studies have investigated the benefits of combination therapies with oral agents (see, e.g., Mahler, R., J. Clin. Endocrinol. Metab. (1999) 84: 1165-71; United Kingdom Prospective Diabetes Study Group: UKPDS 28, Diabetes Care (1998) 21: 87-92; Bardin, C. W.,(ed.), Current Therapy In Endocrinology And Metabolism, 6th Edition (Mosby—Year Book, Inc., St. Louis, Mo. 1997); Chiasson, J. et al., Ann. Intern. Med. (1994) 121: 928-935; Coniff, R. et al., Clin. Ther. (1997) 19: 16-26; Coniff, R. et al., Am. J. Med. (1995) 98: 443-451; and Iwamoto, Y. et al., Diabet. Med. (1996) 13 365-370; Kwiterovich, P. Am. J. Cardiol (1998) 82(12A): 3U-17U). These studies indicate that modulation of diabetes, among other diseases, can be further improved by the addition of a second agent to the therapeutic regimen. Combination therapy includes administration of a single pharmaceutical dosage formulation that contains a modulator of the invention and one or more additional active agents, as well as administration of a modulator and each active agent in its own separate pharmaceutical dosage formulation. For example, a modulator and a thiazolidinedione can be administered to the human subject together in a single oral dosage composition, such as a tablet or capsule, or each agent can be administered in separate oral dosage formulations. Where separate dosage formulations are used, a modulator and one or more additional active agents can be administered at essentially the same time (i.e., concurrently), or at separately staggered times (i.e., sequentially). Combination therapy is understood to include all these regimens.

One example of combination therapy can be seen in treating pre-diabetic individuals (e.g., to prevent progression into type 2 diabetes) or diabetic individuals (or treating diabetes and its related symptoms, complications, and disorders), wherein the modulators can be effectively used in combination with, for example, sulfonylureas (such as chlorpropamide, tolbutamide, acetohexamide, tolazamide, glyburide, gliclazide, glynase, glimepiride, and glipizide); biguanides (such as metformin); a PPAR beta delta agonist; a ligand or agonist of PPAR gamma such as thiazolidinediones (such as ciglitazone, pioglitazone (see, e.g., U.S. Pat. No. 6,218,409), troglitazone, and rosiglitazone (see, e.g., U.S. Pat. No. 5,859,037)); PPAR alpha agonists such as clofibrate, gemfibrozil, fenofibrate, ciprofibrate, and bezafibrate; dehydroepiandrosterone (also referred to as DHEA or its conjugated sulphate ester, DHEA-SO4); antiglucocorticoids; TNFα inhibitors; α-glucosidase inhibitors (such as acarbose, miglitol, and voglibose); amylin and amylin derivatives (such as pramlintide, (see, also, U.S. Pat. Nos. 5,902,726; 5,124,314; 5,175,145 and 6,143,718.)); insulin secretogogues (such as repaglinide, gliquidone, and nateglinide (see, also, U.S. Pat. Nos. 6,251,856; 6,251,865; 6,221,633; 6,174,856)), and insulin.

IX. Gene Therapy

Conventional viral and non-viral based gene transfer methods can be used to introduce nucleic acids encoding engineered polypeptides of the invention in mammalian cells or target tissues. Such methods can be used to administer nucleic acids encoding polypeptides of the invention to cells in vitro. In some embodiments, the nucleic acids encoding polypeptides of the invention are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds) (1995); and Yu et al., Gene Therapy 1:13-26 (1994).

Methods of non-viral delivery of nucleic acids encoding engineered polypeptides of the invention include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids encoding engineered polypeptides of the invention take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of polypeptides of the invention could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression of the polypeptides of the invention is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994)). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

pLASN and MFG-S are examples are retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1):10-20 (1997); Dranoffet al. Hum. Gene Ther. 1:111-2 (1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al. Gene Ther. 9:748-55 (1996)).

Replication-deficient recombinant adenoviral vectors (Ad) can be engineered such that a transgene replaces the Ad E1a, E1b, and E3 genes; subsequently the replication defector vector is propagated in human 293 cells that supply deleted gene function in trans. Ad vectors can transduce multiply types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., PNAS 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In some embodiments, cells are isolated from the subject organism, transfected with a nucleic acid (gene or cDNA) encoding a polypeptides of the invention, and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al. J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and Iad (differentiated antigen presenting cells) (see Inaba et al., J. Exp. Med. 176:1693-1702 (1992)).

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention, as described below (see, e.g., Remington's Pharmaceutical Sciences, 17th ed., 1989).

X. Diagnosis of Diabetes

The present invention also provides methods of diagnosing diabetes or a predisposition of at least some of the pathologies of diabetes. Diagnosis can involve determination of a genotype of an individual (e.g., with SNPs) and comparison of the genotype with alleles known to have an association with the occurrence of diabetes. Alternatively, diagnosis also involves determining the level of a polypeptide or polynucleotide of the invention in a patient and then comparing the level to a baseline or range. Typically, the baseline value is representative of a polypeptide or polynucleotide of the invention in a healthy (e.g., lean) person.

As discussed above, variation of levels (e.g., low or high levels) of a polypeptide or polynucleotide of the invention compared to the baseline range indicates that the patient is either diabetic or at risk of developing at least some of the pathologies of diabetes (e.g., pre-diabetic). The level of a polypeptide in a lean individual can be a reading from a single individual, but is typically a statistically relevant average from a group of lean individuals. The level of a polypeptide in a lean individual can be represented by a value, for example in a computer program.

In some embodiments, the level of polypeptide or polynucleotide of the invention is measured by taking a blood, urine or tissue sample from a patient and measuring the amount of a polypeptide or polynucleotide of the invention in the sample using any number of detection methods, such as those discussed herein. For instance, fasting and fed blood or urine levels can be tested.

In some embodiments, the baseline level and the level in a lean sample from an individual, or at least two samples from the same individual differ by at least about 5%, 10%, 20%, 50%, 75%, 100%, 150%, 200%, 300%, 400%, 500%, 1000% or more. In some-embodiments, the sample from the individual is greater by at least one of the above-listed percentages relative to the baseline level. In some embodiments, the sample from the individual is lower by at least one of the above-listed percentages relative to the baseline level.

In some embodiments, the level of a polypeptide or polynucleotide of the invention is used to monitor the effectiveness of antidiabetic therapies such as thiazolidinediones, metformin, sulfonylureas and other standard therapies. In some embodiments the activity or expression of a polypeptide or polynucleotide of the invention will be measured prior to and after treatment of diabetic or pre-diabetic patients with antidiabetic therapies as a surrogate marker of clinical effectiveness. For example, the greater the reduction in expression or activity of a polypeptide of the invention indicates greater effectiveness.

Glucose/insulin tolerance tests can also be used to detect the effect of glucose levels on levels of a polypeptide or polynucleotide of the invention. In glucose tolerance tests, the patient's ability to tolerate a standard oral glucose load is evaluated by assessing serum and urine specimens for glucose levels. Blood samples are taken before the glucose is ingested, glucose is given by mouth, and blood or urine glucose levels are tested at set intervals after glucose ingestion. Similarly, meal tolerance tests can also be used to detect the effect of insulin or food, respectively, on levels of a polypeptide or polynucleotide of the invention.

All publications, accession numbers, and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

The following examples are offered to illustrate, but not to limit the claimed invention.

In obese individuals, peripheral tissues especially muscle and fat are known to have a moderately impaired ability to respond to insulin and hence to take up glucose. This defect in glucose metabolism is usually compensated for by increased secretion of insulin from the pancreas, thereby maintaining normal glucose levels. The majority of glucose disposal occurs in the muscle. A number of obese insulin resistant patients will progress to overt diabetics over time. The molecular defects underlying this peripheral insulin resistance in obese individuals are not well defined. Genes in muscle or fat whose expression is altered in obese individuals when compared to lean individuals may be causative genes for insulin resistance and also may predict the transition to diabetes. Modulators of such genes have the ability to reverse insulin resistance and restore insulin sensitivity to normal thereby improving whole body glucose homeostasis including for example insulin secretion.

Modulators of such genes also have the ability to be used to pre-empt the transition from obesity-induced insulin resistance to diabetes. For these reasons, gene expression profiling was performed in muscle from lean and obese individuals.

Gene Expression Profiling

Gene expression profiling was performed on tissue samples (muscle) obtained from lean obese and diabetic individuals. Two studies were performed. In the first study, basal samples were isolated from all individuals at the beginning of a 5 hour hyperinsulinemic euglycemic clamp. Clamp samples were isolated at the end of this procedure. Similar basal and clamp samples were taken 3 months later after all patients had taken the insulin sensitizing drug troglitazone (tro).

In the second study, samples were obtained from lean, obese, and diabetic individuals before and after a hyperinsulinemic euglycemic clamp. No troglitazone treatment was used. For all tissue samples mRNA was isolated from these muscle samples and converted to cRNA by standard procedures. The gene expression profile for each individual was determined by hybridization of cRNA to custom synthesized Affymetrix chips.

Gene expression profile differences were calculated as follows. The expression level of a particular gene is indicated by its ‘average difference score’. The raw data was analyzed by a statistical test to remove ‘outliers’. The mean ‘average difference score’ was then calculated from the average difference scores for all individuals in a particular treatment group. Genes were determined to be changed in condition 1 (such as basal leans) versus condition 2 (such as basal obese) by calculating the Students t test statistic between the two conditions and selecting those with t less than or equal to 0.05. Fold change was determined as the ratio of mean average difference score in condition 2 to the mean average difference score in condition 1. Genes that exhibit modulated expression in lean vs. obese patients are shown in Table 1.

Probe set MBXHUMMUS15644 detects P2RY1 nucleic acid sequences. Expression of transcripts encoding P2RY1 was increased in obese compared to lean individuals in this study.

Probe set MBXHUMMUS17820 detects PTPLA nucleic acid sequences. Expression of PTPLA transcripts was decreased in obese compared to lean individuals in this study.

Probe set MBXHUMMUS18195 detects CRIM1 nucleic acid sequences. Expression of CRM1 transcripts was increased in obese compared to lean individuals in this study.

Probe set MBXHUMMUS28977 detects PPP3CA nucleic acid sequences. Expression of transcripts encoding PPP3CA was increased in obese compared to lean individuals in this study.

Probe set MBXHUMMUS29021 detects PTPN3 nucleic acid sequences. Expression of PTPN3 transcripts was decreased in obese compared to lean individuals in this study.

Probe set MBXHUMMUS30183 detects a transcript encoding DUSP3. Expression of transcripts encoding DUSP3 was decreased in obese compared to lean individuals in this study.

Probe set MBXHUMMUS32423 detects RGS10 nucleic acid sequences. Expression of transcripts encoding RGS10 was increased in obese compared to lean individuals in this study.

TABLE 1
Genes exhibiting modulated expression in lean vs obese individuals

| | Lean Pre-Trog | | | Obese Pre-Trog | | | Fold | Students | |
|---|---|---|---|---|---|---|---|---|---|
| B/C | Mean Expr | SEM | n | Mean Expr | SEM | n | Change | t test | Gene name |
| B | 91 | 21 | 7 | 290 | 18 | 7 | 3.21 | 0.000 | P2RY1 |
| B | 822 | 81 | 7 | 488 | 28 | 7 | 0.59 | 0.005 | PTPLA |
| B | 757 | 122 | 7 | 1804 | 153 | 7 | 2.38 | 0.001 | CRIM1 |
| B | 399 | 53 | 7 | 849 | 114 | 7 | 2.13 | 0.006 | PPP3CA |
| B | 2403 | 226 | 7 | 1429 | 64 | 7 | 0.59 | 0.004 | PTPN3 |
| B | 3578 | 208 | 7 | 2409 | 302 | 7 | 0.67 | 0.010 | DUSP3 |
| B | 181 | 12 | 17 | 273 | 38 | 16 | 1.51 | 0.031 | RGS10 |

Legend: B/C indicates sample is from Basal or Clamp; “Pre-Trog” indicates sample was taken before 3 months of Troglitazone treatment; “Mean Expr” indicates mean expression; “SEM” indicates standard error of mean; “n” indicates number of patient samples; “Fold Change” indicates fold change of obese in comparison to lean patients.

Real-Time Quantitative PCR and Modulation of Insulin Sensitivity

Modulated expression of the genes identified in the profiling studies was further confirmed by real-time PCR. The ability of the genes to modulate insulin sensitivity was also assessed.

Methodology

The gene expression changes observed in the human muscle samples derived from lean and obese individuals were further confirmed by the use of real-time quantitative PCR. A combination of specific PCR primers and a Taqman probe were designed to detect and quantify expression levels for each gene. Relative gene expression levels were determined in this manner in each patient and the mean expression level in either the lean or obese patient population was then calculated.

In order to demonstrate the ability of the gene to modulate insulin sensitivity a number of different analyses were undertaken. One analysis called “Glut 4 translocation” involves over-expressing the gene in 3T3-L1 adipocytes and then monitoring the ability of insulin to cause the movement (translocation) of the major insulin-regulated glucose transporter, Glut 4, to the cell surface. The cDNA encoding the human gene was sub-cloned into the mammalian expression vector pcDNA3.1 which provides for a V5 epitope tag. This plasmid encoding the human gene was then introduced into differentiated murine 3T3-L1 adipocytes along with a cloned Glut 4 expression construct using an electroporation procedure essentially as described in Kanzaki et al., J. Biol. Chem 275:7167-7175 (2000). A plasmid expressing LacZ was used as a negative control. After stimulation with increasing concentrations of insulin, cells co-expressing both the human gene and cloned Glut 4 were scored for the presence of Glut 4 at the cell surface using fluorescence microscopy.

In some instances another analysis called “glucose transport” was used. This involves over-expressing the gene in either differentiated 3T3-L1 adipocytes or differentiated L6 myotubes and then monitoring the ability of insulin to stimulate glucose transport into these two cell types. A recombinant adenovirus encoding the human gene tagged with a FLAG epitope tag was prepared essentially as described in Zhou et al., Proc. Nat. Acad. Sci. USA. 95:2509-2514 (1998). 3T3-L1 adipocytes or L6 myotubes were infected with recombinant adenovirus expressing the human gene. A virus expressing an irrelevant protein was used as the control. Twenty four hours post infection the cells are stimulated with increasing concentrations of insulin and glucose transport into the cells determined by the counting the accumulation of a radiolabelled glucose analog (2-deoxyglucose) essentially as described in Kotani et al., Mol. Cell. Biol. 18:6971-6982 (1998), Fujishiro et al., J. Biol. Chem. 276:19800-19806 (2001), Ross et al., Biochem. Biophys. Res. Commun. 302:354-358 (2003)

Results

The results of the real-time PCR and insulin sensitivity analyses are provided below for the genes shown in Table 1.

P2RY1

The results of the real-time PCR analysis further show that P2RY1 is significantly over-expressed in muscle from obese individuals when compared to muscle from lean individuals.

| Comparison | Expression Fold change | t test |
|---|---|---|
| Obese (7)/Lean (7) | 1.98 | 0.001 |

Legend “Fold change” indicates fold change in P2RY1 expression calculated as the ratio of mean obese expression/mean lean expression. Numbers in parentheses indicate the number of patient samples analyzed by real time PCR.

Glucose transport analysis in 3T3-L1 adipocytes:

| | Fold Change 1 | | Fold Change 2 | |
|---|---|---|---|---|
| | (Basal plus 2-MeSADP)/ | | (Basal plus P2RY1 plus 2-MeSADP)/ | |
| Expt. | (Basal no 2-MeSADP) | t test 1 | (Basal no P2RY1 no 2-MeSADP) | t test 2 |
| 1 | 1.52 | 0.091 | 3.38 | 0.006 |
| 2 | 0.69 | 0.075 | 1.38 | 0.03 |
| 3 | 1.11 | 0.715 | 2.11 | 0.154 |
| 4 | 0.94 | 0.788 | 1.84 | 0.095 |
| 5 | 1.05 | 0.782 | 1.86 | 0.087 |
| Mean +/− | 1.07 +/− 0.13 | | 2.11 +/− 0.34 | N/A |
| SEM | | | | |
| (n = 5) | | | | |

Legend Fold Change is defined as the following ratio; glucose transport in hP2RY1-expressing cells/glucose transport in non-hP2RY1-expressing cells. SEM is the standard error of the mean. Basal is defined as cells un-stimulated by insulin. Cells were treated with a P2RY1 agonist, 2-MeSADP, at a final concentration of 1 μM for 6 hours prior to insulin stimulation, “h” is human.

The fold stimulation of basal glucose transport in the presence of hP2RY1 and 2-MeSADDP compared to the fold stimulation of basal glucose transport in the presence of 2-MeSADDP alone was also found to be highly significant. For this comparison the the t test was calculated to be 0.02.

These data show that basal glucose transport in 3T3-L1 adipocytes can be stimulated by a P2RY1 agonist but only when the hP2RY1 is expressed in the cells. The murine P2RY1 does not appear to be expressed in 3T3-L1 adipocytes since it could not be detected either at the RNA or protein levels. These finding explains the lack of stimulation of basal glucose transport by the P2RY1 agonist alone in 3T3-L1 adipocytes.

Glucose transport analysis in L6 myotubes:

| | Fold Change 1 | | Fold Change 2 | |
|---|---|---|---|---|
| | (Basal plus 2-MeSADP)/ | | (Basal plus P2RY1 plus 2-MeSADP)/ | |
| Expt. | (Basal no 2-MeSADP) | t test 1 | (Basal no P2RY1 no 2-MeSADP) | t test 2 |
| 1 | 0.99 | 0.768 | 1.71 | 3.38E−06 |
| 2 | 0.95 | 0.390 | 1.91 | 3.39E−03 |
| Mean | 0.97 | | 1.81 | N/A |
| (n = 2) | | | | |

Legend Fold Change is defined as the following ratio; glucose transport in hP2RY1-expressing cells/glucose transport in non-hP2RY1-expressing cells. SEM is the standard error of the mean. Basal is defined as cells un-stimulated by insulin. Cells were treated with a P2RY1 agonist, 2-MeSADP, at a final concentration of 1 μM for 6 hours prior to insulin stimulation.

The fold stimulation of basal glucose transport in the presence of hP2RY1 and 2-MeSADP compared to the fold stimulation of basal glucose transport in the presence of 2-MeSADP alone was also found to be highly significant. For this comparison the the t test was calculated to be 0.0136.

These data show that basal glucose transport in L6 myotubes can be stimulated by a P2RY1 agonist but only when the hP2RY1 is expressed in the cells. This further confirms the conclusion that basal glucose transport can be stimulated in cells such as adipocytes or myotubes by a P2RY1 agonist provided these cells express sufficient P2RY1 levels.

PTPLA

The results of the real-time PCR analysis further show that PTPLA expression is significantly reduced in muscle from obese individuals when compared to muscle from lean individuals.

| Comparison | Expression Fold change | t test |
|---|---|---|
| Obese (7)/Lean (7) | 0.59 | 0.002 |

Legend “Fold change” indicates fold change in PTPLA expression calculated as the ratio of mean obese expression/mean lean expression. Numbers in parentheses indicate the number of patient samples analyzed by real time PCR.

Glut 4 translocation analysis:

| Insulin | Fold Change (Mean hPTPLA/ | t test |
|---|---|---|
| (nM) | Mean LacZ) (n = 3) | (hPTPLA vs LacZ) |
| 0 | 15.11 | 1.4E−04 |
| 1 | 1.32 | 0.028 |
| 10 | 1.00 | 0.991 |

Legend “Fold Change” indicates the following ratio; (Mean % of PTPLA-expressing cells scored positive for cell surface Glut 4)/(Mean % of LacZ-expressing cells scored positive for cell surface Glut 4). “h” is human. “n” is the number of replicates.

These data show that increasing the levels of hPTPLA in 3T3-L1 adipocytes significantly stimulates basal and sub-maximal glucose transport with little effect on maximal glucose transport.

CRIM1

The results of the real-time PCR analysis further show that CRM1 is significantly over-expressed in muscle from obese individuals when compared to muscle from lean individuals.

| Comparison | Expression Fold change | t test |
|---|---|---|
| Obese (7)/Lean (7) | 2.21 | 0.014 |

Legend “Fold change” indicates fold change in CRIM1 expression calculated as the ratio of mean obese expression/mean lean expression. Numbers in parentheses indicate the number of patient samples analyzed by real time PCR.

Glut 4 translocation analysis:

| Insulin | Fold Change (Mean hCRIM1/ | t test |
|---|---|---|
| (nM) | Mean LacZ) (n = 3) | (hCRIM1 vs LacZ) |
| 0 | 0.32 | 0.033 |
| 1 | 0.72 | 0.627 |
| 10 | 0.69 | 0.372 |

Legend “Fold Change” indicates the following ratio; (Mean % of CRIM1-expressing cells scored positive for cell surface Glut 4)/(Mean % of LacZ-expressing cells scored positive for cell surface Glut 4). “h” is human, “n” is the number of replicates.

These data show that increasing the levels of hCRIM1 in 3T3-L1 adipocytes significantly inhibits basal glucose transport with little effect on maximal glucose transport.

PPP3CA

Real time PCR further shows that PPP3CA is significantly over-expressed in muscle from obese individuals when compared to muscle from lean individuals.

| Comparison | Expression Fold change | t test |
|---|---|---|
| Obese (7)/Lean (7) | 1.59 | 0.081 |

Legend “Fold change” indicates fold change in PPP3CA expression calculated as the ratio of mean obese expression/mean lean expression. Numbers in parentheses indicate the number of patient samples analyzed by real time PCR.

Glut 4 translocation analysis:

| Insulin | Fold Change (Mean hPPP3CA/ | t test |
|---|---|---|
| (nM) | Mean LacZ) (n = 6) | (hPPP3CA vs LacZ) |
| 0 | 5.87 | 0.001 |
| 1 | 0.63 | 0.036 |
| 10 | 0.75 | 0.002 |

Legend “Fold Change” indicates the following ratio; (Mean % of hPPP3CA-expressing cells scored positive for cell surface Glut 4)/(Mean % of LacZ-expressing cells scored positive for cell surface Glut 4). “h” is human. “n” is the number of replicates.

These data show that increasing the levels of hPPP3CA in 3T3-L1 adipocytes significantly simulates basal Glut 4 translocation while also significantly inhibiting submax and maximum insulin stimulated Glut 4 translocation.

Glucose transport analysis in 3T3-L1 adipocytes:

| | Expt 1 | | Expt 2 | | Expt 3 | | Mean FC |
|---|---|---|---|---|---|---|---|
| | FC | | FC | | FC | | (hPPP3CA/ |
| Insulin | (hPPP3CA/ | | (hPPP3CA/ | | (hPPP3CA/ | | Con) +/− SEM |
| (nM) | Con) | t test | Con) | t test | Con) | t test | (n = 3) |
| 0 | 0.8 | 0.078 | 1.0 | 0.873 | 1.6 | 0.069 | 1.13 +/− 0.25 |
| 0.05 | 0.9 | 0.631 | 1.1 | 0.134 | 1.3 | 0.287 | 1.12 +/− 0.10 |
| 0.1 | 1.3 | 0.414 | 1.1 | 0.260 | 1.0 | 0.774 | 1.13 +/− 0.07 |
| 0.3 | 1.7 | 0.111 | 1.0 | 0.676 | 1.0 | 0.921 | 1.23 +/− 0.21 |
| 1 | 1.8 | 0.066 | 1.0 | 0.317 | 0.9 | 0.319 | 1.26 +/− 0.29 |
| 10 | 1.2 | 0.151 | 0.8 | 0.035 | 0.9 | 0.293 | 0.98 +/− 0.11 |

Legend “Con” indicates control cells which do not express PPP3CA. “FC” indicates the fold change defined as the following ratio; glucose transport in PPP3CA-expressing cells/glucose transport in non-PPP3CA-expressing cells. “h” is human. “n” is the number of replicates. SEM is the standard error of the mean.

These data show that increasing the levels of hPPP3CA in 3T3-L1 adipocytes has no significant effect on basal or insulin stimulated glucose transport.

Glucose transport analysis in L6 myotubes:

| | Expt 1 | | Expt 2 | | Expt 3 | | Mean FC |
|---|---|---|---|---|---|---|---|
| | FC | | FC | | FC | | (hPPP3CA/ |
| | (hPPP3CA/ | | (hPPP3CA/ | | (hPPP3CA/ | | Con) +/− SEM |
| Insulin | Con) | t test | Con) | t test | Con) | t test | (n = 3) |
| 0 | 1.03 | 0.480 | 0.93 | 0.238 | 0.91 | 0.360 | 0.96 +/− 0.04 |
| 10 | 0.77 | 0.003 | 0.93 | 0.400 | 0.82 | 0.271 | 0.84 +/− 0.05 |
| 100 | 0.85 | 0.013 | 0.94 | 0.350 | 1.08 | 0.489 | 0.96 +/− 0.07 |

Legend “Con” indicates control cells which do not express PPP3CA. “FC” indicates the fold change defined as the following ratio; glucose transport in PPP3CA-expressing cells/glucose transport in non-PPP3CA-expressing cells. “h” is human. “n” is the number of replicates. SEM is the standard error of the mean.

These data show that increasing the levels of hPPP3CA in L6 myotubes has no significant effects on basal or insulin stimulated glucose transport.

PTPN3

Real time PCR analysis shows that PTPN3 has significantly reduced expression in muscle from obese individuals when compared to muscle from lean individuals.

| Comparison | Expression Fold change | t test |
|---|---|---|
| Obese (7)/Lean (7) | 0.56 | 0.061 |

Legend “Fold change” indicates fold change in PTPN3 expression calculated as the ratio of mean obese expression/mean lean expression. Numbers in parentheses indicate the number of patient samples analyzed by real time PCR.

Glut 4 translocation analysis:

| Insulin | Fold Change (Mean hPTPN3a/ | t test |
|---|---|---|
| (nM) | Mean LacZ) (n = 3) | (hPTPN3a vs LacZ) |
| 0 | 1.78 | 0.217 |
| 1 | 0.42 | 0.012 |
| 10 | 0.36 | 0.004 |

Legend “Fold Change” indicates the following ratio; (Mean % of hPTPN3a-expressing cells scored positive for cell surface Glut 4)/(Mean % of LacZ-expressing cells scored positive for cell surface Glut 4). h” is human. “n” is the number of replicates.

These data show that increasing the levels of hPTPN3a in 3T3-L1 adipocytes significantly inhibited Glut 4 translocation at all insulin concentrations tested with little or no effect on basal Glut 4 translocation.

| Insulin | Fold Change (Mean hPTPN3b/ | t test |
|---|---|---|
| (nM) | Mean LacZ) (n = 3) | (hPTPN3b vs LacZ) |
| 0 | 1.76 | 0.241 |
| 1 | 0.30 | 0.005 |
| 10 | 0.29 | 0.001 |

Legend “Fold Change” indicates the following ratio; (Mean % of PTPN3b-expressing cells scored positive for cell surface Glut 4)/(Mean % of LacZ-expressing cells scored positive for cell surface Glut 4). h” is human. “n” is the number of replicates.

These data show that increasing the levels of hPTPN3b, a novel variant of PTPN3a, in 3T3-L1 adipocytes significantly inhibited Glut 4 translocation at all insulin concentrations tested with little or no effect on basal Glut 4 translocation.

DUSP3

Real time PCR analysis shows that DUSP3 has significantly reduced expression in muscle from obese individuals when compared to muscle from lean individuals.

| Comparison | Expression Fold change | t test |
|---|---|---|
| Obese (7)/Lean (7) | 0.66 | 0.110 |

Legend “Fold change” indicates fold change in DUSP3 expression calculated as the ratio of mean obese expression/mean lean expression. Numbers in parentheses indicate the number of patient samples analyzed by real time PCR.

Glut 4 translocation analysis:

| Insulin | Fold Change (Mean hDUSP3/ | t test |
|---|---|---|
| (nM) | Mean LacZ) (n = 3) | (hDUSP3 vs LacZ) |
| 0 | 1.4 | 0.156 |
| 0.5 | 2.1 | 0.002 |
| 10 | 1.1 | 0.612 |

Legend “Fold Change” indicates the following ratio; (Mean % of DUSP3-expressing cells scored positive for cell surface Glut 4)/(Mean % of LacZ-expressing cells scored positive for cell surface Glut 4). h” is human. “n” is the number of replicates.

These data show that increasing the levels of hDUSP3 in 3T3-L1 adipocytes improves Glut 4 translocation at sub-maximal (0.5 nM) with little or no significant effect on basal or maximal Glut 4 translocation. This improvement in sub-maximal Glut 4 translocation can be viewed as an increase in insulin sensitivity. Increasing DUSP3 therefore increased insulin sensitivity in these cells.

Glucose transport analysis in 3T3-L1 adipocytes:

| | Expt 1 | | Expt 2 | | Expt 3 | | |
|---|---|---|---|---|---|---|---|
| | FC | | FC | | FC | | Mean FC |
| Insulin | (hDUSP3/ | | (hDUSP3/ | | (hDUSP3/ | | (hDUSP3/Con) +/− SEM |
| (nM) | Con) | t test | Con) | t test | Con) | t test | (n = 3) |
| 0 | 1.1 | 0.406 | 1.3 | 0.114 | 1.0 | 0.757 | 1.12 +/− 0.086 |
| 0.05 | 1.3 | 0.346 | 1.3 | 0.288 | 1.0 | 0.953 | 1.18 +/− 0.097 |
| 0.1 | 1.2 | 0.399 | 1.3 | 0.075 | 1.2 | 0.287 | 1.23 +/− 0.041 |
| 0.3 | 1.1 | 0.616 | 1.3 | 0.185 | 1.0 | 0.900 | 1.12 +/− 0.077 |
| 1 | 1.0 | 0.748 | 1.2 | 0.124 | 0.9 | 0.213 | 1.04 +/− 0.084 |
| 10 | 1.0 | 0.866 | 1.1 | 0.438 | 0.8 | 0.002 | 0.96 +/− 0.074 |

Legend “Con” indicates control cells which do not express DUSP3. “FC” indicates the fold change defined as the following ratio; glucose transport in DUSP3-expressing cells/glucose transport in non-DUSP3-expressing cells. h” is human. “n” is the number of replicates. SEM is the standard error of the mean.

These data show that increasing the levels of hDUSP3 in 3T3-L1 adipocytes has no significant effects on basal or insulin stimulated glucose transport.

Glucose transport analysis in L6 myotubes

| | Expt 1 | | Expt 2 | | Expt 3 | | |
|---|---|---|---|---|---|---|---|
| | FC | | FC | | FC | | Mean FC |
| Insulin | (hDUSP3/ | | (hDUSP3/ | | (hDUSP3/ | | (hDUSP3/Con) +/− SEM |
| (nM) | Con) | t test | Con) | t test | Con) | t test | (n = 3) |
| 0 | 0.84 | 0.008 | 0.77 | 0.001 | 0.93 | 0.027 | 0.85 +/− 0.05 |
| 10 | 0.88 | 0.001 | 0.82 | 0.001 | 0.89 | 0.022 | 0.86 +/− 0.02 |
| 100 | 0.90 | 0.002 | 0.92 | 0.021 | 0.97 | 0.074 | 0.93 +/− 0.02 |

Legend “Con” indicates control cells which do not express hDUSP3. “FC” indicates the fold change defined as the following ratio; glucose transport in hDUSP3-expressing cells/glucose transport in non-hDUSP3-expressing cells. h” is human. “n” is the number of replicates. SEM is the standard error of the mean.

These data show that increasing the levels of hDUSP3 in L6 myotubes has a significant inhibitory effect on both basal and insulin stimulated glucose transport.

RGS10

Real time PCR analysis shows that RGS10 is significantly over-expressed in muscle from obese individuals when compared to muscle from lean individuals.

| Comparison | Expression Fold change | t test |
|---|---|---|
| Obese (16)/Lean (17) | 3.48 | 0.017 |

Legend “Fold change” indicates fold change in RGS10 expression calculated as the ratio of mean obese expression/mean lean expression. Numbers in parentheses indicate the number of patient samples analyzed by real time PCR.

Glut 4 translocation analysis:

| Insulin | Fold Change (Mean hRGS10/ | t test |
|---|---|---|
| (nM) | Mean LacZ) (n = 3) | (hRGS10 vs LacZ) |
| 0 | 2.95 | 0.013 |
| 1 | 1.01 | 0.299 |
| 10 | 1.01 | 0.846 |

Legend “Fold Change” indicates the following ratio; (Mean % of RGS10-expressing cells scored positive for cell surface Glut 4)/(Mean % of LacZ-expressing cells scored positive for cell surface Glut 4). h” is human. “n” is the number of replicates.

These data show that increasing the levels of RGS10 in 3T3-L1 adipocytes significantly enhances basal glucose uptake in cells.

Polypeptide Domain Structure

P2RY1 contains the following domains (designated with reference to SEQ ID NO:2): 7 transmembrane receptor domains encoded by amino acids 68 to 324.

PTPLA contains the following domains (designated with reference to SEQ ID NO.8. Protein tyrosine phosphatase like protein domain encoded by amino acids 119 to 282.

CRIM1 contains the following protein domains (designated with reference to SEQ ID NO:12):

von Willebrand factor type C domain encoded by amino acids 336 to 390,

von Willebrand factor type C domain encoded by amino acids 403 to 456,

Antistasin family domain encoded by amino acids 469 to 498,

Antistasin family domain encoded by amino acids 505 to 532,

Antistasin family domain encoded by amino acids 539 to 564,

Antistasin family domain encoded by amino acids 567 to 592,

von Willebrand factor type C domain encoded by amino acids 608 to 662,

TILa domain encoded by amino acids 679 to 731,

von Willebrand factor type C domain encoded by amino acids 679 to 734,

von Willebrand factor type C domain encoded by amino acids 753 to 808,

von Willebrand factor type C domain encoded by amino acids 819 to 873.

PPP3CA contains the following protein domain (designated with reference to SEQ ID NO:16): calcineurin-like phosphoesterase domain encoded by amino acids 83 to 285.

PTPN3a contains the following protein domains (designated with reference to SEQ ID NO:22):
FERM domain encoded by amino acids 31 to 222
PDZ domain encoded by amino acids 510 to 597
Protein Tyrosine Phosphatase encoded by amino acids 670-900.

PTPN3b splice variant contains the following protein domains (designated with reference to SEQ ID NO:26):
FERM domain encoded by amino acids 31 to 222
PDZ domain encoded by amino acids 465-552
Protein Tyrosine Phosphatase encoded by amino acids 625 to 855.

DUSP3 contains the following protein domain (designated with reference to SEQ ID NO:28): Dual specificity phosphatase, catalytic domain at amino acids 29 to 176.

RGS10 contains the following protein domains (designated with reference to SEQ ID NO:32): RGS domain encoded by amino acids 27 to 142

TABLE OF SEQUENCES

| | |
|---|---|
| SEQ ID NO:1 | human P2RY1 nucleic acid sequence |

| | | |
|---|---|---|
| HUM171847 | accession:U42029 | coding sequence:47 . . . 1168 |

CCGCCTCCTACCCCTCGGAGCCGCCGCCTAAGTCGAGGAGGAGAGAATGACCGAGGTGCTGTGGCCGG
CTGTCCCCAACGGGACGGACGCTGCCTTCCTGGCCGGTCCGGGTTCGTCCTGGGGGAACAGCACGGTC
GCCTCCACTGCCGCCGTCTCCTCGTCGTTCAAATGCGCCTTGACCAAGACGGGCTTCCAGTTTTACTA
CCTGCCGGCTGTCTACATCTTGGTATTCATCATCGGCTTCCTGGGCAACAGCGTGGCCATCTGGATGT
TCGTCTTCCACATGAAGCCCTGGAGCGGCATCTCCGTGTACATGTTCAATTTGGCTCTGGCCGACTTC
TTGTACGTGCTGACTCTGCCAGCCCTGATCTTCTACTACTTCAATAAAACAGACTGGATCTTCGGGGA
TGCCATGTGTAAACTGCAGAGGTTCATCTTTCATGTGAACCTCTATGGCAGCATCTTGTTTCTGACAT
GCATCAGTGCCCACCGGTACAGCGGTGTGGTGTACCCCCTCAAGTCCCTGGGCCGGCTCAAAAAGAAG
AATGCGATCTGTATCAGCGTGCTGGTGTGGCTCATTGTGGTGGTGGCGATCTCCCCCATCCTCTTCTA
CTCAGGTACCGGGGTCCGCAAAAACAAAACCATCACCTGTTACGACACCACCTCAGACGAGTACCTGC
GAAGTTATTTCATCTACAGCATGTGCACGACCGTGGCCATGTTCTGTGTCCCCTTGGTGCTGATTCTG
GGCTGTTACGGATTAATTGTGAGAGCTTTGATTTACAAAGATCTGGACAACTCTCCTCTGAGGAGAAA
ATCGATTTACCTGGTAATCATTGTACTGACTGTTTTTGCTGTGTCTTACATCCCTTTCCATGTGATGA
AAACGATGAACTTGAGGGCCCGGCTTGATTTTCAGACCCCAGCAATGTGTGCTTTCAATGACAGGGTT
TATGCCACGTATCAGGTGACAAGAGGTCTAGCAAGTCTCAACAGTTGTGTGGACCCCATTCTCTATTT
CTTGGCGGGAGATACTTTCAGAAGGAGACTCTCCCGAGCCACAAGGAAAGCTTCTAGAAGAAGTGAGG
CAAATTTGCAATCCAAGAGTGAAGACATGACCCTCAATATTTTACCTGAGTTCAAGCAGAATGGAGAT
ACAAGCCTGTGAAGGCACAAGAATCTCCAAACACCTCTCTGTTGTAATATGGTAGGATGCTTAACAGA
ATCAAGTACTTTTCCCCTCTTTAACTTTCTAGTTTAGAAAAAAATCAAACCAAGAAAATAGTGAGTTA
AAAAAATAATAGAAGTAGAAATGCCCACATCCACACTTAGCTTGTTTGGGTTTGCTTTCACAGTCTCT
CTTCCTTCTGACTAGAAGTATGTATAATAAAACAATACTACCTAGTTAAAAAAAAAAAAAAAAAAA

| | |
|---|---|
| SEQ ID NO:2 | human P2RY1 polypeptide sequence |

protein_id:gi1147731
MTEVLWPAVPNGTDAAFLAGPGSSWGNSTVASTAAVSSSFKCALTKTGFQFYYLPAVYILVFIIGFLG
NSVAIWMFVFHMKPWSGISVYMFNLALADFLYVLTLPALIFYYFNKTDWIFGDAMCKLQRFIFHVNLY
GSILFLTCISAHRYSGVVYPLKSLGRLKKKNAICISVLVWLIVVVAISPILFYSGTGVRKNKTITCYD
TTSDEYLRSYFIYSMCTTVAMFCVPLVLILGCYGLIVRALIYKDLDNSPLRRKSIYLVIIVLTVFAVS
YIPFHVMKTMNLRARLDFQTPAMCAFNDRVYATYQVTRGLASLNSCVDPILYFLAGDTFRRRLSRATR
KASRRSEANLQSKSEDMTLNILPEFKQNGDTSL

| | |
|---|---|
| SEQ ID NO:3 | mouse PZRY1 nucleic acid sequence |

| | |
|---|---|
| accession:NM_008772 | coding sequence:32 . . . 1153 |

TCTAGTAGCTGCCTGAGTTGGAAAGAAGAGGATGACCGAGGTGCCTTGGTCGGTTGTCCCCAACGGGA
CGGATGCTGCCTTTCTGGCGGGCCTGGGCTCGCTTTGGGGAAACAGTACTGTCGCCTCAACTGCAGCA
GTTTCCTCTTCATTCCAATGTGCCCTGACCAAGACCGGTTTCCAGTTCTACTACCTGCCGGCTGTCTA
CATTTTAGTGTTCATCATAGGCTTCCTAGGCAACAGCGTGGCTATCTGGATGTTCGTTTTCCACATGA
AGCCTTGGAGCGGCATCTCCGTGTACATGTTCAATTTGGCTCTGGCTGACTTTTTGTATGTGCTCACC
CTACCAGCCCTCATCTTCTACTACTTCAACAAGACTGACTGGATCTTCGGGGATGCTATGTGCAAGCT
GCAGAGATTCATCTTCCACGTAAATCTCTATGGTAGCATCTTGTTCCTCACCTGCATCAGCGCACACA
GGTACAGTGGCGTGGTGTACCCTCTCAAGTCTCTGGGCAGGCTCAAGAAGAAGAATGCCATTTATGTC
AGCGTGCTGGTGTGGCTCATTGTGGTGGTGGCCATCTCCCCTATTCTCTTCTACTCTGGCACTGGGAC
TCGGAAAAACAAAACTGTCACCTGCTATGACACCACGTCCAATGATTACCTGCGAAGTTATTTCATCT
ACAGTATGTGCACGACTGTGGCCATGTTCTGCATCCCTTTGGTGCTGATCTTGGGCTGTTATGGATTA
ATTGTTAAAGCTTTGATTTACAATGACCTGGACAACTCTCCGCTCCGGAGGAAATCCATTTACCTGGT
GATAATTGTCCTGACGGTGTTTGCTGTGTCTTATATCCCTTTCCATGTGATGAAAACGATGAATTTGC
GAGCACGGTTGGATTTCCAGACCCCAGAAATGTGTGATTTCAACGACAGGGTTTATGCCACTTATCAG
GTAACAAGAGGTCTAGCAAGTCTCAACAGCTGTGTGGACCCCATTCTTTATTTCTTGGCTGGAGATAC
ATTCAGAAGGAGACTGTCCCGAGCCACCAGGAAAGCTTCCAGGAGGAGTGAGGCCAATTTACAATCCA
AGAGTGAAGAAATGACTCTCAATATTTTGTCTGAGTTCAAGCAGAATGGAGACACGAGTTTGTGAAGG
CACGAGATCCTAGCTCCTGAGTTTTGTAACATGGTCACAAGACATCCCTGAGATGATCTATGCATACA
CAGGTCAAGAAGAAGCAACACTCTATTGACCAAAAGACCAGTGTGTGCCACCTGAGTGAGGGTTTTCC
T

| | |
|---|---|
| SEQ ID NO:4 | mouse P2RY1 polypeptide sequence |

accession:gi6679193
MTEVPWSVVPNGTDAAFLAGLGSLWGNSTVASTAAVSSSFQCALTKTGFQFYYLPAVYILVFIIGFLG
NSVAIWMFVFHMKPWSGISVYMFNLALADFLYVLTLPALIFYYFNKTDWIFGDAMCKLQRFIFHVNLY
GSILFLTCISAHRYSGVVYPLKSLGRLKKKNAIYVSVLVWLIVVVAISPILFYSGTGTRKNKTVTCYD
TTSNDYLRSYFIYSMCTTVAMFCIPLVLILGCYGLIVKALIYNDLDNSPLRRKSIYLVIIVLTVFAVS
YIPFHVMKTMNLRARLDFQTPEMCDFNDRVYATYQVTRGLASLNSCVDPILYFLAGDTFRRRLSRATR
KASRRSEANLQSKSEEMTLNILSEFKQNGDTSL

| | |
|---|---|
| SEQ ID NO:5 | rat P2RY1 nucleic acid sequence |

| | |
|---|---|
| accession:U22830 | coding sequence:620 . . . 1741 |

GAATTCGCCGCCGCCTGACTTCATGGACTGCGAGCGGGGTGAAGGGTAGCTCCAGGGTCCGCGGCCAA
GAGCTCTCCCAAGGCTGAGTGCGGGCTGAGAATGCGGCCGGAAGAAGAGTCGCGGCGTCCCCGGTGAC
GGTCCGGAGTTGCAAGGTAGGGAGTAGGCAGATGGCCAGGCAAAGTTGATCGGAGCGAGGAGGTGGTT
GGCCTGCGCCTTCGGCCGCTGAGAACCGCGGAGCTACTTGGACTTGACCCTACTCGCCCCAGCGCTTT
GAGCAGCGCCTATCTGTCGCCCTGGCGGGAGCGCACTTGCAAACTTGGTGGAGGTGCCCTGCGCGTCC
TGTTGTGTAACCTCCGTGCCGCCAGCTGGACCGGGAAGTTGCCCGCCCGTCCTTCAGCTTGGATCGGT
CGCAGCCCCGGGCGAATTTCATGGCCCCGCGACAAACGCGCGGCCAGAGCCGGTGTGGGCGAGCCCCC
GCGCGCTCCGACCCGTAGGGAACTCCCGCACGCGGTCCCTGCCCCTGGCCCGCGCCTCCGATGCGCGC
TGAGCCTCTCGCCAGCAGCTGCCCTCTCGTCGCGGTCTGTCCTTTCGAGTAGCTGCCTGAGTTGGAAA
GAAGAGGATGACCGAGGTGCCTTGGTCGGCTGTCCCCAACGGGACAGATGCTGCCTTCCTGGCTGGCC
TGGGCTCCCTTTGGGGAAACAGTACAATCGCCTCGACTGCAGCAGTTTCCTCTTCATTCCGATGTGCC
CTGATCAAGACCGGCTTCCAGTTCTACTACCTGCCTGCGGTCTACATCTTAGTGTTCATCATAGGCTT
CCTTGGCAACAGCGTGGCAATCTGGATGTTTGTTTTCCACATGAAGCCTTGGAGCGGCATCTCGGTGT
ACATGTTCAATTTGGCTCTGGCCGACTTTTTGTATGTGCTCACCCTACCAGCTCTCATCTTCTACTAC
TTCAACAAGACTGACTGGATCTTCGGGGATGTTATGTGCAAGCTGCAGAGGTTCATCTTCCATGTAAA
CCTCTATGGCAGCATCTTGTTCCTCACCTGCATCAGTGCACACAGGTACAGTGGCGTGGTGTACCCTC
TCAAGTCTCTGGGCAGGCTCAAGAAGAAGAATGCCATTTATGTCAGTGTGCTGGTATGGCTCATTGTG
GTGGTGGCCATCTCCCCCATTCTCTTCTACTCTGGCACTGGGATTCGGAAAAACAAAACTGTCACCTG
CTACGACTCCACGTCAGATGAGTACCTGCGAAGTTATTTCATCTACAGTATGTGCACGACTGTGGCCA
TGTTCTGCATCCCCCTGGTGCTGATCTTGGGCTGTTATGGATTAATTGTTAGAGCTTTGATCTACAAA
GATCTGGACAACTCTCCTCTCCGGAGGAAATCCATTTACCTGGTGATAATTGTCCTGACGGTGTTTGC
TGTGTCTTACATCCCTTTCCATGTGATGAAAACGATGAATTTGAGGGCACGGCTGGATTTCCAGACCC
CAGAAATGTGTGATTTCAACGACAGGGTTTATGCCACCTATCAGGTAACAAGAGGTCTAGCAAGTCTC
AACAGCTGTGTGGACCCCATTCTTTATTTCTTGGCTGGAGATACATTCAGAAGGAGACTGTCCCGAGC
CACCAGGAAAGCTTCCAGGAGGAGTGAGGCCAATTTACAATCCAAGAGTGAAGAAATGACTCTCAACA
TTTTGTCTGAGTTCAAGCAGAACGGAGACACAAGTTTGTGAAGGCAGGAGATCCAAGCTCCTCAGTTT
GTAACATGGTCACAAGAAGTCCCTGAGATGACCTGATGCACAGGTCAAGAAGAAGCAACATTCTATTG
GCCAAAAGACCAGTTTGTGCCACATGAGTAGGTGTTTTCATATAATGACAGTTATGTTGACATTTTGT
TGAAAGACACCTGAAACTGTAAGATGTAGCAAGTGACTCTTCACACTACAAAGATGTCTTCATTCCAA
GCTCAAGGGTTTGTTTTCCTTGTTGCTATCTGCTGTTTATTCCTTACAAAGTACTGTCTCGGACTCTT
TGATGGTTGCTTTGAACCTGTATTGAACGGAAATGATAAATTAAGAAGAAACTTAGCACACAAGACAG
AATTCAAAATTAAAACCGTGATGTGACCACTGAGAAGACATTTGGAAGCCCATTCTTCATGAACAGAA
AAAGGCGACAGGACACCAACTGTTGATGCCATAGGTGCTGTGGTAAGTCTTGAAAAGCAGTGAGAACT
GTTTGGAATCTGCTTCTGTGCTTCCTAGGTCTCTCCAGTCTAGTCCTTGGAGAAAGTTCTCAATCTCC
ACCGACTGTGGAACGGACAAGTTGAAACCTGTAAATCTCTGCTCACCCAACCAGGACACTAATCCATC
TTGATATGACAAATATCAGTACTCAGTAGCAACTAGAATTCAATTAGTGACAGGACATAGACAGTGTG
ATACTAGTAAATGTGTATTGAAGCTTTTCTTCCATTGGTCGAATGTAATTATTGAAATAGTATTTATT
AGAAGAGGTTTATAGGCCTTCAGATGTACAGATTGTAAAACTTCCTTGAAGATACAACATACTGGTGT
AAACTCTATTTATTGACCAGTGCATAATGCTTCAAATGATCACATGTATATTTTACAGTATATATAGC
TAGATAAAAATGAGAATTATATTTAAGAGTTTTTTTTCCAGATATTAGTATCTTTGAACCTTTAGGAC
CAGTTTGTATTTGAATCTGAAAAGATGAAAATGTGTTTTCTAACAAGGGTTTTAATTTTCCCTAGATT
TGAATTTGCATGCTTTCCTTTGTGTGGGTTTATTGCTACCCAGATATGATTTGGCTGTTAGACCATGG
TAAGATTTATAACAAGCATAGGATATTTTAGACAGGTTATGTATATGTACATTATATATGTTTATACA
ATAGAAAGTTTGTGTTGTCTTTTCAGAAAGTTTAGAGTATTGCCCTAACTATGATGCAGCTTCCACTG
CCAAAGGCTAATGGCTTATCTCCTATTCAAAATTGATTTAGATGTCTAGTGGGGTTGGGTAGTGTAGA
TACAATGTGAGAATGTAAAAGACAATCTTTAGTGATGCAGGGTGATAAAGATGCAGCTGATAGTTTTC
TGCCAGATCTTGAAGCTTTACAATGTATTTTTTTCTTAAGGCCACATAGTACAATCTTCTTTGCGGCC
GCGAATTC

| | |
|---|---|
| SEQ ID NO:6 | rat P2RY1 polypeptide sequence |

accession:gi1352695
MTEVPWSAVPNGTDAAFLAGLGSLWGNSTIASTAAVSSSFRCALIKTGFQFYYLPAVYILVFIIGFLG
NSVAIWMFVFHMKPWSGISVYMFNLALADFLYVLTLPALIFYYFNKTDWIFGDVMCKLQRFIFHVNLY
GSILFLTCISAHRYSGVVYPLKSLGRLKKKNAIYVSVLVWLIVVVAISPILFYSGTGIRKNKTVTCYD
STSDEYLRSYFIYSMCTTVAMFCIPLVLILGCYGLIVRALIYKDLDNSPLRRKSIYLVIIVLTVFAVS
YIPFHVMKTMNLRARLDFQTPEMCDFNDRVYATYQVTRGLASLNSCVDPILYFLAGDTFRRRLSRATR
KASRRSEANLQSKSEEMTLNILSEFKQNGDTSL

| | |
|---|---|
| SEQ ID NO:7 | human PTPLA nucleic acid sequence |

| | | |
|---|---|---|
| HUM178260 | accession:AF114494 | coding sequence:49 . . . 915 |

TATACACAGCCCAGCCAGCACCCAGGTCCTTCCATCCTTCTGCTGCACATGGGGCGCCTGACGGAAGC
GGCGGCAGCGGGCAGCGGCTCTCGGGCTGCAGGCTGGGCAGGGTCCCCTCCCACGCTCCTGCCGCTGT
CTCCCACGTCCCCCAGGTGCGCGGCCACCATGGCGTCCAGCGACGAGGACGGCACCAACGGCGGCGCC
TCGGAGGCCGGCGAGGACCGGGAGGCTCCCGGCAAGCGGAGGCGCCTGGGGTTGTTGGCCACCGCCTG
GCTCACCTTCTACAACATCGCCATGACCGCGGGGTGGTTGGTTCTAGCTATTGCCATGGTACGTTTTT
ATATGGAAAAGGGAACACACAGAGGGTTATATAAAAGTATTCAGAAGACACTTAAATTTTTCCAAACA
TTTGCCTTGCTTGAGATAGTTCACTGTTTAATTGGAATTGTACCTACTTCTGTGATTGTGACTGGGGT
CCAAGTGAGCTCAAGAATCTTTATGGTGTGGCTCATTACTCACAGTATAAAACCAATCCAGAATGAAG
AGAGTGTGGTGCTTTTTCTGGTCGCGTGGACTGTGACAGAGATCACTCGCTATTCCTTCTACACATTC
AGCCTTCTTGACCACTTGCCATACTTCATTAAATGGGCCAGATATAATTTTTTTATCATCTTATATCC
TGTTGGAGTTGCTGGTGAACTTCTTACAATATACGCTGCCTTGCCGTATGTGAAGAAAACAGGAATGT
TTTCAATAAGACTTCCTAACAAATACAATGTCTCTTTTGACTACTATTATTTTCTTCTTATAACCATG
GCATCATATATACCTTTGTTTCCACAACTCTATTTTCATATGTTACGTCAAAGAAGAAAGGTGCTTCA
TGGAGAGGTGATTGTAGAAAAGGATGATTAAATGATCTCTGCAAACAAGGTGCTTTTTCCAGAATAAC
CAAGATTACCTGAGTCCAAGTTTTAATAACAAGAATAAACAACTTTGTGAA

| | |
|---|---|
| SEQ ID NO:8 | human PTPLA polypeptide sequence |

protein_id:gi6650693
MGRLTEAAAAGSGSRAAGWAGSPPTLLPLSPTSPRCAATMASSDEDGTNGGASEAGEDREAPGKRRRL
GLLATAWLTFYNIAMTAGWLVLAIAMVRFYMEKGTHRGLYKSIQKTLKFFQTFALLEIVHCLIGIVPT
SVIVTGVQVSSRIFMVWLITHSIKPIQNEESVVLFLVAWTVTEITRYSFYTFSLLDHLPYFIKWARYN
FFIILYPVGVAGELLTIYAALPYVKKTGMFSIRLPNKYNVSFDYYYFLLITMASYIPLFPQLYFHMLR
QRRKVLHGEVIVEKDD

| | |
|---|---|
| SEQ ID NO:9 | mouse PTPLA nucleic acid sequence |

| | |
|---|---|
| accession:NM_013935 | coding sequence:117 . . . 962 |

GGACTTTATTCAAACCGTTGCCAAATGTGAGGCGGAGGGAGGTCTCACTCTCCAATAGGCATCAAGGA
CAGAGTTTAAGGATGTTTTGGGGACAAAGAAGACACTGCCCTTGAGGTATGGGGAAAGGTGATTGGAG
GCAGGGCAGAGTAGAGATGCCTTGTGCACACGTGAGCAGACTTCATAAGACATGCGTACAGGTCCGCG
TGCGCGTCACCATGGCGTCCAGTGAGGAGGACGGCACCAACGGCGCCTCCGAGGCCAGCGACGAGAAG
GAAGCCGCCGGCAAGCGGAGACGCCTAGGCTTACTGGCCACCGCCTGGCTCACCTTCTACAATATCGC
CATGACGGCCGGGTGGTTGGTTCTTGCTATTGCTATGGTACGCTTTTATATGGAAAAAGGAACACACA
GAGGTTTATATAAAAGCATTCAGAAGACACTTAAGTTTTTCCAAACATTTGCTTTGCTTGAGGTAGTC
CATTGTCTGATCGGAATTGTACCCACTTCTGTGCTTGTGACTGGGGTCCAAGTGAGCTCAAGAATCTT
CATGGTGTGGCTCATTACTCACAGTATAAAACCCATCCAGAATGAAGAGAGCGTGGTGCTTTTTCTGG
TCTCCTGGACTGTGACCGAGATCACTCGCTATTCCTTCTACACATTCAGTCTCCTCGACCACTTGCCG
CACTTCATTAAATGGGCCAGATACAATTTGTTTATCATCTTATATCCCGTTGGAGTTGCTGGGGAACT
TCTCACAATATACGCCGCCTTGCCTTACGTAAAGAAGTCAGGAATGTTCTCAGTACGGCTTCCCAACA
AGTACAATGTTTCTTTTGACTACTACTATTTTCTTCTCATAACCATGGCCTCCTATATACCGTTGTTT
CCTCAGCTCTATTTTCATATGTTACGTCAGAGAAGAAAGGTGCTCCACGGGGAGGTGATCGCGGAGAA
GGACGATTAAGTGGTTCCCACACACAAGGTGCTCTTTTCAGAAAAACCGGATTACTTGAGTCCAAGTT
TTAATAATAAGAATAAACGACTTCATGAAATACCAAAAAAAAAAAAAAAAAAAAAAAAA

| | |
|---|---|
| SEQ ID NO:10 | mouse PTPLA polypeptide sequence |

| | |
|---|---|
| Protein sequence | accession:gi7305421 |

MGKGDWRQGRVEMPCAHVSRLHKTCVQVRVRVTMASSEEDGTNGASEASDEKEAAGKRRRLGLLATAW
LTFYNIAMTAGWLVLAIAMVRFYMEKGTHRGLYKSIQKTLKFFQTFALLEVVHCLIGIVPTSVLVTGV
QVSSRIFMVWLITHSIKPIQNEESVVLFLVSWTVTEITRYSFYTFSLLDHLPHFIKWARYNLFIILYP
VGVAGELLTIYAALPYVKKSGMFSVRLPNKYNVSFDYYYFLLITMASYIPLFPQLYFHMLRQRRKVLH
GEVIAEKDD

| | |
|---|---|
| SEQ ID NO:11 | human CRIM1 nucleic acid sequence |

| | | |
|---|---|---|
| HUM179528 | accession:AF167706 | Coding sequence:40 . . . 3150 |

GGCCCGGCTGCGAGGAGGAGGCGGCGGCGGCGCAGGAGGATGTACTTGGTGGCGGGGGACAGGGGGTT
GGCCGGCTGCGGGCACCTCCTGGTCTCGCTGCTGGGGCTGCTGCTGCTGCTGGCGCGCTCCGGCACCC
GGGCGCTGGTCTGCCTGCCCTGTGACGAGTCCAAGTGCGAGGAGCCCAGGAACTGCCCGGGGAGCATC
GTGCAGGGCGTCTGCGGCTGCTGCTACACGTGCGCCAGCCAGAGGAACGAGAGCTGCGGCGGCACCTT
CGGGATTTACGGAACCTGCGACCGGGGGCTGCGTTGTGTCATCCGCCCCCCGCTCAATGGCGACTCCC
TCACCGAGTACGAAGCGGGCGTTTGCGAAGATGAGAACTGGACTGATGACCAACTGCTTGGTTTTAAA
CCATGCAATGAAAACCTTATTGCTGGCTGCAATATAATCAATGGGAAATGTGAATGTAACACCATTCG
AACCTGCAGCAATCCCTTTGAGTTTCCAAGTCAGGATATGTGCCTTTCAGCTTTAAAGAGAATTGAAG
AAGAGAAGCCAGATTGCTCCAAGGCCCGCTGTGAAGTCCAGTTCTCTCCACGTTGTCCTGAAGATTCT
GTTCTGATCGAGGGTTATGCTCCTCCTGGGGAGTGCTGTCCCTTACCCAGCCGCTGCGTGTGCAACCC
CGCAGGCTGTCTGCGCAAAGTCTGCCAGCCGGGAAACCTGAACATACTAGTGTCAAAAGCCTCAGGGA
AGCCGGGAGAGTGCTGTGACCTCTATGAGTGCAAACCAGTTTTCGGCGTGGACTGCAGGACTGTGGAA
TGCCCTCCTGTTCAGCAGACCGCGTGTCCCCCGGACAGCTATGAAACTCAAGTCAGACTAACTGCAGA
TGGTTGCTGTACTTTGCCAACAAGATGCGAGTGTCTCTCTGGCTTATGTGGTTTCCCCGTGTGTGAGG
TGGGATCCACTCCCCGCATAGTCTCTCGTGGCGATGGGACACCTGGAAAGTGCTGTGATGTCTTTGAA
TGTGTTAATGATACAAAGCCAGCCTGCGTATTTAACAATGTGGAATATTATGATGGAGACATGTTTCG
AATGGACAACTGTCGGTTCTGTCGATGCCAAGGGGGCGTTGCCATCTGCTTCACTGCCCAGTGTGGTG
AGATAAACTGCGAGAGGTACTACGTGCCCGAAGGAGAGTGCTGCCCAGTGTGTGAAGATCCAGTGTAT
CCTTTTAATAATCCCGCTGGCTGCTATGCCAATGGCCTGATCCTTGCCCACGGAGACCGGTGGCGGGA
AGACGACTGCACATTCTGCCAGTGCGTCAACGGTGAACGCCACTGCGTTGCGACCGTCTGCGGACAGA
CCTGCACAAACCCTGTGAAAGTGCCTGGGGAGTGTTGCCCTGTGTGCGAAGAACCAACCATCATCACA
GTTGATCCACCTGCATGTGGGGAGTTATCAAACTGCACTCTGACAGGGAAGGACTGCATTAATGGTTT
CAAACGCGATCACAATGGTTGTCGGACCTGTCAGTGCATAAACACCGAGGAACTATGTTCAGAACGTA
AACAAGGCTGCACCTTGAACTGTCCCTTCGGTTTCCTTACTGATGCCCAAAACTGTGAGATCTGTGAG
TGCCGCCCAAGGCCCAAGAAGTGCAGACCCATAATCTGTGACAAGTATTGTCCACTTGGATTGCTGAA
GAATAAGCACGGCTGTGACATCTGTCGCTGTAAGAAATGTCCAGAGCTCTCATGCAGTAAGATCTGCC
CCTTGGGTTTCCAGCAGGACAGTCACGGCTGTCTTATCTGCAAGTGCAGAGAGGCCTCTGCTTCAGCT
GGGCCACCCATCCTGTCGGGCACTTGTCTCACCGTGGATGGTCATCATCATAAAAATGAGGAGAGCTG
GCACGATGGGTGCCGGGAATGCTACTGTCTCAATGGACGGGAAATGTGTGCCCTGATCACCTGCCCGG
TGCCTGCCTGTGGCAACCCCACCATTCACCCTGGACAGTGCTGCCCATCATGTGCAGATGACTTTGTG
GTGCAGAAGCCAGAGCTCAGTACTCCCTCCATTTGCCACGCCCCTGGAGGAGAATACTTTGTGGAAGG
AGAAACGTGGAACATTGACTCCTGTACTCAGTGCACCTGCCACAGCGGACGGGTGCTGTGTGAGACAG
AGGTGTGCCCACCGCTGCTCTGCCAGAACCCCTCACGCACCCAGGATTCCTGCTGCCCACAGTGTACA
GATCAACCTTTTCGGCCTTCCTTGTCCCGCAATAACAGCGTACCTAATTACTGCAAAAATGATGAAGG
GGATATATTCCTGGCAGCTGAGTCCTGGAAGCCTGACGTTTGTACCAGCTGCATCTGCATTGATAGCG
TAATTAGCTGTTTCTCTGAGTCCTGCCCTTCTGTATCCTGTGAAAGACCTGTCTTGAGAAAAGGCCAG
TGTTGTCCCTACTGCATAGAAGACACAATTCCAAAGAAGGTGGTGTGCCACTTCAGTGGGAAGGCCTA
TGCCGACGAGGAGCGGTGGGACCTTGACAGCTGCACCCACTGCTACTGCCTGCAGGGCCAGACCCTCT
GCTCGACCGTCAGCTGCCCCCCTCTGCCCTGTGTTGAGCCCATCAACGTGGAAGGAAGTTGCTGCCCA
ATGTGTCCAGAAATGTATGTCCCAGAACCAACCAATATACCCATTGAGAAGACAAACCATCGAGGAGA
GGTTGACCTGGAGGTTCCCCTGTGGCCCACGCCTAGTGAAAATGATATCGTCCATCTCCCTAGAGATA
TGGGTCACCTCCAGGTAGATTACAGAGATAACAGGCTGCACCCAAGTGAAGATTCTTCACTGGACTCC
ATTGCCTCAGTTGTGGTTCCCATAATTATATGCCTCTCTATTATAATAGCATTCCTATTCATCAATCA
GAAGAAACAGTGGATACCACTGCTTTGCTGGTATCGAACACCAACTAAGCCTTCTTCCTTAAATAATC
AGCTAGTATCTGTGGACTGCAAGAAAGGAACCAGAGTCCAGGTGGACAGTTCCCAGAGAATGCTAAGA
ATTGCAGAACCAGATGCAAGATTCAGTGGCTTCTACAGCATGCAAAAACAGAACCATCTACAGGCAGA
CAATTTCTACCAAACAGTGTGAAGAAAGGCAACTAGGATGAGGTTTCAAAAGACGGAAGACGACTAAA
TCTGCTCTAAAAAGTAAACTAGAATTTGTGCACTTGCTTAGTGGATTGTATTGGATTGTGACTTGATG
TACAGCGCTAAGACCTTACTGGGATGGGCTCTGTCTACAGCAATGTGCAGAACAAGCATTCCCACTTT
TCCTCAAGATAACTGACCAAGTGTTTTCTTAGAACCAAAGTTTTTAAAGTTGCTAAGATATATTTGCC
TGTAAGATAGCTGTAGAGATATTTGGGGTGGGGACAGTGAGTTTGGATGGGGAAATGGGTGGGAGGGT
GGTGTTGGGAAGAAAAATTGGTCAGCTTGGCTCGGGGAGAAACCTGGTAACATAAAAGCAGTTCAGTG
GCCCAGAGGTTATTTTTTTCCTATTGCTCTGAAGACTGCACTGGTTGCTGCAAAGCTCAGGCCTGAAT
GAGCAGGAAACAAAAAAGGCCTTGCGACCCAGCTGCCATAACCACCTTAGAACTACCAGACGAGCACA
TCAGAACCCTTTGACAGCCATCCCAGGTCTAAAGCCACAAGTTTCTTTTCTATACAGTCACAACTGCA
GTAGGCAGTGAGGAAGCCAGAGAAATGCGATAGCGGCATTTCTCTAAAGCGGGTTATTAAGGATATAT
ACAGTTACACTTTTTGCTGCTTTTATTTTCTTCCAAGCCAATCAATCAGCCAGTTCCTAGCAGAGTCA
GCACATGAACAAGATCTAAGTCATTTCTTGATGTGAGCACTGGAGCTTTTTTTTTTTTACAACGTGAC
AGGAAGAGGAGGGAGAGGGTGACGAACACCAGGCATTTCCAGGGGCTATATTTCACTGTTTGTTGTTG
CTTTGTTCTGTTATATTGTTGGTTGTTCATAGTTTTTGTTGAAGCTCTAGCTTAAGAAGAAACTTTTT
TTAAAAAGACTGTTTGGGGATTCTTTTTCCTTATTATATACTGATTCTACAAAATAGAAACTACTTCA
TTTTAATTGTATATTATTCAAGCACCTTTGTTGAAGCTCAAAAAAAATGATGCCTCTTTAAACTTTAG
CAATTATAGGAGTATTTATGTAACTATCTTATGCTTCAAAAAACAAAAGTATTTGTGTGCATGTGTAT
ATAATATATATATATACATATATATTTATACACATACAATTTATGTTTTCCTGTTGAATGTATTTTTA
TGAGATTTTAACCAGAACAAAGGCAGATAAACAGGCATTCCATAGCAGTGCTTTTGATCACTTACAAA
TTTTTTGAATAACACAAAATCTCATTCTACCTGCAGTTTAATTGGAAAGATGTGTGTGTGAGAGTATG
TATGTGTGTGTGTGTGTGTGTGTGTGTGCGCGCGCACGCACGCCTTGAGCAGTCAGCATTGCACCTGC
TATGGAGAAGGGTATTCCTTTATTAAAATCTTCCTCATTTGGATTTGCTTTCAGTTGGTTTTCAATTT
GCTCACTGGCCAGAGACATTGATGGCAGTTCTTATCTGCATCACTAATCAGCTCCTGGATTTTTTTTT
TTTTTTTTTCAAACAATGGTTTGAAACAACTACTGGAATATTGTCCACAATAAGCTGGAAGTTTGTTG
TAGTATGCCTCAAATATAACTGACTGTATACTATAGTGGTAACTTTTCAAACAGCCCTTAGCACTTTT
ATACTAATTAACCCATTTGTGCATTGAGTTTTCTTTTAAAAATGCTTGTTGTGAAAGACACAGATACC
CAGTATGCTTAACGTGAAAAGAAAATGTGTTCTGTTTTGTAAAGGAACTTTCAAGTATTGTTGTAAAT
ACTTGGACAGAGGTTGCTGAACTTTAAAAAAAATTAATTTATTATTATAATGACCTAATTTATTAATC
TGAAGATTAACCATTTTTTTGTCTTAGAATATCAAAAAGAAAAAGAAAAAGGTGTTCTAGCTGTTTGC
ATCAAAGGAAAAAAAGATTTATTATCAAGGGGCAATATTTTTATCTTTTCCAAAATAAATTTGTTAAT
GATACATTACAAAAATAGATTGACATCAGCCTGATTAGTATAAATTTTGTTGGTAATTAATCCATTCC
TGGCATAAAAAGTCTTTATCAAAAAAAATTGTAGATGCTTGCTTTTTGTTTTTTCAATCATGGCCATA
TTATGAAAATACTAACAGGATATAGGACAAGGTGTAAATTTTTTTATTATTATTTTAAAGATATGATT
TATCCTGAGTGCTGTATCTATTACTCTTTTACTTTGGTTCCTGTTGTGCTCTTGTAAAAGAAAAATAT
AATTTCCTGAAGAATAAAATAGATATATGGCACTTGGAGTGCATCATAGTTCTACAGTTTGTTTTTGT
TTTCTTCAAAAAAGCTGTAAGAGAATTATCTGCAACTTGATTCTTGGCAGGAAATAAACATTTTGAGT
TGAAATCAAAAAAAAAAAAAAAAAA

| | |
|---|---|
| SEQ ID NO:12 | human CRIM1 polypeptide sequence |

| | |
|---|---|
| Protein sequence | protein_id:gi6979311 |

MYLVAGDRGLAGCGHLLVSLLGLLLLLARSGTRALVCLPCDESKCEEPRNCPGSIVQGVCGCCYTCAS
QRNESCGGTFGIYGTCDRGLRCVIRPPLNGDSLTEYEAGVCEDENWTDDQLLGFKPCNENLIAGCNII
NGKCECNTIRTCSNPFEFPSQDMCLSALKRIEEEKPDCSKARCEVQFSPRCPEDSVLIEGYAPPGECC
PLPSRCVCNPAGCLRKVCQPGNLNILVSKASGKPGECCDLYECKPVFGVDCRTVECPPVQQTACPPDS
YETQVRLTADGCCTLPTRCECLSGLCGFPVCEVGSTPRIVSRGDGTPGKCCDVFECVNDTKPACVFNN
VEYYDGDMFRMDNCRFCRCQGGVAICFTAQCGEINCERYYVPEGECCPVCEDPVYPFNNPAGCYANGL
ILAHGDRWREDDCTFCQCVNGERHCVATVCGQTCTNPVKVPGECCPVCEEPTIITVDPPACGELSNCT
LTGKDCINGFKRDHNGCRTCQCINTEELCSERKQGCTLNCPFGFLTDAQNCEICECRPRPKKCRPIIC
DKYCPLGLLKNKHGCDICRCKKCPELSCSKICPLGFQQDSHGCLICKCREASASAGPPILSGTCLTVD
GHHHKNEESWHDGCRECYCLNGREMCALITCPVPACGNPTIHPGQCCPSCADDFVVQKPELSTPSICH
APGGEYFVEGETWNIDSCTQCTCHSGRVLCETEVCPPLLCQNPSRTQDSCCPQCTDQPFRPSLSRNNS
VPNYCKNDEGDIFLAAESWKPDVCTSCICIDSVISCFSESCPSVSCERPVLRKGQCCPYCIEDTIPKK
VVCHFSGKAYADEERWDLDSCTHCYCLQGQTLCSTVSCPPLPCVEPINVEGSCCPMCPEMYVPEPTNI
PIEKTNHRGEVDLEVPLWPTPSENDIVHLPRDMGHLQVDYRDNRLHPSEDSSLDSIASVVVPIIICLS
IIIAFLFINQKKQWIPLLCWYRTPTKPSSLNNQLVSVDCKKGTRVQVDSSQRMLRIAEPDARFSGFYS
MQKQNHLQADNFYQTV

| | |
|---|---|
| SEQ ID NO:13 | mouse CRIM1 nucleic acid sequence |

| | |
|---|---|
| accession:AF168680 | Coding sequence:1 . . . 3087 |

CTGGCCGGCTGCGGGCACCTCTCGGTCTCGCTGCTGGGGCTGCTGCTGCTGCTGGCGCGCTCAGGCAC
CCGGGCGCTGGTCTGCCTGCCCTGTGACGAGTCCAAGTGCGAGGAGCCTCGGAGCTGCCCAGGAAGCA
TCGTGCAGGGCGTCTGCGGCTGCTGCTACATGTGCGCCCGCCAGAGGAACGAGAGCTGCGGTGGAGCC
TATGGGCTCCATGGAGCCTGCGACCGGGGGCTGCGCTGTGTCATCCGCCCCCCGCTCAATGGCGACTC
CATCACCGAGTACGAAGTGGGCGTCTGCGAAGATGAGGACTGGGATGATGACCAGCTAATAGGTTTTG
AACCCTGCAATGAAAACCTCATCTCCGGCTGCAACATAATCAATGGGAAATGCGAATGTGGTACCATC
CGAACCTGCAACAATCCCTTTGAGTTTCCAAGGAAGGACATGTGCCTTTCAGCATTAAAGAGGATCGA
AGAAGAGAAGCCAGATTGCAGCAAGGCCCGCTGTGAAGTGCGGTTCTCTCCACGTTGCCCTGAAGATT
CCATTCTGATCGAGGGCTATGCTCCCCCCGGGGAGTGCTGTCCTTTACCCAGCCGCTGCGTGTGCGAC
CCTGCGGGCTGTCTGCGCAAAGTCTGCCAGCCAGGATACCTGAACATTCTAGTGTCCAAAGCCTCAGG
GAAGCCGGGAGAGTGTTGTGACCTCTATGAGTGTAAACCAGTTTTCAGCGTGGACTGCAGCACCGTGG
AGTGCCCCCCTGTCCAGCAGGCCGTGTGCCCCCTGGACAGCTACGAAACGCAAGTGCGGCTCACAGCG
GATGGCTGCTGTACCCTGCCAGCAAGATGCGAGTGTCTCTCTGGCTTATGTGGTTTTCCCGTTTGTGA
GGTGGGATCTACTCCCCGAATAGTCTCTCGTGGAGATGGGACACCTGGAAAGTGCTGTGATGTCTTTG
AATGTGTTAATGAAACAAAGCCAGCCTGCGTGTTCAACAGCGTGGAGTATTACGACGGAGACATGTTT
CGAATGGACAACTGTCGGTTCTGCCGATGCCAGGGGGGTGTCTCCATCTGCTTCACGGCGCAGTGTGG
GGAACTGAACTGCGAAAGATACTATGTGCCTGAGGGGGAGTGTTGCCCTGTGTGTGAAGATCCCATCT
ATCCTCTTAACAACCCTGCTGGCTGCTATGCCAATGGCCAGATCCGCGCCCACGGGGACCGGTGGCGG
GAAGATGACTGTACCTTCTGCCAGTGTATCAACGGAGAACCTCACTGCGTGGCCACGGCCTGCGGGCA
GAGCTGCATGCACCCAGTGAAAGTGCCCGGGGAGTGTTGCCCCGTGTGTGAAGAACCAACCTACATCA
CGATTGATCCACCTGCATGCGGGGAGTTGTCAAACTGTTCTCTGAAGGAGAAGGACTGCGTTTATGGC
TTCAAACTGGATCACAATGGCTGTCGAACCTGTCAGTGCAAAATCAGGGAGGAACTCTGCTTAGGCCT
CAAAAGGGCCTGCACCCTGGACTGTCCCTTTGGCTTCCTCACCGATGTGCACAACTGTGAACTCTGTC
AGTGCCGCCCACGGCCCAAGAAGTGCAGACCAACAATGTGCGACAAGTTTTGTCCGCTTGGATTCCTG
AAGAATAAGCATGGCTGTGACATCTGTCGGTGTAAGAAATGTCCAGAGCTGCCATGCAGCAAGATCTG
CCCCTTGGGCTTCCAGCAGGACAGTCATGGCTGTCTTATCTGCAAGTGCCGAGAGGTCCCTCCTTCAG
CCGGGCCACCTGTCCTGTCAGGCACATGTCTGTCCATGGATGGCCATCATCATAAGAACGAGGAGAGC
TGGCATGATGGTTGCCGGGAATGCTACTGTCACAACGGAAAGGAAATGTGCGCTCTCATCACCTGTCC
CGTGCCTGCCTGCGGCAACCCCACCATTCGTTCCGGACAGTGCTGCCCGTCTTGCACAGATGACTTTG
TAGTGCAGAAGCCGGAGCTCAGCACCCCTTCTATTTGCCACGCCCCCGGAGGAGAGTACTTTGTGGAA
GGGGAAACTTGGAACATTGACTCCTGTACACAGTGCACCTGTCACAGTGGTCGAGTGCTGTGTGAGAC
GGAGGTGTGCCCACCATTGCTCTGCCAGAACCCCTCCCGCACCCAGGACTCCTGCTGCCCACAGTGTA
CAGATGACCCTCCTCAGCCTTCCACATCCCATAATGAGAGCGTGCCTAGCTACTGCAGGAATGATGAA
GGAGATATCTTCCTGGCGGCTGAGTCCTGGAAGCCCGACGCCTGCACCAGCTGCGTGTGCGTGGATAG
CGCAATTAGCTGCTACTCTGAGTCTTGCCCTTCCGTGGCCTGTGAAAGACCTGTTTTGAGGAAAGGCC
AGTGTTGCCCCTACTGCTTAGAAGACACAATTCCAAAGAAAGTGGTGTGTCACTTCAGTGGGAAGACC
TATGCTGACGAGGAACGGTGGGATATTGACAGCTGCACRCACTGCTACTGCCTGCAAGGCCAGACCCT
CTGCTCGACCGTCAGCTGCCCACCGTTACCCTGTGCCGAGCCCATCAAGGTGGAAGGGAGTTGCTGCC
CAATGTGCCCAGAAATGTATGTGCCAGAGCCAACCAATGTACCCATTGAGAAGAAAAATCATCGTGGC
GAGATTGACCTGGAGGTCCCCATGTGGCCCACCCCAAGTGAAAATGACATCATCCATCTCCCTAGAGA
TATGGGTCACCTCCAGGTAGATTACAGAGATAATAACAGGCTGCATCCAGGCGAAGACTCATCACTGG
ACTCCATTGTCTCCGTTGTGGTTCCCATAATAATATGCCTGTCCATCATCATAGCTTTCCTGCTCATC
AACCAGAAGAAGCAGTGGGTACCACTGCTGTGCTGGTACCGGACACCAACCAAGCCTTCTTCCTTGAA
TAATCAGCTGGTATCTGTGGACTGCAAGAAAGGCACTCGAGTCCAGGTGGATGGTCCCCAGAGAATGC
TAAGAATTGCCGAACCAGATGCTCGATTCAGTGGCTTCTACAGCATGCAGAAACAGAACCATCTACAG
GCAGACAACTTCTACCAAACGGTGTGAACACGGGCAGCAGCGCCGATGAGGTCCCGAAAGACGGAGAA
AGACGAAAATCTGCTCTTTAAAGTAAACTAGGATTTGCGCACTTGCTTAGTGTCTTGTGTCAGATTGC
GACTTGGTGTCCAGCGCTAGGGGCTGGACTGGGATGGGCTCTGTCTACAGCACTGTGCAGAACAAGCA
TTCCCACTTGGCCTCAAGATAACTGACCACATGTTTTCATAGAACCAAAGTTTTAAAACTTGCTAAAG
TATATTTGCTTGTAAGATAGCTGTAGAGACATTTGGGGGAGGGGAGAAAGAGTTTGGTGGGGGAAATG
GGTTGGGAGGGTAGTGTTGGGAAGAAAAAAAAATGGTCAGCTTGGCTCGGGGAGAAAGCCAGTAAAAA
AATAAAAGCAATTTATGGCCCAGAGGAATTTCTTTTTCCTGTTACTNTCAAGACTGTTGGTTGCTGCA
AAGGTAGGGCCTGAATGAGCAGGATCACAAAAGGCCTTGCAACCGAGCTGCCATACCACACCAGACAA
GCGCATCAGAACTCTCGATAGCCATCCCAGGTCTAAAGCCACAAGTTTCTATAGAGTCACAGCCGCAG
TAGGCAGTGAGGAAGCCAGGGAGATGGAAAGCAACAGTTCTCGAAAGCGGGTTCTTGAGGATGTATTC
ACTTTTTTTGCTGCTGCTGTTTTCTTCCGTGCCAACCAGCCAGTTCCTAGGAGACACAGCAGGTTGAG
TAGGAACGAGGTCACCTCTTGACCTGACCACTGGAGCTTTGCTCGCTACGTGACAGGAAGGGCAACGG
CGAAGGACACCAGGCATTTCCAGGGGCTACACTTCATTGTTCCTTGTTGTTTTCTTCTGTGCTATCAT
TGGTTGTTCATAGTTTTGTTAAAGCTCTAGCTTAAGAAGAAACTTTTGAGAAAAAAAAAAAAAAAAAA

| | |
|---|---|
| SEQ ID NO:14 | mouse CRIM1 polypeptide sequence |

| | |
|---|---|
| Protein sequence | accession:gi6979313 |

LAGCGHLSVSLLGLLLLLARSGTRALVCLPCDESKCEEPRSCPGSIVQGVCGCCYMCARQRNESCGGA
YGLHGACDRGLRCVIRPPLNGDSITEYEVGVCEDEDWDDDQLIGFEPCNENLISGCNIINGKCECGTI
RTCNNPFEFPRKDMCLSALKRIEEEKPDCSKARCEVRFSPRCPEDSILIEGYAPPGECCPLPSRCVCD
PAGCLRKVCQPGYLNILVSKASGKPGECCDLYECKPVFSVDCSTVECPPVQQAVCPLDSYETQVRLTA
DGCCTLPARCECLSGLCGFPVCEVGSTPRIVSRGDGTPGKCCDVFECVNETKPACVFNSVEYYDGDMF
RMDNCRFCRCQGGVSICFTAQCGELNCERYYVPEGECCPVCEDPIYPLNNPAGCYANGQIRAHGDRWR
EDDCTFCQCINGEPHCVATACGQSCMHPVKVPGECCPVCEEPTYITIDPPACGELSNCSLKEKDCVYG
FKLDHNGCRTCQCKIREELCLGLKRACTLDCPFGFLTDVHNCELCQCRPRPKKCRPTMCDKFCPLGFL
KNKHGCDICRCKKCPELPCSKICPLGFQQDSHGCLICKCREVPPSAGPPVLSGTCLSMDGHHHKNEES
WHDGCRECYCHNGKEMCALITCPVPACGNPTIRSGQCCPSCTDDFVVQKPELSTPSICHAPGGEYFVE
GETWNIDSCTQCTCHSGRVLCETEVCPPLLCQNPSRTQDSCCPQCTDDPPQPSTSHNESVPSYCRNDE
GDIFLAAESWKPDACTSCVCVDSAISCYSESCPSVACERPVLRKGQCCPYCLEDTIPKKVVCHFSGKT
YADEERWDIDSCTHCYCLQGQTLCSTVSCPPLPCAEPIKVEGSCCPMCPEMYVPEPTNVPIEKKNHRG
EIDLEVPMWPTPSENDIIHLPRDMGHLQVDYRDNNRLHPGEDSSLDSIVSVVVPIIICLSIIIAFLLI
NQKKQWVPLLCWYRTPTKPSSLNNQLVSVDCKKGTRVQVDGPQRMLRIAEPDARFSGFYSMQKQNHLQ
ADNFYQTV

| | |
|---|---|
| SEQ ID NO:15 | human PPP3CA nucleic acid sequence |

| | | |
|---|---|---|
| HUM225196 | accession:L14778 | coding sequence:148 . . . 1713 |

CCAGCTCAGAGCCTAGACCTCCAGCCGAGCGGTTTGCAGCCGCGGGCGGCGGCGGCGGCGGCGGCGTT
GAGTGTCTGGCCCGCCGGTCCGGTCGGGGTGTGCAGTCGGACGGACGAGCAGCGCGTCGCTGTCCTCC
GGCAGCTGGAGATGTCCGAGCCCAAGGCAATTGATCCCAAGTTGTCGACGACCGACAGGGTGGTGAAA
GCTGTTCCATTTCCTCCAAGTCACCGGCTTACAGCAAAAGAAGTGTTTGATAATGATGGAAAACCTCG
TGTGGATATCTTAAAGGCGCATCTTATGAAGGAGGGAAGGCTGGAAGAGAGTGTTGCATTGAGAATAA
TAACAGAGGGTGCATCAATTCTTCGACAGGAAAAAAATTTGCTGGATATTGATGCGCCAGTCACTGTT
TGTGGGGACATTCATGGACAATTCTTTGATTTGATGAAGCTCTTTGAAGTCGGGGGATCTCCTGCCAA
CACTCGCTACCTCTTCTTAGGGGACTATGTTGACAGAGGGTACTTCAGTATTGAATGTGTGCTGTATT
TGTGGGCCTTGAAAATTCTCTACCCCAAAACACTGTTTTTACTTCGTGGAAATCATGAATGTAGACAT
CTAACAGAGTATTTCACATTTAAACAAGAATGTAAAATAAAGTATTCAGAACGAGTATATGATGCCTG
TATGGATGCCTTTGACTGCCTTCCCCTGGCTGCCCTGATGAACCAACAGTTCCTGTGTGTGCATGGTG
GTTTGTCTCCAGAGATTAACACTTTAGATGATATCAGAAAATTAGACCGATTCAAAGAACCACCTGCA
TATGGACCTATGTGTGATATCCTGTGGTCAGACCCCCTGGAAGATTTTGGAAATGAGAAGACTCAGGA
ACATTTCACTCACAACACAGTCAGGGGGTGTTCATACTTCTACAGTTACCCGGCTGTATGTGAATTCT
TACAGCACAATAACTTGTTATCTATACTCCGAGCCCACGAAGCCCAAGATGCAGGGTACCGCATGTAC
AGGAAAAGCCAAACAACAGGCTTCCCTTCTCTAATTACAATTTTTTCAGCACCAAATTACTTAGATGT
ATACAATAACAAAGCTGCAGTATTGAAGTATGAGAACAATGTTATGAATATCAGGCAATTCAACTGTT
CTCCTCATCCATACTGGCTTCCAAATTTCATGGATGTTTTTACTTGGTCCCTTCCATTTGTTGGGGAA
AAAGTGACTGAGATGCTGGTAAATGTCCTCAACATCTGCTCAGATGATGAACTAGGGTCAGAAGAAGA
TGGATTTGATGGTGCAACAGCTGCAGCCCGGAAAGAGGTGATAAGGAACAAGATCCGAGCAATAGGCA
AAATGGCCAGAGTGTTCTCAGTGCTCAGAGAAGAGAGTGAGAGTGTGCTGACGCTGAAAGGCTTGACC
CCAACTGGCATGCTCCCCAGCGGAGTACTTTCTGGAGGGAAGCAAACCCTGCAAAGCGCTACTGTTGA
GGCTATTGAGGCTGATGAAGCTATCAAAGGATTTTCACCACAACATAAGATCACTAGCTTCGAGGAAG
CCAAGGGCTTAGACCGAATTAATGAGAGGATGCCGCCTCGCAGAGATGCCATGCCCTCTGACGCCAAC
CTTAACTCCATCAACAAGGCTCTCACCTCAGAGACTAACGGCACGGACAGCAATGGCAGTAATAGCAG
CAATATTCAGTGACCACTTCCTGTTCACATTTTTTTTTTTTTTTTTTTTTTTTTTTTTTTTGAGCTGC
GGGGCATGATGGGGATTGCTGCATATCAGCAGTTGGATGTTCTTGCCTCTGACAGTAGCTTATTTGCT
CTGGGGGCCAGGAATTGGATTCAGTTTACACTATCATTAAAAAAGAGGGAGAGAGATAATAAACTATA
TTTTGGTGGGGATGGTGATTAAACACCTCTTTTGGGTATGCCTTTTAAAAATGCTTATAGAGAAAAAA
AATTTTAAAAAAAGAAAGCTAATGCTAGTATATACTGCAATGTTAGGGGAATGAACATGTTTTCCTAC
TGCATTGGGGACTTCTAGATAGGTTAATGAAAGGCCTTTTATTCTGTTACTGGACATGAAAACTTTGT
CTAATTTCTTACTCTATTGTACGTTTACAGTCGCAGCACTAAAAATGGATGACATCAAACATTTTTAA
CAAAATGATGATGTACAAACTAAGGACTATTTATTGATAATGTTTTGCTACTCTTGTCAGACAATGGC
TATAAACTGAATTAGGCAGTCTTAAAAAAAAA

| | |
|---|---|
| SEQ ID NO:16 | human PPP3CA polypeptide sequence |

| | |
|---|---|
| Protein sequence | protein_id:gi306477 |

MSEPKAIDPKLSTTDRVVKAVPFPPSHRLTAKEVFDNDGKPRVDILKAHLMKEGRLEESVALRIITEG
ASILRQEKNLLDIDAPVTVCGDIHGQFFDLMKLFEVGGSPANTRYLFLGDYVDRGYFSIECVLYLWAL
KILYPKTLFLLRGNHECRHLTEYFTFKQECKIKYSERVYDACMDAFDCLPLAALMNQQFLCVHGGLSP
EINTLDDIRKLDRFKEPPAYGPMCDILWSDPLEDFGNEKTQEHFTHNTVRGCSYFYSYPAVCEFLQHN
NLLSILRAHEAQDAGYRMYRKSQTTGFPSLITIFSAPNYLDVYNNKAAVLKYENNVMNIRQFNCSPHP
YWLPNFMDVFTWSLPFVGEKVTEMLVNVLNICSDDELGSEEDGFDGATAAARKEVIRNKIRAIGKMAR
VFSVLREESESVLTLKGLTPTGMLPSGVLSGGKQTLQSATVEAIEADEAIKGFSPQHKITSFEEAKGL
DRINERMPPRRDAMPSDANLNSINKALTSETNGTDSNGSNSSNIQ

| | |
|---|---|
| SEQ ID NO:17 | mouse PPP3CA nucleic acid sequence |

| | |
|---|---|
| accession:J05479 | coding sequence:76 . . . 1441 |

GCGCCGGTGCGGTCGGGGTGTGCAGTCGGACGGGACGAGCAGCGCGTCGCTGTCCCCCCCTCCCGGTG
GCTGGAGATGTCCGAGCCCAAGGCGATTGATCCCAAGTTGTCGACGACCGACAGGGTGGTGAAAGCCG
TTCCATTTCCACCAAGTCACCGGCTGACAGCAAAGGAAGTGTTTGATAATGATGGGAAACCTCGTGTG
GATATCTTAAAAGCACATCTCATGAAGGAGGGCAGGCTGGAAGAAAGTGTTGCATTGAGAATAATAAC
AGAGGGTGCTTCGATTCTCCGACAGGAAAAAAACTTGCTGGATATCGACGCACCAGTCACAGTTTGTG
GGGACATCCATGGACAATTCTTTGACTTGATGAAGCTCTTTGAAGTGGGAGGATCTCCTGCCAACACT
CGCTACCTCTTCTTAGGGGACTATGTTGACAGAGGGTACTTCAGTATCGAATGTGTGCTGTATTTGTG
GGCCTTGAAAATTCTTTACCCCAAAACACTGTTTTTACTTCGCGGAAACCATGAATGTAGGCACCTCA
CAGAGTATTTCACGTTTAAACAAGAATGTAAAATAAAGTATTCAGAACGCGTTTATGACGCCTGTATG
GATGCCTTCGACTGCCTTCCCCTGGCTGCGCTAATGAACCAGCAGTTCCTGTGTGTACACGGTGGTTT
GTCTCCAGAGATTAACACTCTAGATGACATCAGAAAATTAGACCGATTCAAAGAACCACCTGCTTATG
GGCCCATGTGTGACATCCTATGGTCAGACCCCCTGGAGGACTTTGGAAATGAGAAGACTCAGGAACAT
TTCACTCACAACACAGTCAGAGGCTGTTCGTACTTCTACAGTTACCCAGCTGTGTGTGACTTCCTGCA
GCACAATAATTTGTTGTCCATACTCCGCGCCCACGAAGCCCAGGATGCAGGGTACCGCATGTACAGGA
AAAGCCAAACAACAGGCTTCCCGTCTCTAATTACAATCTTCTCGGCACCAAATTACTTAGATGTGTAC
AATAACAAAGCTGCAGTGTTGAAGTACGAGAACAATGTGATGAACATCAGGCAGTTCAACTGCTCCCC
GCATCCGTACTGGCTCCCAAATTTCATGGATGTTTTCACCTGGTCGCTGCCATTTGTTGGGGAGAAAG
TGACTGAGATGCTGGTCAATGTTCTCAACATCTGCTCCGACGATGAACTGGGGTCAGAAGAAGATGGA
TTTGACGGAGCCACGGCCGCAGCCCGGAAGGAAGTCATCAGAAACAAGATCCGAGCAATAGGCAAAAT
GGCCAGAGTGTTCTCAGTTCTCAGAGAAGAGAGTGAGAGTGTCCTGACACTGAAGGGCCTGACCCCAA
CTGGCATGCTCCCCAGCGGAGTGCTCTCTGGCGGGAAACAGACTCTGCAAAGCGCTACTGTTGAGGCT
ATTGAGGCTGATGAAGCCATCAAAGGATTTTCACCACAACATAAGATCACTAGCTTCGAGGAGGCCAA
GGGCTTAGACCGAATTAACGAGAGGATGCCACCTCGCAGAGACGCCATGCCCTCTGACGCCAACCTTA
ACTCCATCAACAAGGCTCTCGCCTCAGAGACTAACGGCACGGACAGCAATGGCAGTAATAGCAGCAAT
ATCCAGTGACCACTTCCTGTTCACTTTTTTTTTTTTTTGAGCTGCAGGGCATGATGGGATTGCTGCAT
CTCAGCAGTTGGATGTTCTTGCCTCTGAAGGTAGCTTGTTTGCTCTGGGGGCCAGGAATTGGATTCAG
TTTACACTATCATGAAAAATAAAAATAAAAAAAGAGGGAGAGAGATAATAAACTATATTTTGGTGAGG
GTGGTGATTAAACACCTCTTTTGGGTATGCCTTTAAAAAATGCTTCTAGGGCAAAAAAGTTTTAAAAA
GAAAGCTAATGCTAGCTATACTGCAATGTTAGGGGAATGAACGCGTTTTCCTACTGCACTGGGAACTT
TTAGATAGGTTAATGAAAGGCCTTTATTCTGTTACTGGACACGAAAACTTTGTCTAATTTCTTATACT
CTATTGTACCTTTACAGTCGCAGCACTAAAATGGAAGACATCAAACATTTTTAACAGAAAAAAAAAAA
GATGTAAAAACTAACTAAGGACTATTTATTAATGATGTTTTGCTACTCCTGTCAGACAATGGCTATAA
ACTGAATTAGGCAGTCTT

| | |
|---|---|
| SEQ ID NO:18 | mouse PPP3CA polypeptide sequence |

| | |
|---|---|
| Protein sequence | accession:gi309129 |

MSEPKAIDPKLSTTDRVVKAVPFPPSHRLTAKEVFDNDGKPRVDILKAHLMKEGRLEESVALRIITEG
ASILRQEKNLLDIDAPVTVCGDIHGQFFDLMKLFEVGGSPANTRYLFLGDYVDRGYFSIECVLYLWAL
KILYPKTLFLLRGNHECRHLTEYFTFKQECKIKYSERVYDACMDAFDCLPLAALMNQQFLCVHGGLSP
EINTLDDIRKLDRFKEPPAYGPMCDILWSDPLEDFGNEKTQEHFTHNTVRGCSYFYSYPAVCDFLQHN
NLLSILRAHEAQDAGYRMYRKSQTTGFPSLITIFSAPNYLDVYNNKAAVLKYENNVMNIRQFNCSPHP
YWLPNFMDVFTWSLPFVGEKVTEMLVNVLNICSDDELGSEEDGFDGATAAARKEVIRNKIRAIGKMAR
VFSVLREESESVLTLKGLTPTGMLPSGVLSGGKQTLQSATVEAIEADEAIKGFSPQHKITSFEEAKGL
DRINERMPPRRDAMPSDANLNSINKALASETNGTDSNGSNSSNIQ

| | |
|---|---|
| SEQ ID NO:19 | rat PPP3CA nucleic acid sequence |

| | |
|---|---|
| accession:X57115 | coding sequence:271 . . . 1806 |

AGAAAATAAATAAATAACCAGCACACACGCGCAGCCCGGAGCGAGGCGCGGGGCTGGTGGCGGCGGAG
GAGGAGTGAAGGCGGCGGCGGCGGAGGAGGGACGCGCGGAGCCGGCAGTAACTTTCGAGCCAGCCCAG
AGCCCGGAGCTCCAGCCGAGCGGTTTGCAGCGCGGCGGCGGCGGCGCTGAGTGTCTGGCCCGCCGGTG
CGGTCGGGGTGTGCAGTCGGACGGGACCAGCAGCGCGTCGCTGTCCCCCCCTCCCGGTGACTGGAGAT
GTCCGAGCCCAAGGCGATTGATCCCAAGTTGTCGACTACGGACAGGGTGGTGAAAGCCGTTCCATTTC
CGCCAAGTCACCGGCTGACAGCAAAGGAAGTGTTTGATAACGATGGGAAGCCTCGTGTGGATATCTTA
AAAGCACATCTCATGAAGGAAGGCAGGCTGGAAGAAAGTGTCGCGTTGAGAATAATAACAGAGGGTGC
TTCGATTCTCCGACAGGAAAAAAACTTGCTGGATATTGATGCCCCAGTCACAGTTTGCGGGGACATCC
ATGGACAATTCTTTGACTTGATGAAGCTCTTTGAAGTGGGAGGATCTCCTGCCAACACTCGCTACCTC
TTCTTAGGGGACTATGTTGACAGAGGGTACTTCAGTATCGAATGTGTGCTGTATTTGTGGGCCTTGAA
AATTCTTTACCCCAAAACACTGTTTTTACTTCGTGGAAACCATGAATGTAGGCACCTAACAGAGTATT
TCACGTTTAAACAAGAATGTAAAATAAAGTATTCAGAACGCGTTTATGACGCCTGTATGGATGCCTTC
GACTGCCTTCCCCTGGCTGCGCTGATGAACCAACAATTCCTGTGTGTACACGGTGGTTTGTCTCCAGA
GATTAACACTCTAGATGACATCAGAAAATTAGACCGATTCAAAGAACCACCTGCTTATGGGCCTATGT
GTGACATCTTGTGGTCAGACCCCCTGGAGGACTTTGGAAATGAGAAGACTCAGGAACATTTCACTCAC
AACACAGTCAGGGGTTGTTCGTACTTCTACAGTTACCCGGCTGTATGTGACTTCCTGCAGCACAATAA
TTTGTTGTCCATACTCCGAGCCCACGAAGCCCAGGACGCAGGGTACCGCATGTACAGGAAAAGCCAAA
CAACTGGCTTCCCGTCTCTAATTACGATCTTCTCGGCACCAAATTACTTAGATGTGTACAATAATAAA
GCTGCAGTGTTGAAGTACGAGAACAACGTGATGAACATCAGGCAGTTCAACTGCTCCCCCCATCCGTA
CTGGCTCCCAAATTTCATGGATGTTTTCACCTGGTCGCTGCCATTTGTTGGGGAGAAAGTGACTGAGA
TGCTGGTAAACGTCCTGAACATCTGCTCAGATGATGAACTGGGGTCAGAAGAAGATGGATTTGACGGA
GCCACGGCTGCAGCCCGGAAGGAGGTCATCAGGAACAAGATCCGAGCAATAGGCAAAATGGCCAGAGT
ATTCTCAGTTCTCAGAGAAGAGAGTGAGAGCGTTCTAACTCTGAAGGGCCTGACCCCGACTGGCATGC
TCCCCAGCGGAGTGCTCTCTGGCGGGAAACAAACTCTGCAAAGCGCCATCAAAGGATTCTCACCACAA
CATAAGATTACCAGCTTCGAGGAGGCCAAGGGCTTAGACCGAATTAACGAGAGGATGCCGCCTCGCAG
AGACGCCATGCCTTCCGACGCCAACCTTAACTCCATCAACAAGGCTCTCGCCTCAGAGACTAACGGCA
CAGACAGCAACGGCAGTAATAGCAGCAATATTCAGTGACCACTTCCTGTTCACTTTTTTTTTTTGAGC
TGCAGGCATGATGGGTTTGCTGCATCTCAGCAGTTGGTGTTCTTGCCTCTGACGGTAGCTTGTTTGCT
GCTGGGGGGGCCAGGAATTGGATTCAGTTTACACTATCATGAAAAAAAAAAAAGAGGGAGAGAGAGAG
AGATAATAAAACTATATTTTGGTGAGGGTGGTGATTAAACACCTCTTTTGGGTATGCCTTTAAAAATG
CTTCTAGGAAAAAAAAAGTTTTAAAAAGAAAGCTAATGCTAGTCTATACTTCAATGTTAGGGGAATGA
ACACGTTTTCCTAGCGCACTGGGGACTTTTAGATAGGTTAATGAAAGGCCTTTTATTCTGTTACTGGA
CACGAAAACTTTGTCTAATTTCTTATACTCTATTGTACGTTTACAGTCGCAGCACTAAAAATGGATGA
CATCAAACATTTTTAAACAGAAAAAAAAAGATGTGCGGGCTAAATAACCACTATTTATTGATAATGTT
TTGCTACTCTTGTCAGACAATGGCTATAAACTGAATTAGGCCGAATTC

| | |
|---|---|
| SEQ ID NO:20 | rat PPP3CA polypeptide sequence |

| | |
|---|---|
| Protein sequence | accession:gi4584820 |

MSEPKAIDPKLSTTDRVVKAVPFPPSHRLTAKEVFDNDGKPRVDILKAHLMKEGRLEESVALRIITEG
ASILRQEKNLLDIDAPVTVCGDIHGQFFDLMKLFEVGGSPANTRYLFLGDYVDRGYFSIECVLYLWAL
KILYPKTLFLLRGNHECRHLTEYFTFKQECKIKYSERVYDACMDAFDCLPLAALMNQQFLCVHGGLSP
EINTLDDIRKLDRFKEPPAYGPMCDILWSDPLEDFGNEKTQEHFTHNTVRGCSYFYSYPAVCDFLQHN
NLLSILRAHEAQDAGYRMYRKSQTTGFPSLITIFSAPNYLDVYNNKAAVLKYENNVMNIRQFNCSPHP
YWLPNFMDVFTWSLPFVGEKVTEMLVNVLNICSDDELGSEEDGFDGATAAARKEVIRNKIRAIGKMAR
VFSVLREESESVLTLKGLTPTGMLPSGVLSGGKQTLQSAIKGFSPQHKITSFEEAKGLDRINERMPPR
RDAMPSDANLNSINKALASETNGTDSNGSNSSNIQ

| | |
|---|---|
| SEQ ID NO:21 | human PTPN3a nucleic acid sequence |

| | | |
|---|---|---|
| HUM225350 | accession:M64572 | coding sequence:24 . . . 2765 |

CTGCAGGTTATTCAGCGATAGTTATGACCTCCCGGTTACGTGCGTTGGGTGGAAGAATTAATAATATA
CGCACCTCGGAGTTACCCAAAGAGAAAACTCGATCAGAAGTCATTTGCAGCATCCACTTTTTAGATGG
CGTGGTACAGACCTTTAAAGTTACTAAACAAGACACTGGCCAGGTTCTTCTGGATATGGTGCACAACC
ACCTGGGTGTGACTGAAAAGGAATATTTTGGTTTACAGCATGATGACGACTCCGTGGACTCTCCTAGA
TGGCTGGAAGCAAGCAAACCCATCAGGAAGCAGTTAAAAGGAGGTTTCCCCTGTACCCTGCATTTTCG
AGTAAGATTTTTTATACCTGATCCCAACACACTGCAGCAAGAACAAACCAGGCACTTGTATTTCTTAC
AACTGAAGATGGATATTTGCGAAGGAAGGTTAACCTGCCCTCTTAACTCAGCAGTGGTTCTAGCGTCC
TATGCCGTACAATCTCATTTTGGAGACTATAATTCTTCCATACATCATCCAGGCTATCTTTCCGATAG
TCACTTTATACCCGATCAAAATGAGGACTTTTTAACAAAAGTCGAATCTCTGCATGAGCAGCACAGTG
GGCTAAAACAATCAGAAGCAGAATCCTGCTATATCAACATAGCGCGGACCCTCGACTTCTATGGAGTA
GAACTGCACAGTGGTAGGGATCTGCACAATTTAGACCTAATGATTGGAATTGCTTCCGCGGGTGTTGC
TGTGTACCGAAAATACATTTGCACAAGTTTCTATCCTTGGGTGAACATTCTCAAAATTTCTTTCAAAA
GGAAAAAGTTCTTCATACATCAGCGACAGAAACAGGCTGAATCCAGGGAACATATTGTGGCCTTCAAC
ATGCTGAATTACCGATCTTGCAAAAACTTGTGGAAATCCTGTGTTGAGCACCATACGTTCTTTCAGGC
AAAGAAGCTACTACCTCAGGAAAAGAATGTTCTGTCTCAGTACTGGACTATGGGCTCTCGGAACACCA
AAAAGTCGGTAAATAACCAATATTGCAAAAAGGTGATTGGCGGGATGGTGTGGAACCCAGCCATGCGG
AGATCCTTATCAGTGGAGCACTTAGAAACCAAGAGTCTGCCTTCTCGTTCCCCTCCCATTACTCCCAA
CTGGCGAAGTCCTCGGCTCCGGCACGAAATCCGAAAGCCACGCCACTCTTCTGCAGATAACCTTGCAA
ATGAAATGACCTACATCACGGAAACGGAAGATGTATTTTACACGTACAAGGGCTCTCTGGCCCCTCAA
GACAGCGATTCTGAAGTTTCTCAGAACCGAAGCCCGCACCAAGAGAGTTTATCCGAGAACAATCCGGC
ACAAAGCTACCTGACCCAGAAGTCATCCAGTTCTGTGTCTCCATCTTCAAATGCTCCAGGCTCCTGCT
CACCTGACGGCGTTGATCAGCAGCTCTTAGATGACTTCCACAGGGTGACCAAAGGGGGCTCCACCGAG
GACGCCAGCCAGTACTACTGTGACAAGAATGATAATGGTGACAGCTACTTAGTCTTGATCCGTATCAC
ACCAGATGAAGATGGAAAATTTGGATTTAATCTTAAGGGAGGAGTGGATCAAAAGATGCCTCTTGTGG
TATCAAGGATAAACCCAGAGTCACCTGCGGACACCTGCATTCCTAAGCTGAACGAAGGGGATCAAATC
GTGTTAATCAATGGCCGGGACATCTCAGAACACACGCATGACCAAGTGGTGATGTTCATCAAAGCCAG
CCGGGAGTCCCACTCACGGGAGCTGGCCCTGGTGATCAGGAGGAGAGCTGTCCGCTCATTTGCTGACT
TCAAGTCTGAAGATGAACTGAACCAGCTTTTCCCCGAAGCCATTTTCCCCATGTGTCCGGAGGGTGGG
GACACTTTGGAGGGATCCATGGCACAGCTAAAGAAGGGCCTCGAAAGCGGGACGGTGCTGATCCAGTT
TGAGCAACTCTACAGAAAAAAGCCAGGTTTGGCCATCACGTTTGCAAAGCTGCCTCAAAATTTGGACA
AAAACCGATATAAAGATGTGCTGCCTTATGACACCACCCGGGTATTATTGCAGGGAAATGAAGATTAT
ATTAATGCAAGTTACGTGAACATGGAAATTCCTGCTGCTAACCTTGTGAACAAGTACATCGCCACTCA
GGGGCCCCTGCCGCATACCTGTGCACAGTTTTGGCAGGTTGTCTGGGATCAGAAGTTGTCACTCATTG
TCATGTTGACGACTCTCACAGAACGAGGGCGGACCAAATGTCACCAGTACTGGCCAGATCCCCCCGAC
GTCATGAACCACGGCGGCTTTCACATCCAGTGTCAGTCAGAGGACTGCACCATCGCCTATGTGTCCCG
AGAAATGCTGGTCACAAACACCCAGACCGGGGAAGAACACACAGTGACACATCTCCAGTACGTCGCAT
GGCCTGACCACGGTATACCCGATGACTCCTCCGACTTTCTGGAATTTGTAAACTATGTGAGGTCTCTG
AGAGTGGACAGCGAGCCTGTCCTAGTTCACTGCAGTGCTGGAATAGGTCGAACCGGTGTGTTGGTCAC
TATGGAAACAGCCATGTGCCTAACTGAGAGGAACCTGCCCATTTACCCACTGGATATTGTCCGAAAAA
TGCGAGACCAGCGCGCCATGATGGTGCAGACATCAAGCCAGTACAAGTTTGTGTGTGAAGCGATTCTT
CGTGTGTATGAAGAAGGTTTAGTCCAAATGCTGGATCCTAGTTAAGACAACTGTGAAAAAGTTCATTC
CTCTTTCCCAAGGGCATCCTCCTTGAAAGAGGAGGACAGACCTCTCTGGAAGCAGCAAGAGGAACCAG
TAGCTGTGGGAAAGGAATGGGCACCTCTGAACCCAGGCACTTTAAACTTCTATAGAAAAGATATCGTG
TACATAGGAACTGGTGTAGATAAGCATGCAATTATGGCATCATTTAGGCCTGTATTTCTATGGAAAGA
TACAAAAAGGATCTCAGTTTGGGGCCTGTCCTAATGCCTTCTTCCCTAACATCACCACACACACCCCT
GTCGGCATCCTGGAGCAATTGAGACCGGACACCCACAGAGCTGTTGTCCTCCCAGCAACAAGATGGTG
TGGTTATCTTGGGTCATTTGGATGTTTTGTTTGTTTCTGTGTGTCAGACTGTAAGGGCTGAGCTTTCT
GTGCTTCTAGGTGGAGCTGGAACAATTCAGATTCACCCGCCCTGATGCTAAGGAAACCCTGACGTATG
TACTAGATGGCAGGGCACTGGGGGTCAGGCTGAAGGCTGAGCAACACCTCTCTGCCCTCCCTCCCTTT
GTCCCATCTCCCAGCGACTTCCAATATTCATGTTTCTGAGAATTGTGTCCCTCTTCAGTTCCCTCTTG
GTGCCTAACCTGGATTAGTAATGTGCATTCAGGTGAATTTTCAGCTGAGGCTCTGAGAACTGGTACTC
TCAGTGTGTTCTGGTCATCTTGTGGCTTAGTTGTAGAAGCAGGTGTGTCTCTTGCCTCTGCTTGCCTC
CTACTGCACACTCAGCACCCAGGACTGGAATCACCGACTACTGAATCTCCTACATGTATTGCTGCTAC
TTCAAGCTCCTCCACTTGAAACCTTATGATTTTCCAAGGGGAGATGGGACAGTGTCATCTAAATATTC
CGAATGTTTGGCCTTCTGAGAAAAGAGCTTCTAGTAATTGAACCATGGGTTTCCCAGCTTCTGGAGGG
TTGGCCGTGGGCTGTGTACATGTGTGTGCCCAGGGGTGAGTGTTTCTCAGGATTCCTAACGATTCAAA
TTACCGTTGAGTATATATAAAGAATCGAGTCTCTGTATGGAAGAACAAATGTGTGCATTCACCCCCAG
TCACAATGGTCTCCATTGCATTTCAAAGGAGAGGATCAGACTATCTGAATATAAACACAATCTGATGT
TAATTTATTCTAAGAACACCATCATTTTGATTGTCCTAAA

| | |
|---|---|
| SEQ ID NO:22 | human PTPN3a polypeptide sequence |

| | |
|---|---|
| Protein sequence | protein_id:gi179913 |

MTSRLRALGGRINNIRTSELPKEKTRSEVICSIHFLDGVVQTFKVTKQDTGQVLLDMVHNHLGVTEKE
YFGLQHDDDSVDSPRWLEASKPIRKQLKGGFPCTLHFRVRFFIPDPNTLQQEQTRHLYFLQLKMDICE
GRLTCPLNSAVVLASYAVQSHFGDYNSSIHHPGYLSDSHFIPDQNEDFLTKVESLHEQHSGLKQSEAE
SCYINIARTLDFYGVELHSGRDLHNLDLMIGIASAGVAVYRKYICTSFYPWVNILKISFKRKKFFIHQ
RQKQAESREHIVAFNMLNYRSCKNLWKSCVEHHTFFQAKKLLPQEKNVLSQYWTMGSRNTKKSVNNQY
CKKVIGGMVWNPAMRRSLSVEHLETKSLPSRSPPITPNWRSPRLRHEIRKPRHSSADNLANEMTYITE
TEDVFYTYKGSLAPQDSDSEVSQNRSPHQESLSENNPAQSYLTQKSSSSVSPSSNAPGSCSPDGVDQQ
LLDDFHRVTKGGSTEDASQYYCDKNDNGDSYLVLIRITPDEDGKFGFNLKGGVDQKMPLVVSRINPES
PADTCIPKLNEGDQIVLINGRDISEHTHDQVVMFIKASRESHSRELALVIRRRAVRSFADFKSEDELN
QLFPEAIFPMCPEGGDTLEGSMAQLKKGLESGTVLIQFEQLYRKKPGLAITFAKLPQNLDKNRYKDVL
PYDTTRVLLQGNEDYINASYVNMEIPAANLVNKYIATQGPLPHTCAQFWQVVWDQKLSLIVMLTTLTE
RGRTKCHQYWPDPPDVMNHGGFHIQCQSEDCTIAYVSREMLVTNTQTGEEHTVTHLQYVAWPDHGIPD
DSSDFLEFVNYVRSLRVDSEPVLVHCSAGIGRTGVLVTMETAMCLTERNLPIYPLDIVRKMRDQRAMM
VQTSSQYKFVCEAILRVYEEGLVQMLDPS

| | |
|---|---|
| SEQ ID NO:23 | mouse PTPN3 nucleic acid sequence |

| | |
|---|---|
| accession:XM_143789 | coding sequence:1 . . . 3087 |

ATGGTGAATAAGCCAAGGGACCCCCCCCCCCAACCTGGAATCCAGGGTATCTTTCATCCAGCTCAGGA
AACACCAACCGGAATTCCTGGGCTCCAGAGAACTGTAGGATGGGAATATGAAGTTAAGCAGCTGTTTT
CTGGGAAGCTGGCCAGAAAGTACATCCCTGATTCCAGTGACATTTTTATTGAAAAGGGCATGGTGGAG
GTGGTGGGTTACTCGGCGGCCGTTATGACCTCCCGGTTACGTGCGTTGGGTGGAAGAATTAACAATAC
CCGAACCTCTGAATTGCCCAAAGAGAAAACTCGATCCGAGGTCATCTGCAGCATACGGTTTTTAGATG
GCTTGGTACAGACCTTTAAAGTTAACAAACAAGATTTGGGCCAGTCGCTTCTGGATATGGCCTATGGC
CACCTGGGTGTGACGGAGAAGGAATACTTCGGCTTGCAACATGGCGACGACCCAGTGGACTCTCCTAG
ATGGCTTGAGGCAAGCAAACCCCTCAGGAAGCAGCTGAAAGGTGAGTATGCTCTTGCTTCACTTGGTC
GCTGGGTGTACGGCAAACTGTCTTCTCCTTATGGGGGACTTAAGGTGGCGGGAAAACCTAATCTGTTT
TTGAAAAATGTTGTAGGAGGTTTTCCCTGTACCCTGCATTTTCGAGTAAGATATTTTATACCTGATCC
CAACACACTGCAGCAGGAGCAAACCAGGCATCTATATTTCTTACAACTGAAGATGGATGTTTGCGAAG
GAAGGTTAACCTGCCCTCTCAACTCAGCGGTGGTTCTAGCCTCGTACGCAGTACAATCTCATTTTGGA
GACTTTAATTCTTCAATACATCATCCAGGCTATCTTGCCGACAGCCAGTTCATACCAGATCAAAATGA
TGACTTTTTAAGCAAGGTGGAGTCACTCCATGAACAGCACAGTGGGCTAAAGCAGTCGGAAGCCGAAT
CTTGCTACATCAACATAGCTCGAACCCTTGACTTCTACGGAGTGGAGCTTCATGGAGGCAGAGATCTG
CACAACCTAGATCTAATGATCGGGATTGCGTCTGCAGGCATTGCAGTCTACCGAAAATACATTTGCAC
AAGTTTCTACCCTTGGAAAAAGTTCTTCATACACCAGCGGCAAAAACAGGAAGAAAAGATTGTGGCCG
TCAGGTCTTCAGACCCTGTGGCCATCTCTGCTGAATCCAGGGAACATATCGTGGCCTTCAACATGCTG
AATTACCGATCTTGCAAAAACTTGTGGAAATCCTGCGTCGAACACCATTCGTTCTTTCAGGCAAAAAA
GTTACTACCTCAGGAAAAGAATGTTCTCTCTCAGTACTGGACTCTAGGCTCCAGGAACCCCAAAAAGT
CTGTAAATAACCAATATTGCAAAAAGGTGATTGGAGGGATGGTGTGGAACCCAGTCATGAGGAGGTCC
TTATCAGTGGAACGGCTGGAAACCAAGAGCCTGCCTTCCCGGTCCCCTCCCATCACCCCCAACTGGCG
AAGTCCTCGGCTCCGGCATGAAATCCGCAAGCCCCGCCATTCCTCCGCGGACAACCTGGCCAATGAGA
TGACCTACATCACAGAAACCGAGGATGTGTTCTATACCTACAAGGGACCCCTGTCCCCCAAAGACAGC
GACTCTGAAGTCTCCCAGAACCACAGCCCACACCGGGAGAGTTTGTCCGAAAACAACCCAGCCCAAAG
CTGCCTGACCCAGAAGTCATCCAGTTCTGTGTCTCCATCTTCGAATGCTCCAGGCTCCTGCTCTCCAG
ATGGAGTTGACCAGCGGTTCTTAGAAGACTACCACAAGGTGACCAAAGGGGGGTTCGTCGAGGACGCC
AGCCAGTACTACTGTGATAAGAGTGACGATGGAGATGGCTACCTAGTCCTGATCCGGATCACACCAGA
TGAAGAGGGAAGATTTGGCTTTAACCTTAAGGCAGATACCTGCATGCCTAAGCTGAATGAGGGGGATC
AGATCGTGTTGATCAATGGCCGGGACATCTCAGAGCACACTCATGACCAGGTGGTGATGTTCATCAAA
GCAAGCCGGGAGTCCCACTCGAGAGAACTGGCCCTGGTGATCAGGAGAAAAGGAAAAGCTACCTTTGT
GGGCCATGAGGGACTGGTACCTGCTAGAGCTGTGCGCTCGCTGGCGGAGATCAGATCAGAAGATGAGC
TGAGTCAGCTTTTCCCAGAAGCCATGTTTCCTGCATGTCCTGAGGGTGGGGACAGTCTGGAGGGATCC
ATGGAGCTGCTGAAGAAAGGTCTGGAAAGCGGTACAGTGTTGATCCAGTTTGAGCAACTCTACAGAAA
GAAGCCCGGCTTAGCCGTCTCATTTGCAAAGCTGCCTCAGAATTTGGATAAAAACCGATACAAAGATG
TGTTGCCTTATGATACCACGCGGGTATTATTGCAGGGAAATGAAGATTACATTAATGCCAGTTATGTG
AACATGGAGATGCCCGCTGCTAACCTTGTGAACAAGTACATTGCCACTCAGGGACCGCTGCCAAACAC
CTGTGCACAGTTTTGGCAAGTTGTCTGGGATCAGAAGTTGTCTCTTGTTGTCATGCTGACGACTCTCA
CCGAGCGAGGGCGGACCAAATGTCACCAGTACTGGCCGGATCCCCCTGACATCATGGACCATGGCATC
TTTCACATCCAGTGTCAGACAGAGGACTGTACCATTGCTTACGTGTCCCGAGAAATGCTAGTTACAAA
CACGGAGACCGGGGAAGAACACACAGTGACCCATCTCCAGTATGTGGCGTGGCCTGACCACGGCGTAC
CTGATGACTCATCAGACTTCCTGGAGTTTGTGAAGTATGTGAGGTCCCTGAGGGTCGACGGTGAGCCT
GCCCTCGTTCACTGCAGTGCTGGAATAGGTCGGACAGGTGTTTTAGTCACTATGGAAACAGCCATGTG
CTTAATTGAGAGGAACCTGCCTGTTTACCCACTGGATATTGTCCGGAAAATGAGAGACCAGCGCGCGA
TGATGGTGCAGACGTCAAGCCAATACAAGTTTGTGTGTGAAGCGATCCTTCGAGTGTACGAAGAAGGC
TTAGTCCAGAGGCTGGATCCCAGTTAG

| | |
|---|---|
| SEQ ID NO:24 | mouse PTPN3 polypeptide sequence |

| | |
|---|---|
| Protein sequence | accession:gi20828736 |

MVNKPRDPPPQPGIQGIFHPAQETPTGIPGLQRTVGWEYEVKQLFSGKLARKYIPDSSDIFIEKGMVE
VVGYSAAVMTSRLRALGGRINNTRTSELPKEKTRSEVICSIRFLDGLVQTFKVNKQDLGQSLLDMAYG
HLGVTEKEYFGLQHGDDPVDSPRWLEASKPLRKQLKGEYALASLGRWVYGKLSSPYGGLKVAGKPNLF
LKNVVGGFPCTLHFRVRYFIPDPNTLQQEQTRHLYFLQLKMDVCEGRLTCPLNSAVVLASYAVQSHFG
DFNSSIHHPGYLADSQFIPDQNDDFLSKVESLHEQHSGLKQSEAESCYINIARTLDFYGVELHGGRDL
HNLDLMIGIASAGIAVYRKYICTSFYPWKKFFIHQRQKQEEKIVAVRSSDPVAISAESREHIVAFNML
NYRSCKNLWKSCVEHHSFFQAKKLLPQEKNVLSQYWTLGSRNPKKSVNNQYCKKVIGGMVWNPVMRRS
LSVERLETKSLPSRSPPITPNWRSPRLRHEIRKPRHSSADNLANEMTYITETEDVFYTYKGPLSPKDS
DSEVSQNHSPHRESLSENNPAQSCLTQKSSSSVSPSSNAPGSCSPDGVDQRFLEDYHKVTKGGFVEDA
SQYYCDKSDDGDGYLVLIRITPDEEGRFGFNLKADTCMPKLNEGDQIVLINGRDISEHTHDQVVMFIK
ASRESHSRELALVIRRKGKATFVGHEGLVPARAVRSLAEIRSEDELSQLFPEAMFPACPEGGDSLEGS
MELLKKGLESGTVLIQFEQLYRKKPGLAVSFAKLPQNLDKNRYKDVLPYDTTRVLLQGNEDYINASYV
NMEMPAANLVNKYIATQGPLPNTCAQFWQVVWDQKLSLVVMLTTLTERGRTKCHQYWPDPPDIMDHGI
FHIQCQTEDCTIAYVSREMLVTNTETGEEHTVTHLQYVAWPDHGVPDDSSDFLEFVKYVRSLRVDGEP
ALVHCSAGIGRTGVLVTMETAMCLIERNLPVYPLDIVRKMRDQRAMMVQTSSQYKFVCEAILRVYEEG
LVQRLDPS

| | |
|---|---|
| SEQ ID NO:25 | human PTPN3b splice variant nucleic acid sequence |

| | |
|---|---|
| Nucleotide sequence | coding sequence:1 . . . 2607 |

ATGACCTCCCGGTTACGTGCGTTGGGTGGAAGAATTAATAATATACGCACCTCGGAGTTACCCAAAGA
GAAAACTCGATCAGAAGTCATTTGCAGCATCCACTTTTTAGATGGCGTGGTACAGACCTTTAAAGTTA
CTAAACAAGACACTGGCCAGGTTCTTCTGGATATGGTGCACAACCACCTGGGTGTGACTGAAAAGGAA
TATTTTGGTTTACAGCATGATGACGACTCCGTGGACTCTCCTAGATGGCTGGAAGCAAGCAAACCCAT
CAGGAAGCAGTTAAAAGGAGGTTTCCCCTGTACCCTGCATTTTCGAGTAAGATTTTTTATACCTGATC
CCAACACACTGCAGCAAGAACAAACCAGGCACTTGTATTTCTTACAACTGAAGATGGATATTTGCGAA
GGAAGGTTAACCTGCCCTCTTAACTCAGCAGTGGTTCTAGCGTCCTATGCCGTACAATCTCATTTTGG
AGACTATAATTCTTCCATACATCATCCAGGCTATCTTTCCGATAGTCACTTTATACCCGATCAAAATG
AGGACTTTTTAACAAAAGTCGAATCTCTGCATGAGCAGCACAGTGGGCTAAAACAATCAGAAGCAGAA
TCCTGCTATATCAACATAGCGCGGACCCTCGACTTCTATGGAGTAGAACTGCACAGTGGTAGGGATCT
GCACAATTTAGACCTAATGATTGGAATTGCTTCCGCGGGTGTTGCTGTGTACCGAAAATACATTTGCA
CAAGTTTCTATCCTTGGGTGAACATTCTCAAAATTTCTTTCAAAAGGAAAAAGTTCTTCATACATCAG
CGACAGAAACAGGCTGAATCCAGGGAACATATTGTGGCCTTCAACATGCTGAATTACCGATCTTGCAA
AAACTTGTGGAAATCCTGTGTTGAGCACCATACGTTCTTTCAGGCAAAGAAGCTACTACCTCAGGAAA
AGAATGTTCTGTCTCAGTACTGGACTATGGGCTCTCGGAACACCAAAAAGCGAAGTCCTCGGCTCCGG
CACGAAATCCGAAAGCCACGCCACTCTTCTGCAGATAACCTTGCAAATGAAATGACCTACATCACGGA
AACGGAAGATGTATTTTACACGTACAAGGGCTCTCTGGCCCCTCAAGACAGCGATTCTGAAGTTTCTC
AGAACCGAAGCCCGCACCAAGAGAGTTTATCCGAGAACAATCCGGCACAAAGCTACCTGACCCAGAAG
TCATCCAGTTCTGTGTCTCCATCTTCAAATGCTCCAGGCTCCTGCTCACCTGACGGCGTTGATCAGCA
GCTCTTAGATGACTTCCACAGGGTGACCAAAGGGGGCTCCACCGAGGACGCCAGCCAGTACTACTGTG
ACAAGAATGATAATGGTGACAGCTACTTAGTCTTGATCCGTATCACACCAGATGAAGATGGAAAATTT
GGATTTAATCTTAAGGGAGGAGTGGATCAAAAGATGCCTCTTGTGGTATCAAGGATAAACCCAGAGTC
ACCTGCGGACACCTGCATTCCTAAGCTGAACGAAGGGGATCAAATCGTGTTAATCAATGGCCGGGACA
TCTCAGAACACACGCATGACCAAGTGGTGATGTTCATCAAAGCCAGCCGGGAGTCCCACTCACGGGAG
CTGGCCCTGGTGATCAGGAGGAGAGCTGTCCGCTCATTTGCTGACTTCAAGTCTGAAGATGAACTGAA
CCAGCTTTTCCCCGAAGCCATTTTCCCCATGTGTCCGGAGGGTGGGGACACTTTGGAGGGATCCATGG
CACAGCTAAAGAAGGGCCTCGAAAGCGGGACGGTGCTGATCCAGTTTGAGCAACTCTACAGAAAAAAG
CCAGGTTTGGCCATCACGTTTGCAAAGCTGCCTCAAAATTTGGACAAAAACCGATATAAAGATGTGCT
GCCTTATGACACCACCCGGGTATTATTGCAGGGAAATGAAGATTATATTAATGCAAGTTACGTGAACA
TGGAAATTCCTGCTGCTAACCTTGTGAACAAGTACATCGCCACTCAGGGGCCCCTGCCGCATACCTGT
GCACAGTTTTGGCAGGTTGTCTGGGATCAGAAGTTGTCACTCATTGTCATGTTGACGACTCTCACAGA
ACGAGGGCGGACCAAATGTCACCAGTACTGGCCAGATCCCCCCGACGTCATGAACCACGGCGGCTTTC
ACATCCAGTGTCAGTCAGAGGACTGCACCATCGCCTATGTGTCCCGAGAAATGCTGGTCACAAACACC
CAGACCGGGGAAGAACACACAGTGACACATCTCCAGTACGTCGCATGGCCTGACCACGGTATACCCGA
TGACTCCTCCGACTTTCTGGAATTTGTAAACTATGTGAGGTCTCTGAGAGTGGACAGCGAGCCTGTCC
TAGTTCACTGCAGTGCTGGAATAGGTCGAACCGGTGTGTTGGTCACTATGGAAACAGCCATGTGCCTA
ACTGAGAGGAACCTGCCCATTTACCCACTGGATATTGTCCGAAAAATGCGAGACCAGCGCGCCATGAT
GGTGCAGACATCAAGCCAGTACAAGTTTGTGTGTGAAGCGATTCTTCGTGTGTATGAAGAAGGTTTAG
TCCAAATGCTGGATCCTAGTTAA

| | |
|---|---|
| SEQ ID NO:26 | human PTPN3b splice variant polypeptide sequence |

MTSRLRALGGRINNIRTSELPKEKTRSEVICSIHFLDGVVQTFKVTKQDTGQVLLDMVHNHLGVTEKE
YFGLQHDDDSVDSPRWLEASKPIRKQLKGGFPCTLHFRVRFFIPDPNTLQQEQTRHLYFLQLKMDICE
GRLTCPLNSAVVLASYAVQSHFGDYNSSIHHPGYLSDSHFIPDQNEDFLTKVESLHEQHSGLKQSEAE
SCYINIARTLDFYGVELHSGRDLHNLDLMIGIASAGVAVYRKYICTSFYPWVNILKISFKRKKFFIHQ
RQKQAESREHIVAFNMLNYRSCKNLWKSCVEHHTFFQAKKLLPQEKNVLSQYWTMGSRNTKKRSPRLR
HEIRKPRHSSADNLANEMTYITETEDVFYTYKGSLAPQDSDSEVSQNRSPHQESLSENNPAQSYLTQK
SSSSVSPSSNAPGSCSPDGVDQQLLDDFHRVTKGGSTEDASQYYCDKNDNGDSYLVLIRITPDEDGKF
GFNLKGGVDQKMPLVVSRINPESPADTCIPKLNEGDQIVLINGRDISEHTHDQVVMFIKASRESHSRE
LALVIRRRAVRSFADPKSEDELNQLFPEAIFPMCPEGGDTLEGSMAQLKKGLESGTVLIQFEQLYRKK
PGLAITFAKLPQNLDKNRYKDVLPYDTTRVLLQGNEDYINASYVNMEIPAANLVNKYIATQGPLPHTC
AQFWQVVWDQKLSLIVMLTTLTERGRTKCHQYWPDPPDVMNHGGFHIQCQSEDCTIAYVSREMLVTNT
QTGEEHTVTHLQYVAWPDHGIPDDSSDFLEFVNYVRSLRVDSEPVLVHCSAGIGRTGVLVTMETAMCL
TERNLPIYPLDIVRKMRDQRAMMVQTSSQYKFVCEAILRVYEEGLVQMLDPS

| | |
|---|---|
| SEQ ID NO:27 | human DUSP3 nucleic acid sequence |

| | | |
|---|---|---|
| HUM230054 | accession:L05147 | CDS:29 . . . 586 |

GCCGGGCGTGCAGGGCCCCGCCGCCGCCATGTCGGGCTCGTTCGAGCTCTCGGTGCAGGATCTCAACG
ACCTGCTCTCGGACGGCAGCGGCTGCTACAGCCTCCCGAGCCAGCCCTGCAACGAGGTCACCCCGCGG
ATCTACGTGGGCAACGCGTCTGTGGCTCAGGACATCCCCAAGCTGCAGAAACTAGGCATCACCCATGT
GCTGAACGCGGCTGAGGGCAGGTCCTTCATGCACGTCAACACCAATGCCAACTTCTACAAGGACTCCG
GCATCACATACCTGGGCATCAAGGCCAACGACACACAGGAGTTCAACCTCAGCGCTTACTTTGAAAGG
GCTGCCGACTTCATTGACCAGGCTTTGGCTCAAAAGAATGGCCGGGTGCTCGTCCACTGCCGGGAAGG
TTATAGCCGCTCCCCAACGCTAGTTATCGCCTACCTCATGATGCGGCAGAAGATGGACGTCAAGTCTG
CCCTGAGCATCGTGAGGCAGAACCGTGAGATCGGCCCCAACGATGGCTTCCTGGCCCAGCTCTGCCAG
CTCAATGACAGACTAGCCAAGGAGGGGAAGTTGAAACCCTAGGGCACCCCCACCGCCTCTGCTCGAGA
GGTCCGTGGGGGAGGCCGTGGGAAAGGTGTCCGAGCTGCCATGTTTAGGAAACACACTGTACCCTGCT
CCCAGCATCACAAGGCACTTGTCTACAAGTGTGTCCCAACACAGTCCTGGGCCACTTTCCCCACCCTG
GGGAGCACATAAAGAAGCTTGCCAAGGGGGGCGTCCTTGCTCCCCAGTTGTCCTGTTTCTGTAACTTA
TGATGTCTTTTCCCTGAGATGGGGGCTCAGAGGGGGAAGGCCTGT

| | |
|---|---|
| SEQ ID NO:28 | human DUSP3 polypeptide sequence |

protein_id:gi181840
MSGSFELSVQDLNDLLSDGSGCYSLPSQPCNEVTPRIYVGNASVAQDIPKLQKLGITHVLNAAEGRSF
MHVNTNANFYKDSGITYLGIKANDTQEFNLSAYFERAADFIDQALAQKNGRVLVHCREGYSRSPTLVI
AYLMMRQKMDVKSALSIVRQNREIGPNDGFLAQLCQLNDRLAKEGKLKP

| | |
|---|---|
| SEQ ID NO:29 | mouse DUSP3 nucleotide sequence |

accession:XM_109743
CCGCTGACCCGGCTTCTCCCCCTCGTTCGCTCCTGCCCTGGCGTGCAGCGCCCCGCCGCCGCCATGTC
CAGCTCGTTCGAACTCTCGGTGCAAGATCTCAACGACCTGCTCTCGGATGGCAGCGGCTGCTACAGCC
TGCCGAGCCAGCCCTGCAACGAGGTCGTCCCGAGGGTCTACGTGGGCAACGCGTCTGTGGCTCAGGAC
ATCACCCAGCTGCAGAAACTGGGCATCACCCACGTCCTGAATGCTGCCGAGGGCAGGTCCTTCATGCA
CGTCAACACCAGTGCTAGCTTCTACGAGGATTCTGGCATCACCTACTTGGGCATCAAGGCCAATGATA
CGCAGGAGTTCAACCTCAGTGCTTACTTTGAAAGGGCCACAGATTTCATTGACCAGGCGCTGGCCCAT
AAAAATGGCCGGGTGCTTGTCCATTGCCGCGAGGGCTACAGCCGCTCCCCAACGCTAGTTATCGCCTA
CCTCATGATGCGGCAGAAGATGGACGTCAAGTCTGCTCTGAGTACTGTGAGGCAGAATCGTGAGATCG
GCCCCAACGATGGCTTCCTGGCCCAACTCTGCCAGCTCAATGACAGACTAGCCAAGGAGGGCAAGGTG
AAACTCTAGGGTGCCCACAGCCTCTTTTGCAGAGGTCTGACTGGGAGGGCCCTGGCAGCCATGTTTAG
GAAACACAGTATACCCACTCCCTGCACCACCAGACACGTGCCCACATCTGTCCCACTCTGGTCCTCGG
GGGCCACTCCACCCTTAGGGAGCACATGAAGAAGCTCCCTAAGAAGTTCTGCTCCTTAGCCATCCTTT
CCTGTAATTTATGTCTCTCCCTGAGGTGAGGTTCAGGTTTATGTCCCTGTCTGTGGCATAGATACATC
TCAGTGACCCAGGGTGGGAGGGCTATCAGGGTGCATGGCCCGGGACACGGGCACTCTTCATGACCCCT
CCCCCACCTGGGTTCTTCCTGTGTGGTCCAGAACCACGAGCCTGGTAAAGGAACTATGCAAACACAGG
CCCTGACCTCCCCATGTCTGTTCCTGGTCCTCACAGCCCGACACGCCCTGCTGAGGCAGACGAATGAC
ATTAAGTTCTGAAGCAGAGTGGAGATAGATTAGTGACTAGATTTCCAAAAAGAAGGAAAAAAAAGGCT
GCATTTTAAAATTATTTCCTTAGAATTAAAGATACTACAT

| | |
|---|---|
| SEQ ID NO:30 | mouse DUSP3 polypeptide sequence |

accession:gi20344845
MSSSFELSVQDLNDLLSDGSGCYSLPSQPCNEVVPRVYVGNASVAQDITQLQKLGITHVLNAAEGRSF
MHVNTSASFYEDSGITYLGIKANDTQEFNLSAYFERATDFIDQALAHKNGRVLVHCREGYSRSPTLVI
AYLMMRQKMDVKSALSTVRQNREIGPNDGFLAQLCQLNDRLAKEGKVKL

| | |
|---|---|
| SEQ ID NO:31 | human RGS10 nucleic acid sequence |

| | | |
|---|---|---|
| HUM241658 | accession:AF045229 | coding sequence:133 . . . 636 |

TACCGAGCTCGGATCCACTAGTAACGGCCGCCAGTGTGCTGGAATTCGCCCTTACTCACTATAGGGCT
CGAGCGGCCGCCCGGGCAGGTGGATTGTTGGTCTGCGTGGAACTTCTCAGGTGGACACCAGAGCATGG
AACACATCCACGACAGCGATGGCAGTTCCAGCAGCAGCCACCAGAGCCTCAAGAGCACAGCCAAATGG
GCGGCATCCCTGGAGAATCTGCTGGAAGACCCAGAAGGCGTGAAAAGATTTAGGGAATTTTTAAAAAA
GGAATTCAGTGAAGAAAATGTTTTGTTTTGGCTAGCATGTGAAGATTTTAAGAAAATGCAAGATAAGA
CGCAGATGCAGGAAAAGGCAAAGGAGATCTACATGACCTTTCTGTCCAGCAAGGCCTCATCACAGGTC
AACGTGGAGGGGCAGTCTCGGCTCAACGAGAAGATCCTGGAAGAACCGCACCCTCTGATGTTCCAGAA
ACTCCAGGACCAGATCTTTAATCTCATGAAGTACGACAGCTACAGCCGCTTTCTTAAGTCTGACTTGT
TTTTAAAACACAAGCGAACCGAGGAAGAGGAAGAAGATTTGCCTGATGCTCAAACTGCAGCTAAAAGA
GCTTCCAGAATTTATAACACATGAGCCCCCAAAAAGCCGGGACTGGCAGCTTTAAGAAGCAAAGGAAT
TTCCTCTCAGGACGTGCCGGGTTTATCATTGCTTTGTTATTTGTAAGGACTGAAATGTACAAAACCCT
TCAAT

| | |
|---|---|
| SEQ ID NO:32 | human RGS10 polypeptide sequence |

protein_id:gi2906030
MEHIHDSDGSSSSSHQSLKSTAKWAASLENLLEDPEGVKRFREFLKKEFSEENVLFWLACEDFKKMQD
KTQMQEKAKEIYMTFLSSKASSQVNVEGQSRLNEKILEEPHPLMFQKLQDQIFNLMKYDSYSRFLKSD
LFLKHKRTEEEEEDLPDAQTAAKRASRIYNT

| | |
|---|---|
| SEQ ID NO:33 | mouse RGS10 nucleic acid sequence |

| | |
|---|---|
| NM_026418 | coding sequence:60 . . . 605 |

GCTCTTCGGGCTTAGCCGCCGCGCTGCCCGGCTGCTCCGTCCTCTGGACGCCCGCGGCGATGTTCACC
CGCGCCGTGAGCCGACTGAGCAGGAAGCGGCCGCCGTCTGATATCCATGACGGAGATGGGAGCTCAAG
CAGCGGCCACCAGAGCCTTAAGAGCACAGCCAAGTGGGCATCCTCCCTGGAGAATCTTCTGGAAGACC
CAGAAGGGGTGCAGAGATTCAGGGAGTTTCTGAAGAAGGAATTCAGCGAAGAGAATGTCTTGTTTTGG
CTAGCGTGTGAAGATTTCAAGAAAACGGAGGACAGGAAGCAGATGCAGGAAAAGGCCAAGGAGATCTA
CATGACCTTCCTGTCCAATAAGGCCTCTTCACAAGTCAACGTGGAGGGGCAGTCTCGGCTCACTGAAA
AGATTCTGGAAGAGCCACACCCTCTGATGTTCCAAAAGCTCCAGGACCAGATCTTCAATCTCATGAAG
TATGACAGCTACAGCCGCTTCTTGAAGTCTGACTTGTTTCTGAAACCCAAGCGAACTGAGGAAGAGGA
AGAAGAGCCCCCGGATGCTCAGACCGCAGCTAAGCGAGCCTCCAGAATTTACAACACATAAGCTGAGC
CCTTCACCCCAGCGAAGGAGAGGGATGGACTCTTAGGACTGTACAGGCTGTCATTTCTTTGTTGTGTT
TGAGGACTGGAGTGTGCTAGACCTTCCCTCTGGATATGTGTATTTTATTAACTGAACAGCAACCTCTG
CATGATGCTAATCTTCCATTAAAAACAAAAGTAGCTTTAAAGTGTCAGTTCACAAAAACACATGAGAT
TCTGCCAATACTGGACACTCAGCCTTTCAATCCTGATTAAAGTGTTCGTGAAGCTACAAGC

| | |
|---|---|
| SEQ ID NO:34 | mouse RGS10 polypeptide sequence |

accession:gi13385914
MFTRAVSRLSRKRPPSDIHDGDGSSSSGHQSLKSTAKWASSLENLLEDPEGVQRFREFLKKEFSEENV
LFWLACEDFKKTEDRKQMQEKAKEIYMTFLSNKASSQVNVEGQSRLTEKILEEPHPLMFQKLQDQIFN
LMKYDSYSRFLKSDLFLKPKRTEEEEEEPPDAQTAAKRASRIYNT

What is claimed is:

1. A method for identifying an agent for treating a diabetic or pre-diabetic individual, the method comprising the steps of:
(i) contacting an agent to a mixture comprising a polypeptide encoded by a nucleic acid that hybridizes under stringent conditions to a nucleic acid encoding SEQ ID NO:2, SEQ ID NO:8, SEQ ID NO:12, SEQ ID NO:16, SEQ ID NO:22, SEQ ID NO:26, SEQ ID NO:28, or SEQ ID NO:32; and
(ii) selecting an agent that modulates the expression or activity of the polypeptide or that binds to the polypeptide, thereby identifying an agent for treating a diabetic or pre-diabetic individual.

2. The method of claim 1, the method further comprising selecting an agent that modulates insulin sensitivity.

3. The method of claim 1, wherein step (ii) comprises selecting an agent that modulates expression of the polypeptide.

4. The method of claim 1, wherein step (ii) comprises selecting an agent that modulates the activity of the polypeptide.

5. The method of claim 1, wherein step (ii) comprises selecting an agent that specifically binds to the polypeptide.

6. The method of claim 1, wherein the polypeptide is expressed in a cell and the cell is contacted with the agent.

7. The method of claim 1, wherein the polypeptide is SEQ ID NO:2, SEQ ID NO:8, SEQ ID NO:12, SEQ ID NO:16, SEQ ID NO:22, SEQ ID NO:26, SEQ ID NO:28, or SEQ ID NO:32

8. A method of treating a diabetic or pre-diabetic animal, the method comprising administering to the animal a therapeutically effective amount of an agent identified by the method of claim 1.

9. The method of claim 8, wherein the agent is an antibody.

10. The method of claim 9, wherein the antibody is a monoclonal antibody.

11. The method of claim 8, wherein the animal is a human.

12. A method of introducing an expression cassette into a cell, the method comprising,
introducing into the cell an expression cassette comprising a promoter operably linked to a polynucleotide encoding a polypeptide, wherein the polynucleotide hybridizes under stringent conditions to a nucleic acid encoding SEQ ID NO:2, SEQ ID NO:8, SEQ ID NO:12, SEQ ID NO:16, SEQ ID NO:22, SEQ ID NO:26, SEQ ID NO:28, or SEQ ID NO:32.

13. The method of claim 12, wherein the polypeptide comprises SEQ ID NO:2, SEQ ID NO:8, SEQ ID NO:12, SEQ ID NO:16, SEQ ID NO:22, SEQ ID NO:26, SEQ ID NO:28, or SEQ ID NO:32.

14. The method of claim 12, wherein the cell is selected from the group consisting of an adipocyte and a skeletal muscle cell.

15. The method of claim 12, the method further comprising introducing the cell into a human.

16. The method of claim 15, wherein the human is diabetic.

17. The method of claim 15, wherein the human is prediabetic.

18. The method of claim 15, wherein the cell is from the human.

19. A method of diagnosing an individual who has Type 2 diabetes or is prediabetic, the method comprising,
detecting in a sample from the individual the level of a polypeptide or the level of a polynucleotide encoding the polypeptide, wherein the polynucleotide hybridizes under stringent conditions to a nucleic acid encoding an amino acid sequence selected from the group consisting of SEQ ID NO:2, SEQ ID NO:8, SEQ ID NO:12, SEQ ID NO:16, SEQ ID NO:22, SEQ ID NO:26, SEQ ID NO:28, and SEQ ID NO:32;
wherein a modulated level of the polypeptide or polynucleotide in the sample compared to a level of the polypeptide or polynucleotide in either a lean individual or a previous sample from the individual indicates that the individual is diabetic or prediabetic.

20. The method of claim 19, wherein the detecting step comprises contacting the sample with an antibody that specifically binds to the polypeptide.

21. The method of claim 19, wherein the amino acid sequence comprises SEQ ID NO:2, SEQ ID NO:8, SEQ ID NO:12, SEQ ID NO:16, SEQ ID NO:22, SEQ ID NO:26, SEQ ID NO:28, or SEQ ID NO:32

22. The method of claim 19, wherein the detecting step comprises quantifying mRNA encoding the polypeptide.

23. The method of claim 22, wherein the mRNA is reverse transcribed and amplified in a polymerase chain reaction.

24. The method of claim 19, wherein the sample is a blood, urine or tissue sample.

25. An isolated nucleic acid encoding a polypeptide of SEQ ID NO:26.

26. The isolated nucleic acid of claim 25, wherein the polypeptide is encoded by a nucleic acid comprising SEQ ID NO:25.

27. An expression vector comprising the nucleic acid of claim 25.

28. A host cell comprising the expression vector of claim 27.


SEQUENCE LISTING (36 sequences)

SEQ ID NO: 1 - DNA, 1426 bp, Homo sapiens
  human purinergic receptor P2Y, G-protein coupled 1 (P2RY1) cDNA
ccgcctcctacccctcggagccgccgcctaagtcgaggaggagagaatgaccgaggtgctgtggccggctgtccccaacgggacggacgctgccttcctggccggtccgggttcgtcctgggggaacagcacggtcgcctccactgccgccgtctcctcgtcgttcaaatgcgccttgaccaagacgggcttccagttttactacctgccggctgtctacatcttggtattcatcatcggcttcctgggcaacagcgtggccatctggatgttcgtcttccacatgaagccctggagcggcatctccgtgtacatgttcaatttggctctggccgacttcttgtacgtgctgactctgccagccctgatcttctactacttcaataaaacagactggatcttcggggatgccatgtgtaaactgcagaggttcatctttcatgtgaacctctatggcagcatcttgtttctgacatgcatcagtgcccaccggtacagcggtgtggtgtaccccctcaagtccctgggccggctcaaaaagaagaatgcgatctgtatcagcgtgctggtgtggctcattgtggtggtggcgatctcccccatcctcttctactcaggtaccggggtccgcaaaaacaaaaccatcacctgttacgacaccacctcagacgagtacctgcgaagttatttcatctacagcatgtgcacgaccgtggccatgttctgtgtccccttggtgctgattctgggctgttacggattaattgtgagagctttgatttacaaagatctggacaactctcctctgaggagaaaatcgatttacctggtaatcattgtactgactgtttttgctgtgtcttacatccctttccatgtgatgaaaacgatgaacttgagggcccggcttgattttcagaccccagcaatgtgtgctttcaatgacagggtttatgccacgtatcaggtgacaagaggtctagcaagtctcaacagttgtgtggaccccattctctatttcttggcgggagatactttcagaaggagactctcccgagccacaaggaaagcttctagaagaagtgaggcaaatttgcaatccaagagtgaagacatgaccctcaatattttacctgagttcaagcagaatggagatacaagcctgtgaaggcacaagaatctccaaacacctctctgttgtaatatggtaggatgcttaacagaatcaagtacttttcccctctttaactttctagtttagaaaaaaatcaaaccaagaaaatagtgagttaaaaaaataatagaagtagaaatgcccacatccacacttagcttgtttgggtttgctttcacagtctctcttccttctgactagaagtatgtataataaaacaatactacctagttaaaaaaaaaaaaaaaaaaa

SEQ ID NO: 2 - protein, 373 aa, Homo sapiens
  human purinergic receptor P2Y, G-protein coupled 1 (P2RY1)
MTEVLWPAVPNGTDAAFLAGPGSSWGNSTVASTAAVSSSFKCALTKTGFQFYYLPAVYILVFIIGFLGNSVAIWMFVFHMKPWSGISVYMFNLALADFLYVLTLPALIFYYFNKTDWIFGDAMCKLQRFIFHVNLYGSILFLTCISAHRYSGVVYPLKSLGRLKKKNAICISVLVWLIVVVAISPILFYSGTGVRKNKTITCYDTTSDEYLRSYFIYSMCTTVAMFCVPLVLILGCYGLIVRALIYKDLDNSPLRRKSIYLVIIVLTVFAVSYIPFHVMKTMNLRARLDFQTPAMCAFNDRVYATYQVTRGLASLNSCVDPILYFLAGDTFRRRLSRATRKASRRSEANLQSKSEDMTLNILPEFKQNGDTSL

SEQ ID NO: 3 - DNA, 1293 bp, Mus musculus
  mouse purinergic receptor P2Y, G-protein coupled 1 (P2RY1) cDNA
tctagtagctgcctgagttggaaagaagaggatgaccgaggtgccttggtcggttgtccccaacgggacggatgctgcctttctggcgggcctgggctcgctttggggaaacagtactgtcgcctcaactgcagcagtttcctcttcattccaatgtgccctgaccaagaccggtttccagttctactacctgccggctgtctacattttagtgttcatcataggcttcctaggcaacagcgtggctatctggatgttcgttttccacatgaagccttggagcggcatctccgtgtacatgttcaatttggctctggctgactttttgtatgtgctcaccctaccagccctcatcttctactacttcaacaagactgactggatcttcggggatgctatgtgcaagctgcagagattcatcttccacgtaaatctctatggtagcatcttgttcctcacctgcatcagcgcacacaggtacagtggcgtggtgtaccctctcaagtctctgggcaggctcaagaagaagaatgccatttatgtcagcgtgctggtgtggctcattgtggtggtggccatctcccctattctcttctactctggcactgggactcggaaaaacaaaactgtcacctgctatgacaccacgtccaatgattacctgcgaagttatttcatctacagtatgtgcacgactgtggccatgttctgcatccctttggtgctgatcttgggctgttatggattaattgttaaagctttgatttacaatgacctggacaactctccgctccggaggaaatccatttacctggtgataattgtcctgacggtgtttgctgtgtcttatatccctttccatgtgatgaaaacgatgaatttgcgagcacggttggatttccagaccccagaaatgtgtgatttcaacgacagggtttatgccacttatcaggtaacaagaggtctagcaagtctcaacagctgtgtggaccccattctttatttcttggctggagatacattcagaaggagactgtcccgagccaccaggaaagcttccaggaggagtgaggccaatttacaatccaagagtgaagaaatgactctcaatattttgtctgagttcaagcagaatggagacacgagtttgtgaaggcacgagatcctagctcctgagttttgtaacatggtcacaagacatccctgagatgatctatgcatacacaggtcaagaagaagcaacactctattgaccaaaagaccagtgtgtgccacctgagtgagggttttcct

SEQ ID NO: 4 - protein, 373 aa, Mus musculus
  mouse purinergic receptor P2Y, G-protein coupled 1 (P2RY1)
MTEVPWSVVPNGTDAAFLAGLGSLWGNSTVASTAAVSSSFQCALTKTGFQFYYLPAVYILVFIIGFLGNSVAIWMFVFHMKPWSGISVYMFNLALADFLYVLTLPALIFYYFNKTDWIFGDAMCKLQRFIFHVNLYGSILFLTCISAHRYSGVVYPLKSLGRLKKKNAIYVSVLVWLIVVVAISPILFYSGTGTRKNKTVTCYDTTSNDYLRSYFIYSMCTTVAMFCIPLVLILGCYGLIVKALIYNDLDNSPLRRKSIYLVIIVLTVFAVSYIPFHVMKTMNLRARLDFQTPEMCDFNDRVYATYQVTRGLASLNSCVDPILYFLAGDTFRRRLSRATRKASRRSEANLQSKSEEMTLNILSEFKQNGDTSL

SEQ ID NO: 5 - DNA, 3204 bp, Rattus norvegicus
  rat purinergic receptor P2Y, G-protein coupled 1 (P2RY1) cDNA
gaattcgccgccgcctgacttcatggactgcgagcggggtgaagggtagctccagggtccgcggccaagagctctcccaaggctgagtgcgggctgagaatgcggccggaagaagagtcgcggcgtccccggtgacggtccggagttgcaaggtagggagtaggcagatggccaggcaaagttgatcggagcgaggaggtggttggcctgcgccttcggccgctgagaaccgcggagctacttggacttgaccctactcgccccagcgctttgagcagcgcctatctgtcgccctggcgggagcgcacttgcaaacttggtggaggtgccctgcgcgtcctgttgtgtaacctccgtgccgccagctggaccgggaagttgcccgcccgtccttcagcttggatcggtcgcagccccgggcgaatttcatggccccgcgacaaacgcgcggccagagccggtgtgggcgagcccccgcgcgctccgacccgtagggaactcccgcacgcggtccctgcccctggcccgcgcctccgatgcgcgctgagcctctcgccagcagctgccctctcgtcgcggtctgtcctttcgagtagctgcctgagttggaaagaagaggatgaccgaggtgccttggtcggctgtccccaacgggacagatgctgccttcctggctggcctgggctccctttggggaaacagtacaatcgcctcgactgcagcagtttcctcttcattccgatgtgccctgatcaagaccggcttccagttctactacctgcctgcggtctacatcttagtgttcatcataggcttccttggcaacagcgtggcaatctggatgtttgttttccacatgaagccttggagcggcatctcggtgtacatgttcaatttggctctggccgactttttgtatgtgctcaccctaccagctctcatcttctactacttcaacaagactgactggatcttcggggatgttatgtgcaagctgcagaggttcatcttccatgtaaacctctatggcagcatcttgttcctcacctgcatcagtgcacacaggtacagtggcgtggtgtaccctctcaagtctctgggcaggctcaagaagaagaatgccatttatgtcagtgtgctggtatggctcattgtggtggtggccatctcccccattctcttctactctggcactgggattcggaaaaacaaaactgtcacctgctacgactccacgtcagatgagtacctgcgaagttatttcatctacagtatgtgcacgactgtggccatgttctgcatccccctggtgctgatcttgggctgttatggattaattgttagagctttgatctacaaagatctggacaactctcctctccggaggaaatccatttacctggtgataattgtcctgacggtgtttgctgtgtcttacatccctttccatgtgatgaaaacgatgaatttgagggcacggctggatttccagaccccagaaatgtgtgatttcaacgacagggtttatgccacctatcaggtaacaagaggtctagcaagtctcaacagctgtgtggaccccattctttatttcttggctggagatacattcagaaggagactgtcccgagccaccaggaaagcttccaggaggagtgaggccaatttacaatccaagagtgaagaaatgactctcaacattttgtctgagttcaagcagaacggagacacaagtttgtgaaggcaggagatccaagctcctcagtttgtaacatggtcacaagaagtccctgagatgacctgatgcacaggtcaagaagaagcaacattctattggccaaaagaccagtttgtgccacatgagtaggtgttttcatataatgacagttatgttgacattttgttgaaagacacctgaaactgtaagatgtagcaagtgactcttcacactacaaagatgtcttcattccaagctcaagggtttgttttccttgttgctatctgctgtttattccttacaaagtactgtctcggactctttgatggttgctttgaacctgtattgaacggaaatgataaattaagaagaaacttagcacacaagacagaattcaaaattaaaaccgtgatgtgaccactgagaagacatttggaagcccattcttcatgaacagaaaaaggcgacaggacaccaactgttgatgccataggtgctgtggtaagtcttgaaaagcagtgagaactgtttggaatctgcttctgtgcttcctaggtctctccagtctagtccttggagaaagttctcaatctccaccgactgtggaacggacaagttgaaacctgtaaatctctgctcacccaaccaggacactaatccatcttgatatgacaaatatcagtactcagtagcaactagaattcaattagtgacaggacatagacagtgtgatactagtaaatgtgtattgaagcttttcttccattggtcgaatgtaattattgaaatagtatttattagaagaggtttataggccttcagatgtacagattgtaaaacttccttgaagatacaacatactggtgtaaactctatttattgaccagtgcataatgcttcaaatgatcacatgtatattttacagtatatatagctagataaaaatgagaattatatttaagagttttttttccagatattagtatctttgaacctttaggaccagtttgtatttgaatctgaaaagatgaaaatgtgttttctaacaagggttttaattttccctagatttgaatttgcatgctttcctttgtgtgggtttattgctacccagatatgatttggctgttagaccatggtaagatttataacaagcataggatattttagacaggttatgtatatgtacattatatatgtttatacaatagaaagtttgtgttgtcttttcagaaagtttagagtattgccctaactatgatgcagcttccactgccaaaggctaatggcttatctcctattcaaaattgatttagatgtctagtggggttgggtagtgtagatacaatgtgagaatgtaaaagacaatctttagtgatgcagggtgataaagatgcagctgatagttttctgccagatcttgaagctttacaatgtatttttttcttaaggccacatagtacaatcttctttgcggccgcgaattc

SEQ ID NO: 6 - protein, 373 aa, Rattus norvegicus
  rat purinergic receptor P2Y, G-protein coupled 1 (P2RY1)
MTEVPWSAVPNGTDAAFLAGLGSLWGNSTIASTAAVSSSFRCALIKTGFQFYYLPAVYILVFIIGFLGNSVAIWMFVFHMKPWSGISVYMFNLALADFLYVLTLPALIFYYFNKTDWIFGDVMCKLQRFIFHVNLYGSILFLTCISAHRYSGVVYPLKSLGRLKKKNAIYVSVLVWLIVVVAISPILFYSGTGIRKNKTVTCYDSTSDEYLRSYFIYSMCTTVAMFCIPLVLILGCYGLIVRALIYKDLDNSPLRRKSIYLVIIVLTVFAVSYIPFHVMKTMNLRARLDFQTPEMCDFNDRVYATYQVTRGLASLNSCVDPILYFLAGDTFRRRLSRATRKASRRSEANLQSKSEEMTLNILSEFKQNGDTSL

SEQ ID NO: 7 - DNA, 1003 bp, Homo sapiens
  human putative tyrosine phosphatase-like, member a (PTPLA) cDNA
tatacacagcccagccagcacccaggtccttccatccttctgctgcacatggggcgcctgacggaagcggcggcagcgggcagcggctctcgggctgcaggctgggcagggtcccctcccacgctcctgccgctgtctcccacgtcccccaggtgcgcggccaccatggcgtccagcgacgaggacggcaccaacggcggcgcctcggaggccggcgaggaccgggaggctcccggcaagcggaggcgcctggggttgttggccaccgcctggctcaccttctacaacatcgccatgaccgcggggtggttggttctagctattgccatggtacgtttttatatggaaaagggaacacacagagggttatataaaagtattcagaagacacttaaatttttccaaacatttgccttgcttgagatagttcactgtttaattggaattgtacctacttctgtgattgtgactggggtccaagtgagctcaagaatctttatggtgtggctcattactcacagtataaaaccaatccagaatgaagagagtgtggtgctttttctggtcgcgtggactgtgacagagatcactcgctattccttctacacattcagccttcttgaccacttgccatacttcattaaatgggccagatataatttttttatcatcttatatcctgttggagttgctggtgaacttcttacaatatacgctgccttgccgtatgtgaagaaaacaggaatgttttcaataagacttcctaacaaatacaatgtctcttttgactactattattttcttcttataaccatggcatcatatatacctttgtttccacaactctattttcatatgttacgtcaaagaagaaaggtgcttcatggagaggtgattgtagaaaaggatgattaaatgatctctgcaaacaaggtgctttttccagaataaccaagattacctgagtccaagttttaataacaagaataaacaactttgtgaa

SEQ ID NO: 8 - protein, 288 aa, Homo sapiens
  human putative tyrosine phosphatase-like, member a (PTPLA)
MGRLTEAAAAGSGSRAAGWAGSPPTLLPLSPTSPRCAATMASSDEDGTNGGASEAGEDREAPGKRRRLGLLATAWLTFYNIAMTAGWLVLAIAMVRFYMEKGTHRGLYKSIQKTLKFFQTFALLEIVHCLIGIVPTSVIVTGVQVSSRIFMVWLITHSIKPIQNEESVVLFLVAWTVTEITRYSFYTFSLLDHLPYFIKWARYNFFIILYPVGVAGELLTIYAALPYVKKTGMFSIRLPNKYNVSFDYYYFLLITMASYIPLFPQLYFHMLRQRRKVLHGEVIVEKDD

SEQ ID NO: 9 - DNA, 1079 bp, Mus musculus
  mouse putative tyrosine phosphatase-like, member a (PTPLA) cDNA
ggactttattcaaaccgttgccaaatgtgaggcggagggaggtctcactctccaataggcatcaaggacagagtttaaggatgttttggggacaaagaagacactgcccttgaggtatggggaaaggtgattggaggcagggcagagtagagatgccttgtgcacacgtgagcagacttcataagacatgcgtacaggtccgcgtgcgcgtcaccatggcgtccagtgaggaggacggcaccaacggcgcctccgaggccagcgacgagaaggaagccgccggcaagcggagacgcctaggcttactggccaccgcctggctcaccttctacaatatcgccatgacggccgggtggttggttcttgctattgctatggtacgcttttatatggaaaaaggaacacacagaggtttatataaaagcattcagaagacacttaagtttttccaaacatttgctttgcttgaggtagtccattgtctgatcggaattgtacccacttctgtgcttgtgactggggtccaagtgagctcaagaatcttcatggtgtggctcattactcacagtataaaacccatccagaatgaagagagcgtggtgctttttctggtctcctggactgtgaccgagatcactcgctattccttctacacattcagtctcctcgaccacttgccgcacttcattaaatgggccagatacaatttgtttatcatcttatatcccgttggagttgctggggaacttctcacaatatacgccgccttgccttacgtaaagaagtcaggaatgttctcagtacggcttcccaacaagtacaatgtttcttttgactactactattttcttctcataaccatggcctcctatataccgttgtttcctcagctctattttcatatgttacgtcagagaagaaaggtgctccacggggaggtgatcgcggagaaggacgattaagtggttcccacacacaaggtgctcttttcagaaaaaccggattacttgagtccaagttttaataataagaataaacgacttcatgaaataccaaaaaaaaaaaaaaaaaaaaaaaaa

SEQ ID NO: 10 - protein, 281 aa, Mus musculus
  mouse putative tyrosine phosphatase-like, member a (PTPLA)
MGKGDWRQGRVEMPCAHVSRLHKTCVQVRVRVTMASSEEDGTNGASEASDEKEAAGKRRRLGLLATAWLTFYNIAMTAGWLVLAIAMVRFYMEKGTHRGLYKSIQKTLKFFQTFALLEVVHCLIGIVPTSVLVTGVQVSSRIFMVWLITHSIKPIQNEESVVLFLVSWTVTEITRYSFYTFSLLDHLPHFIKWARYNLFIILYPVGVAGELLTIYAALPYVKKSGMFSVRLPNKYNVSFDYYYFLLITMASYIPLFPQLYFHMLRQRRKVLHGEVIAEKDD

SEQ ID NO: 11 - DNA, 5601 bp, Homo sapiens
  human cysteine-rich repeat-containing protein S52 precursor, cysteine-rich motorneuron 1 (CRIM1) cDNA
ggcccggctgcgaggaggaggcggcggcggcgcaggaggatgtacttggtggcgggggacagggggttggccggctgcgggcacctcctggtctcgctgctggggctgctgctgctgctggcgcgctccggcacccgggcgctggtctgcctgccctgtgacgagtccaagtgcgaggagcccaggaactgcccggggagcatcgtgcagggcgtctgcggctgctgctacacgtgcgccagccagaggaacgagagctgcggcggcaccttcgggatttacggaacctgcgaccgggggctgcgttgtgtcatccgccccccgctcaatggcgactccctcaccgagtacgaagcgggcgtttgcgaagatgagaactggactgatgaccaactgcttggttttaaaccatgcaatgaaaaccttattgctggctgcaatataatcaatgggaaatgtgaatgtaacaccattcgaacctgcagcaatccctttgagtttccaagtcaggatatgtgcctttcagctttaaagagaattgaagaagagaagccagattgctccaaggcccgctgtgaagtccagttctctccacgttgtcctgaagattctgttctgatcgagggttatgctcctcctggggagtgctgtcccttacccagccgctgcgtgtgcaaccccgcaggctgtctgcgcaaagtctgccagccgggaaacctgaacatactagtgtcaaaagcctcagggaagccgggagagtgctgtgacctctatgagtgcaaaccagttttcggcgtggactgcaggactgtggaatgccctcctgttcagcagaccgcgtgtcccccggacagctatgaaactcaagtcagactaactgcagatggttgctgtactttgccaacaagatgcgagtgtctctctggcttatgtggtttccccgtgtgtgaggtgggatccactccccgcatagtctctcgtggcgatgggacacctggaaagtgctgtgatgtctttgaatgtgttaatgatacaaagccagcctgcgtatttaacaatgtggaatattatgatggagacatgtttcgaatggacaactgtcggttctgtcgatgccaagggggcgttgccatctgcttcactgcccagtgtggtgagataaactgcgagaggtactacgtgcccgaaggagagtgctgcccagtgtgtgaagatccagtgtatccttttaataatcccgctggctgctatgccaatggcctgatccttgcccacggagaccggtggcgggaagacgactgcacattctgccagtgcgtcaacggtgaacgccactgcgttgcgaccgtctgcggacagacctgcacaaaccctgtgaaagtgcctggggagtgttgccctgtgtgcgaagaaccaaccatcatcacagttgatccacctgcatgtggggagttatcaaactgcactctgacagggaaggactgcattaatggtttcaaacgcgatcacaatggttgtcggacctgtcagtgcataaacaccgaggaactatgttcagaacgtaaacaaggctgcaccttgaactgtcccttcggtttccttactgatgcccaaaactgtgagatctgtgagtgccgcccaaggcccaagaagtgcagacccataatctgtgacaagtattgtccacttggattgctgaagaataagcacggctgtgacatctgtcgctgtaagaaatgtccagagctctcatgcagtaagatctgccccttgggtttccagcaggacagtcacggctgtcttatctgcaagtgcagagaggcctctgcttcagctgggccacccatcctgtcgggcacttgtctcaccgtggatggtcatcatcataaaaatgaggagagctggcacgatgggtgccgggaatgctactgtctcaatggacgggaaatgtgtgccctgatcacctgcccggtgcctgcctgtggcaaccccaccattcaccctggacagtgctgcccatcatgtgcagatgactttgtggtgcagaagccagagctcagtactccctccatttgccacgcccctggaggagaatactttgtggaaggagaaacgtggaacattgactcctgtactcagtgcacctgccacagcggacgggtgctgtgtgagacagaggtgtgcccaccgctgctctgccagaacccctcacgcacccaggattcctgctgcccacagtgtacagatcaaccttttcggccttccttgtcccgcaataacagcgtacctaattactgcaaaaatgatgaaggggatatattcctggcagctgagtcctggaagcctgacgtttgtaccagctgcatctgcattgatagcgtaattagctgtttctctgagtcctgcccttctgtatcctgtgaaagacctgtcttgagaaaaggccagtgttgtccctactgcatagaagacacaattccaaagaaggtggtgtgccacttcagtgggaaggcctatgccgacgaggagcggtgggaccttgacagctgcacccactgctactgcctgcagggccagaccctctgctcgaccgtcagctgcccccctctgccctgtgttgagcccatcaacgtggaaggaagttgctgcccaatgtgtccagaaatgtatgtcccagaaccaaccaatatacccattgagaagacaaaccatcgaggagaggttgacctggaggttcccctgtggcccacgcctagtgaaaatgatatcgtccatctccctagagatatgggtcacctccaggtagattacagagataacaggctgcacccaagtgaagattcttcactggactccattgcctcagttgtggttcccataattatatgcctctctattataatagcattcctattcatcaatcagaagaaacagtggataccactgctttgctggtatcgaacaccaactaagccttcttccttaaataatcagctagtatctgtggactgcaagaaaggaaccagagtccaggtggacagttcccagagaatgctaagaattgcagaaccagatgcaagattcagtggcttctacagcatgcaaaaacagaaccatctacaggcagacaatttctaccaaacagtgtgaagaaaggcaactaggatgaggtttcaaaagacggaagacgactaaatctgctctaaaaagtaaactagaatttgtgcacttgcttagtggattgtattggattgtgacttgatgtacagcgctaagaccttactgggatgggctctgtctacagcaatgtgcagaacaagcattcccacttttcctcaagataactgaccaagtgttttcttagaaccaaagtttttaaagttgctaagatatatttgcctgtaagatagctgtagagatatttggggtggggacagtgagtttggatggggaaatgggtgggagggtggtgttgggaagaaaaattggtcagcttggctcggggagaaacctggtaacataaaagcagttcagtggcccagaggttatttttttcctattgctctgaagactgcactggttgctgcaaagctcaggcctgaatgagcaggaaacaaaaaaggccttgcgacccagctgccataaccaccttagaactaccagacgagcacatcagaaccctttgacagccatcccaggtctaaagccacaagtttcttttctatacagtcacaactgcagtaggcagtgaggaagccagagaaatgcgatagcggcatttctctaaagcgggttattaaggatatatacagttacactttttgctgcttttattttcttccaagccaatcaatcagccagttcctagcagagtcagcacatgaacaagatctaagtcatttcttgatgtgagcactggagcttttttttttttacaacgtgacaggaagaggagggagagggtgacgaacaccaggcatttccaggggctatatttcactgtttgttgttgctttgttctgttatattgttggttgttcatagtttttgttgaagctctagcttaagaagaaactttttttaaaaagactgtttggggattctttttccttattatatactgattctacaaaatagaaactacttcattttaattgtatattattcaagcacctttgttgaagctcaaaaaaaatgatgcctctttaaactttagcaattataggagtatttatgtaactatcttatgcttcaaaaaacaaaagtatttgtgtgcatgtgtatataatatatatatatacatatatatttatacacatacaatttatgttttcctgttgaatgtatttttatgagattttaaccagaacaaaggcagataaacaggcattccatagcagtgcttttgatcacttacaaattttttgaataacacaaaatctcattctacctgcagtttaattggaaagatgtgtgtgtgagagtatgtatgtgtgtgtgtgtgtgtgtgtgtgtgcgcgcgcacgcacgccttgagcagtcagcattgcacctgctatggagaagggtattcctttattaaaatcttcctcatttggatttgctttcagttggttttcaatttgctcactggccagagacattgatggcagttcttatctgcatcactaatcagctcctggattttttttttttttttttcaaacaatggtttgaaacaactactggaatattgtccacaataagctggaagtttgttgtagtatgcctcaaatataactgactgtatactatagtggtaacttttcaaacagcccttagcacttttatactaattaacccatttgtgcattgagttttcttttaaaaatgcttgttgtgaaagacacagatacccagtatgcttaacgtgaaaagaaaatgtgttctgttttgtaaaggaactttcaagtattgttgtaaatacttggacagaggttgctgaactttaaaaaaaattaatttattattataatgacctaatttattaatctgaagattaaccatttttttgtcttagaatatcaaaaagaaaaagaaaaaggtgttctagctgtttgcatcaaaggaaaaaaagatttattatcaaggggcaatatttttatcttttccaaaataaatttgttaatgatacattacaaaaatagattgacatcagcctgattagtataaattttgttggtaattaatccattcctggcataaaaagtctttatcaaaaaaaattgtagatgcttgctttttgttttttcaatcatggccatattatgaaaatactaacaggatataggacaaggtgtaaatttttttattattattttaaagatatgatttatcctgagtgctgtatctattactcttttactttggttcctgttgtgctcttgtaaaagaaaaatataatttcctgaagaataaaatagatatatggcacttggagtgcatcatagttctacagtttgtttttgttttcttcaaaaaagctgtaagagaattatctgcaacttgattcttggcaggaaataaacattttgagttgaaatcaaaaaaaaaaaaaaaaaa

SEQ ID NO: 12 - protein, 1036 aa, Homo sapiens
  human cysteine-rich repeat-containing protein S52 precursor, cysteine-rich motorneuron 1 (CRIM1)
MYLVAGDRGLAGCGHLLVSLLGLLLLLARSGTRALVCLPCDESKCEEPRNCPGSIVQGVCGCCYTCASQRNESCGGTFGIYGTCDRGLRCVIRPPLNGDSLTEYEAGVCEDENWTDDQLLGFKPCNENLIAGCNIINGKCECNTIRTCSNPFEFPSQDMCLSALKRIEEEKPDCSKARCEVQFSPRCPEDSVLIEGYAPPGECCPLPSRCVCNPAGCLRKVCQPGNLNILVSKASGKPGECCDLYECKPVFGVDCRTVECPPVQQTACPPDSYETQVRLTADGCCTLPTRCECLSGLCGFPVCEVGSTPRIVSRGDGTPGKCCDVFECVNDTKPACVFNNVEYYDGDMFRMDNCRFCRCQGGVAICFTAQCGEINCERYYVPEGECCPVCEDPVYPFNNPAGCYANGLILAHGDRWREDDCTFCQCVNGERHCVATVCGQTCTNPVKVPGECCPVCEEPTIITVDPPACGELSNCTLTGKDCINGFKRDHNGCRTCQCINTEELCSERKQGCTLNCPFGFLTDAQNCEICECRPRPKKCRPIICDKYCPLGLLKNKHGCDICRCKKCPELSCSKICPLGFQQDSHGCLICKCREASASAGPPILSGTCLTVDGHHHKNEESWHDGCRECYCLNGREMCALITCPVPACGNPTIHPGQCCPSCADDFVVQKPELSTPSICHAPGGEYFVEGETWNIDSCTQCTCHSGRVLCETEVCPPLLCQNPSRTQDSCCPQCTDQPFRPSLSRNNSVPNYCKNDEGDIFLAAESWKPDVCTSCICIDSVISCFSESCPSVSCERPVLRKGQCCPYCIEDTIPKKVVCHFSGKAYADEERWDLDSCTHCYCLQGQTLCSTVSCPPLPCVEPINVEGSCCPMCPEMYVPEPTNIPIEKTNHRGEVDLEVPLWPTPSENDIVHLPRDMGHLQVDYRDNRLHPSEDSSLDSIASVVVPIIICLSIIIAFLFINQKKQWIPLLCWYRTPTKPSSLNNQLVSVDCKKGTRVQVDSSQRMLRIAEPDARFSGFYSMQKQNHLQADNFYQTV

SEQ ID NO: 13 - DNA, 4012 bp, Mus musculus
  mouse cysteine-rich repeat-containing protein, cysteine-rich motorneuron 1 (CRIM1) partial cDNA
ctggccggctgcgggcacctctcggtctcgctgctggggctgctgctgctgctggcgcgctcaggcacccgggcgctggtctgcctgccctgtgacgagtccaagtgcgaggagcctcggagctgcccaggaagcatcgtgcagggcgtctgcggctgctgctacatgtgcgcccgccagaggaacgagagctgcggtggagcctatgggctccatggagcctgcgaccgggggctgcgctgtgtcatccgccccccgctcaatggcgactccatcaccgagtacgaagtgggcgtctgcgaagatgaggactgggatgatgaccagctaataggttttgaaccctgcaatgaaaacctcatctccggctgcaacataatcaatgggaaatgcgaatgtggtaccatccgaacctgcaacaatccctttgagtttccaaggaaggacatgtgcctttcagcattaaagaggatcgaagaagagaagccagattgcagcaaggcccgctgtgaagtgcggttctctccacgttgccctgaagattccattctgatcgagggctatgctccccccggggagtgctgtcctttacccagccgctgcgtgtgcgaccctgcgggctgtctgcgcaaagtctgccagccaggatacctgaacattctagtgtccaaagcctcagggaagccgggagagtgttgtgacctctatgagtgtaaaccagttttcagcgtggactgcagcaccgtggagtgcccccctgtccagcaggccgtgtgccccctggacagctacgaaacgcaagtgcggctcacagcggatggctgctgtaccctgccagcaagatgcgagtgtctctctggcttatgtggttttcccgtttgtgaggtgggatctactccccgaatagtctctcgtggagatgggacacctggaaagtgctgtgatgtctttgaatgtgttaatgaaacaaagccagcctgcgtgttcaacagcgtggagtattacgacggagacatgtttcgaatggacaactgtcggttctgccgatgccaggggggtgtctccatctgcttcacggcgcagtgtggggaactgaactgcgaaagatactatgtgcctgagggggagtgttgccctgtgtgtgaagatcccatctatcctcttaacaaccctgctggctgctatgccaatggccagatccgcgcccacggggaccggtggcgggaagatgactgtaccttctgccagtgtatcaacggagaacctcactgcgtggccacggcctgcgggcagagctgcatgcacccagtgaaagtgcccggggagtgttgccccgtgtgtgaagaaccaacctacatcacgattgatccacctgcatgcggggagttgtcaaactgttctctgaaggagaaggactgcgtttatggcttcaaactggatcacaatggctgtcgaacctgtcagtgcaaaatcagggaggaactctgcttaggcctcaaaagggcctgcaccctggactgtccctttggcttcctcaccgatgtgcacaactgtgaactctgtcagtgccgcccacggcccaagaagtgcagaccaacaatgtgcgacaagttttgtccgcttggattcctgaagaataagcatggctgtgacatctgtcggtgtaagaaatgtccagagctgccatgcagcaagatctgccccttgggcttccagcaggacagtcatggctgtcttatctgcaagtgccgagaggtccctccttcagccgggccacctgtcctgtcaggcacatgtctgtccatggatggccatcatcataagaacgaggagagctggcatgatggttgccgggaatgctactgtcacaacggaaaggaaatgtgcgctctcatcacctgtcccgtgcctgcctgcggcaaccccaccattcgttccggacagtgctgcccgtcttgcacagatgactttgtagtgcagaagccggagctcagcaccccttctatttgccacgcccccggaggagagtactttgtggaaggggaaacttggaacattgactcctgtacacagtgcacctgtcacagtggtcgagtgctgtgtgagacggaggtgtgcccaccattgctctgccagaacccctcccgcacccaggactcctgctgcccacagtgtacagatgaccctcctcagccttccacatcccataatgagagcgtgcctagctactgcaggaatgatgaaggagatatcttcctggcggctgagtcctggaagcccgacgcctgcaccagctgcgtgtgcgtggatagcgcaattagctgctactctgagtcttgcccttccgtggcctgtgaaagacctgttttgaggaaaggccagtgttgcccctactgcttagaagacacaattccaaagaaagtggtgtgtcacttcagtgggaagacctatgctgacgaggaacggtgggatattgacagctgcacrcactgctactgcctgcaaggccagaccctctgctcgaccgtcagctgcccaccgttaccctgtgccgagcccatcaaggtggaagggagttgctgcccaatgtgcccagaaatgtatgtgccagagccaaccaatgtacccattgagaagaaaaatcatcgtggcgagattgacctggaggtccccatgtggcccaccccaagtgaaaatgacatcatccatctccctagagatatgggtcacctccaggtagattacagagataataacaggctgcatccaggcgaagactcatcactggactccattgtctccgttgtggttcccataataatatgcctgtccatcatcatagctttcctgctcatcaaccagaagaagcagtgggtaccactgctgtgctggtaccggacaccaaccaagccttcttccttgaataatcagctggtatctgtggactgcaagaaaggcactcgagtccaggtggatggtccccagagaatgctaagaattgccgaaccagatgctcgattcagtggcttctacagcatgcagaaacagaaccatctacaggcagacaacttctaccaaacggtgtgaacacgggcagcagcgccgatgaggtcccgaaagacggagaaagacgaaaatctgctctttaaagtaaactaggatttgcgcacttgcttagtgtcttgtgtcagattgcgacttggtgtccagcgctaggggctggactgggatgggctctgtctacagcactgtgcagaacaagcattcccacttggcctcaagataactgaccacatgttttcatagaaccaaagttttaaaacttgctaaagtatatttgcttgtaagatagctgtagagacatttgggggaggggagaaagagtttggtgggggaaatgggttgggagggtagtgttgggaagaaaaaaaaatggtcagcttggctcggggagaaagccagtaaaaaaataaaagcaatttatggcccagaggaatttctttttcctgttactntcaagactgttggttgctgcaaaggtagggcctgaatgagcaggatcacaaaaggccttgcaaccgagctgccataccacaccagacaagcgcatcagaactctcgatagccatcccaggtctaaagccacaagtttctatagagtcacagccgcagtaggcagtgaggaagccagggagatggaaagcaacagttctcgaaagcgggttcttgaggatgtattcactttttttgctgctgctgttttcttccgtgccaaccagccagttcctaggagacacagcaggttgagtaggaacgaggtcacctcttgacctgaccactggagctttgctcgctacgtgacaggaagggcaacggcgaaggacaccaggcatttccaggggctacacttcattgttccttgttgttttcttctgtgctatcattggttgttcatagttttgttaaagctctagcttaagaagaaacttttgagaaaaaaaaaaaaaaaaaa

SEQ ID NO: 14 - protein, 1028 aa, Mus musculus
  mouse cysteine-rich repeat-containing protein, cysteine-rich motorneuron 1 (CRIM1), partial
LAGCGHLSVSLLGLLLLLARSGTRALVCLPCDESKCEEPRSCPGSIVQGVCGCCYMCARQRNESCGGAYGLHGACDRGLRCVIRPPLNGDSITEYEVGVCEDEDWDDDQLIGFEPCNENLISGCNIINGKCECGTIRTCNNPFEFPRKDMCLSALKRIEEEKPDCSKARCEVRFSPRCPEDSILIEGYAPPGECCPLPSRCVCDPAGCLRKVCQPGYLNILVSKASGKPGECCDLYECKPVFSVDCSTVECPPVQQAVCPLDSYETQVRLTADGCCTLPARCECLSGLCGFPVCEVGSTPRIVSRGDGTPGKCCDVFECVNETKPACVFNSVEYYDGDMFRMDNCRFCRCQGGVSICFTAQCGELNCERYYVPEGECCPVCEDPIYPLNNPAGCYANGQIRAHGDRWREDDCTFCQCINGEPHCVATACGQSCMHPVKVPGECCPVCEEPTYITIDPPACGELSNCSLKEKDCVYGFKLDHNGCRTCQCKIREELCLGLKRACTLDCPFGFLTDVHNCELCQCRPRPKKCRPTMCDKFCPLGFLKNKHGCDICRCKKCPELPCSKICPLGFQQDSHGCLICKCREVPPSAGPPVLSGTCLSMDGHHHKNEESWHDGCRECYCHNGKEMCALITCPVPACGNPTIRSGQCCPSCTDDFVVQKPELSTPSICHAPGGEYFVEGETWNIDSCTQCTCHSGRVLCETEVCPPLLCQNPSRTQDSCCPQCTDDPPQPSTSHNESVPSYCRNDEGDIFLAAESWKPDACTSCVCVDSAISCYSESCPSVACERPVLRKGQCCPYCLEDTIPKKVVCHFSGKTYADEERWDIDSCTHCYCLQGQTLCSTVSCPPLPCAEPIKVEGSCCPMCPEMYVPEPTNVPIEKKNHRGEIDLEVPMWPTPSENDIIHLPRDMGHLQVDYRDNNRLHPGEDSSLDSIVSVVVPIIICLSIIIAFLLINQKKQWVPLLCWYRTPTKPSSLNNQLVSVDCKKGTRVQVDGPQRMLRIAEPDARFSGFYSMQKQNHLQADNFYQTV

SEQ ID NO: 15 - DNA, 2276 bp, Homo sapiens
  human calmodulin-dependent phosphoprotein phosphatase catalytic subunit, calcineurin A alpha (PPP3CA) cDNA
ccagctcagagcctagacctccagccgagcggtttgcagccgcgggcggcggcggcggcggcggcgttgagtgtctggcccgccggtccggtcggggtgtgcagtcggacggacgagcagcgcgtcgctgtcctccggcagctggagatgtccgagcccaaggcaattgatcccaagttgtcgacgaccgacagggtggtgaaagctgttccatttcctccaagtcaccggcttacagcaaaagaagtgtttgataatgatggaaaacctcgtgtggatatcttaaaggcgcatcttatgaaggagggaaggctggaagagagtgttgcattgagaataataacagagggtgcatcaattcttcgacaggaaaaaaatttgctggatattgatgcgccagtcactgtttgtggggacattcatggacaattctttgatttgatgaagctctttgaagtcgggggatctcctgccaacactcgctacctcttcttaggggactatgttgacagagggtacttcagtattgaatgtgtgctgtatttgtgggccttgaaaattctctaccccaaaacactgtttttacttcgtggaaatcatgaatgtagacatctaacagagtatttcacatttaaacaagaatgtaaaataaagtattcagaacgagtatatgatgcctgtatggatgcctttgactgccttcccctggctgccctgatgaaccaacagttcctgtgtgtgcatggtggtttgtctccagagattaacactttagatgatatcagaaaattagaccgattcaaagaaccacctgcatatggacctatgtgtgatatcctgtggtcagaccccctggaagattttggaaatgagaagactcaggaacatttcactcacaacacagtcagggggtgttcatacttctacagttacccggctgtatgtgaattcttacagcacaataacttgttatctatactccgagcccacgaagcccaagatgcagggtaccgcatgtacaggaaaagccaaacaacaggcttcccttctctaattacaattttttcagcaccaaattacttagatgtatacaataacaaagctgcagtattgaagtatgagaacaatgttatgaatatcaggcaattcaactgttctcctcatccatactggcttccaaatttcatggatgtttttacttggtcccttccatttgttggggaaaaagtgactgagatgctggtaaatgtcctcaacatctgctcagatgatgaactagggtcagaagaagatggatttgatggtgcaacagctgcagcccggaaagaggtgataaggaacaagatccgagcaataggcaaaatggccagagtgttctcagtgctcagagaagagagtgagagtgtgctgacgctgaaaggcttgaccccaactggcatgctccccagcggagtactttctggagggaagcaaaccctgcaaagcgctactgttgaggctattgaggctgatgaagctatcaaaggattttcaccacaacataagatcactagcttcgaggaagccaagggcttagaccgaattaatgagaggatgccgcctcgcagagatgccatgccctctgacgccaaccttaactccatcaacaaggctctcacctcagagactaacggcacggacagcaatggcagtaatagcagcaatattcagtgaccacttcctgttcacattttttttttttttttttttttttttttttttgagctgcggggcatgatggggattgctgcatatcagcagttggatgttcttgcctctgacagtagcttatttgctctgggggccaggaattggattcagtttacactatcattaaaaaagagggagagagataataaactatattttggtggggatggtgattaaacacctcttttgggtatgccttttaaaaatgcttatagagaaaaaaaattttaaaaaaagaaagctaatgctagtatatactgcaatgttaggggaatgaacatgttttcctactgcattggggacttctagataggttaatgaaaggccttttattctgttactggacatgaaaactttgtctaatttcttactctattgtacgtttacagtcgcagcactaaaaatggatgacatcaaacatttttaacaaaatgatgatgtacaaactaaggactatttattgataatgttttgctactcttgtcagacaatggctataaactgaattaggcagtcttaaaaaaaaa

SEQ ID NO: 16 - protein, 521 aa, Homo sapiens
  human calmodulin-dependent phosphoprotein phosphatase catalytic subunit, calcineurin A alpha (PPP3CA)
MSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFMDVFTWSLPFVGEKVTEMLVNVLNICSDDELGSEEDGFDGATAAARKEVIRNKIRAIGKMARVFSVLREESESVLTLKGLTPTGMLPSGVLSGGKQTLQSATVEAIEADEAIKGFSPQHKITSFEEAKGLDRINERMPPRRDAMPSDANLNSINKALTSETNGTDSNGSNSSNIQ

SEQ ID NO: 17 - DNA, 2194 bp, Mus musculus
  mouse calmodulin-dependent phosphoprotein phosphatase catalytic subunit, calcineurin A alpha (PPP3CA) cDNA
gcgccggtgcggtcggggtgtgcagtcggacgggacgagcagcgcgtcgctgtccccccctcccggtggctggagatgtccgagcccaaggcgattgatcccaagttgtcgacgaccgacagggtggtgaaagccgttccatttccaccaagtcaccggctgacagcaaaggaagtgtttgataatgatgggaaacctcgtgtggatatcttaaaagcacatctcatgaaggagggcaggctggaagaaagtgttgcattgagaataataacagagggtgcttcgattctccgacaggaaaaaaacttgctggatatcgacgcaccagtcacagtttgtggggacatccatggacaattctttgacttgatgaagctctttgaagtgggaggatctcctgccaacactcgctacctcttcttaggggactatgttgacagagggtacttcagtatcgaatgtgtgctgtatttgtgggccttgaaaattctttaccccaaaacactgtttttacttcgcggaaaccatgaatgtaggcacctcacagagtatttcacgtttaaacaagaatgtaaaataaagtattcagaacgcgtttatgacgcctgtatggatgccttcgactgccttcccctggctgcgctaatgaaccagcagttcctgtgtgtacacggtggtttgtctccagagattaacactctagatgacatcagaaaattagaccgattcaaagaaccacctgcttatgggcccatgtgtgacatcctatggtcagaccccctggaggactttggaaatgagaagactcaggaacatttcactcacaacacagtcagaggctgttcgtacttctacagttacccagctgtgtgtgacttcctgcagcacaataatttgttgtccatactccgcgcccacgaagcccaggatgcagggtaccgcatgtacaggaaaagccaaacaacaggcttcccgtctctaattacaatcttctcggcaccaaattacttagatgtgtacaataacaaagctgcagtgttgaagtacgagaacaatgtgatgaacatcaggcagttcaactgctccccgcatccgtactggctcccaaatttcatggatgttttcacctggtcgctgccatttgttggggagaaagtgactgagatgctggtcaatgttctcaacatctgctccgacgatgaactggggtcagaagaagatggatttgacggagccacggccgcagcccggaaggaagtcatcagaaacaagatccgagcaataggcaaaatggccagagtgttctcagttctcagagaagagagtgagagtgtcctgacactgaagggcctgaccccaactggcatgctccccagcggagtgctctctggcgggaaacagactctgcaaagcgctactgttgaggctattgaggctgatgaagccatcaaaggattttcaccacaacataagatcactagcttcgaggaggccaagggcttagaccgaattaacgagaggatgccacctcgcagagacgccatgccctctgacgccaaccttaactccatcaacaaggctctcgcctcagagactaacggcacggacagcaatggcagtaatagcagcaatatccagtgaccacttcctgttcacttttttttttttttgagctgcagggcatgatgggattgctgcatctcagcagttggatgttcttgcctctgaaggtagcttgtttgctctgggggccaggaattggattcagtttacactatcatgaaaaataaaaataaaaaaagagggagagagataataaactatattttggtgagggtggtgattaaacacctcttttgggtatgcctttaaaaaatgcttctagggcaaaaaagttttaaaaagaaagctaatgctagctatactgcaatgttaggggaatgaacgcgttttcctactgcactggggacttttagataggttaatgaaaggcctttattctgttactggacacgaaaactttgtctaatttcttatactctattgtacctttacagtcgcagcactaaaatggaagacatcaaacatttttaacagaaaaaaaaaaagatgtaaaaactaactaaggactatttattaatgatgttttgctactcctgtcagacaatggctataaactgaattaggcagtctt

SEQ ID NO: 18 - protein, 521 aa, Mus musculus
  mouse calmodulin-dependent phosphoprotein phosphatase catalytic subunit, calcineurin A alpha (PPP3CA)
MSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCDFLQHNNLLSILRAHEAQDAGYRMYRKSQTTGFPSLITIFSAPNYLDVYNNKAAVLKYENNVMNIRQFNCSPHPYWLPNFMDVFTWSLPFVGEKVTEMLVNVLNICSDDELGSEEDGFDGATAAARKEVIRNKIRAIGKMARVFSVLREESESVLTLKGLTPTGMLPSGVLSGGKQTLQSATVEAIEADEAIKGFSPQHKITSFEEAKGLDRINERMPPRRDAMPSDANLNSINKALASETNGTDSNGSNSSNIQ

SEQ ID NO: 19 - DNA, 2360 bp, Rattus norvegicus
  rat calmodulin-dependent phosphoprotein phosphatase catalytic subunit, calcineurin A alpha (PPP3CA) cDNA
agaaaataaataaataaccagcacacacgcgcagcccggagcgaggcgcggggctggtggcggcggaggaggagtgaaggcggcggcggcggaggagggacgcgcggagccggcagtaactttcgagccagcccagagcccggagctccagccgagcggtttgcagcgcggcggcggcggcgctgagtgtctggcccgccggtgcggtcggggtgtgcagtcggacgggaccagcagcgcgtcgctgtccccccctcccggtgactggagatgtccgagcccaaggcgattgatcccaagttgtcgactacggacagggtggtgaaagccgttccatttccgccaagtcaccggctgacagcaaaggaagtgtttgataacgatgggaagcctcgtgtggatatcttaaaagcacatctcatgaaggaaggcaggctggaagaaagtgtcgcgttgagaataataacagagggtgcttcgattctccgacaggaaaaaaacttgctggatattgatgccccagtcacagtttgcggggacatccatggacaattctttgacttgatgaagctctttgaagtgggaggatctcctgccaacactcgctacctcttcttaggggactatgttgacagagggtacttcagtatcgaatgtgtgctgtatttgtgggccttgaaaattctttaccccaaaacactgtttttacttcgtggaaaccatgaatgtaggcacctaacagagtatttcacgtttaaacaagaatgtaaaataaagtattcagaacgcgtttatgacgcctgtatggatgccttcgactgccttcccctggctgcgctgatgaaccaacaattcctgtgtgtacacggtggtttgtctccagagattaacactctagatgacatcagaaaattagaccgattcaaagaaccacctgcttatgggcctatgtgtgacatcttgtggtcagaccccctggaggactttggaaatgagaagactcaggaacatttcactcacaacacagtcaggggttgttcgtacttctacagttacccggctgtatgtgacttcctgcagcacaataatttgttgtccatactccgagcccacgaagcccaggacgcagggtaccgcatgtacaggaaaagccaaacaactggcttcccgtctctaattacgatcttctcggcaccaaattacttagatgtgtacaataataaagctgcagtgttgaagtacgagaacaacgtgatgaacatcaggcagttcaactgctccccccatccgtactggctcccaaatttcatggatgttttcacctggtcgctgccatttgttggggagaaagtgactgagatgctggtaaacgtcctgaacatctgctcagatgatgaactggggtcagaagaagatggatttgacggagccacggctgcagcccggaaggaggtcatcaggaacaagatccgagcaataggcaaaatggccagagtattctcagttctcagagaagagagtgagagcgttctaactctgaagggcctgaccccgactggcatgctccccagcggagtgctctctggcgggaaacaaactctgcaaagcgccatcaaaggattctcaccacaacataagattaccagcttcgaggaggccaagggcttagaccgaattaacgagaggatgccgcctcgcagagacgccatgccttccgacgccaaccttaactccatcaacaaggctctcgcctcagagactaacggcacagacagcaacggcagtaatagcagcaatattcagtgaccacttcctgttcactttttttttttgagctgcaggcatgatgggtttgctgcatctcagcagttggtgttcttgcctctgacggtagcttgtttgctgctgggggggccaggaattggattcagtttacactatcatgaaaaaaaaaaaagagggagagagagagagataataaaactatattttggtgagggtggtgattaaacacctcttttgggtatgcctttaaaaatgcttctaggaaaaaaaaagttttaaaaagaaagctaatgctagtctatacttcaatgttaggggaatgaacacgttttcctagcgcactggggacttttagataggttaatgaaaggccttttattctgttactggacacgaaaactttgtctaatttcttatactctattgtacgtttacagtcgcagcactaaaaatggatgacatcaaacatttttaaacagaaaaaaaaagatgtgcgggctaaataaccactatttattgataatgttttgctactcttgtcagacaatggctataaactgaattaggccgaattc

SEQ ID NO: 20 - protein, 511 aa, Rattus norvegicus
  rat calmodulin-dependent phosphoprotein phosphatase catalytic subunit, calcineurin A alpha (PPP3CA)
MSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCDFLQHNNLLSILRAHEAQDAGYRMYRKSQTTGFPSLITIFSAPNYLDVYNNKAAVLKYENNVMNIRQFNCSPHPYWLPNFMDVFTWSLPFVGEKVTEMLVNVLNICSDDELGSEEDGFDGATAAARKEVIRNKIRAIGKMARVFSVLREESESVLTLKGLTPTGMLPSGVLSGGKQTLQSAIKGFSPQHKITSFEEAKGLDRINERMPPRRDAMPSDANLNSINKALASETNGTDSNGSNSSNIQ

SEQ ID NO: 21 - DNA, 3984 bp, Homo sapiens
  human protein tyrosine phosphatase, non- receptor type 3 (PTPN3a) cDNA
ctgcaggttattcagcgatagttatgacctcccggttacgtgcgttgggtggaagaattaataatatacgcacctcggagttacccaaagagaaaactcgatcagaagtcatttgcagcatccactttttagatggcgtggtacagacctttaaagttactaaacaagacactggccaggttcttctggatatggtgcacaaccacctgggtgtgactgaaaaggaatattttggtttacagcatgatgacgactccgtggactctcctagatggctggaagcaagcaaacccatcaggaagcagttaaaaggaggtttcccctgtaccctgcattttcgagtaagattttttatacctgatcccaacacactgcagcaagaacaaaccaggcacttgtatttcttacaactgaagatggatatttgcgaaggaaggttaacctgccctcttaactcagcagtggttctagcgtcctatgccgtacaatctcattttggagactataattcttccatacatcatccaggctatctttccgatagtcactttatacccgatcaaaatgaggactttttaacaaaagtcgaatctctgcatgagcagcacagtgggctaaaacaatcagaagcagaatcctgctatatcaacatagcgcggaccctcgacttctatggagtagaactgcacagtggtagggatctgcacaatttagacctaatgattggaattgcttccgcgggtgttgctgtgtaccgaaaatacatttgcacaagtttctatccttgggtgaacattctcaaaatttctttcaaaaggaaaaagttcttcatacatcagcgacagaaacaggctgaatccagggaacatattgtggccttcaacatgctgaattaccgatcttgcaaaaacttgtggaaatcctgtgttgagcaccatacgttctttcaggcaaagaagctactacctcaggaaaagaatgttctgtctcagtactggactatgggctctcggaacaccaaaaagtcggtaaataaccaatattgcaaaaaggtgattggcgggatggtgtggaacccagccatgcggagatccttatcagtggagcacttagaaaccaagagtctgccttctcgttcccctcccattactcccaactggcgaagtcctcggctccggcacgaaatccgaaagccacgccactcttctgcagataaccttgcaaatgaaatgacctacatcacggaaacggaagatgtattttacacgtacaagggctctctggcccctcaagacagcgattctgaagtttctcagaaccgaagcccgcaccaagagagtttatccgagaacaatccggcacaaagctacctgacccagaagtcatccagttctgtgtctccatcttcaaatgctccaggctcctgctcacctgacggcgttgatcagcagctcttagatgacttccacagggtgaccaaagggggctccaccgaggacgccagccagtactactgtgacaagaatgataatggtgacagctacttagtcttgatccgtatcacaccagatgaagatggaaaatttggatttaatcttaagggaggagtggatcaaaagatgcctcttgtggtatcaaggataaacccagagtcacctgcggacacctgcattcctaagctgaacgaaggggatcaaatcgtgttaatcaatggccgggacatctcagaacacacgcatgaccaagtggtgatgttcatcaaagccagccgggagtcccactcacgggagctggccctggtgatcaggaggagagctgtccgctcatttgctgacttcaagtctgaagatgaactgaaccagcttttccccgaagccattttccccatgtgtccggagggtggggacactttggagggatccatggcacagctaaagaagggcctcgaaagcgggacggtgctgatccagtttgagcaactctacagaaaaaagccaggtttggccatcacgtttgcaaagctgcctcaaaatttggacaaaaaccgatataaagatgtgctgccttatgacaccacccgggtattattgcagggaaatgaagattatattaatgcaagttacgtgaacatggaaattcctgctgctaaccttgtgaacaagtacatcgccactcaggggcccctgccgcatacctgtgcacagttttggcaggttgtctgggatcagaagttgtcactcattgtcatgttgacgactctcacagaacgagggcggaccaaatgtcaccagtactggccagatccccccgacgtcatgaaccacggcggctttcacatccagtgtcagtcagaggactgcaccatcgcctatgtgtcccgagaaatgctggtcacaaacacccagaccggggaagaacacacagtgacacatctccagtacgtcgcatggcctgaccacggtatacccgatgactcctccgactttctggaatttgtaaactatgtgaggtctctgagagtggacagcgagcctgtcctagttcactgcagtgctggaataggtcgaaccggtgtgttggtcactatggaaacagccatgtgcctaactgagaggaacctgcccatttacccactggatattgtccgaaaaatgcgagaccagcgcgccatgatggtgcagacatcaagccagtacaagtttgtgtgtgaagcgattcttcgtgtgtatgaagaaggtttagtccaaatgctggatcctagttaagacaactgtgaaaaagttcattcctctttcccaagggcatcctccttgaaagaggaggacagacctctctggaagcagcaagaggaaccagtagctgtgggaaaggaatgggcacctctgaacccaggcactttaaacttctatagaaaagatatcgtgtacataggaactggtgtagataagcatgcaattatggcatcatttaggcctgtatttctatggaaagatacaaaaaggatctcagtttggggcctgtcctaatgccttcttccctaacatcaccacacacacccctgtcggcatcctggagcaattgagaccggacacccacagagctgttgtcctcccagcaacaagatggtgtggttatcttgggtcatttggatgttttgtttgtttctgtgtgtcagactgtaagggctgagctttctgtgcttctaggtggagctggaacaattcagattcacccgccctgatgctaaggaaaccctgacgtatgtactagatggcagggcactgggggtcaggctgaaggctgagcaacacctctctgccctccctccctttgtcccatctcccagcgacttccaatattcatgtttctgagaattgtgtccctcttcagttccctcttggtgcctaacctggattagtaatgtgcattcaggtgaattttcagctgaggctctgagaactggtactctcagtgtgttctggtcatcttgtggcttagttgtagaagcaggtgtgtctcttgcctctgcttgcctcctactgcacactcagcacccaggactggaatcaccgactactgaatctcctacatgtattgctgctacttcaagctcctccacttgaaaccttatgattttccaaggggagatgggacagtgtcatctaaatattccgaatgtttggccttctgagaaaagagcttctagtaattgaaccatgggtttcccagcttctggagggttggccgtgggctgtgtacatgtgtgtgcccaggggtgagtgtttctcaggattcctaacgattcaaattaccgttgagtatatataaagaatcgagtctctgtatggaagaacaaatgtgtgcattcacccccagtcacaatggtctccattgcatttcaaaggagaggatcagactatctgaatataaacacaatctgatgttaatttattctaagaacaccatcattttgattgtcctaaa

SEQ ID NO: 22 - protein, 913 aa, Homo sapiens [sequence omitted]

SEQ ID NO: 23 - DNA, 3087 bp, Mus musculus
  mouse protein tyrosine phosphatase, non- receptor type 3 (PTPN3) cDNA
atggtgaataagccaagggacccccccccccaacctggaatccagggtatctttcatccagctcaggaaacaccaaccggaattcctgggctccagagaactgtaggatgggaatatgaagttaagcagctgttttctgggaagctggccagaaagtacatccctgattccagtgacatttttattgaaaagggcatggtggaggtggtgggttactcggcggccgttatgacctcccggttacgtgcgttgggtggaagaattaacaatacccgaacctctgaattgcccaaagagaaaactcgatccgaggtcatctgcagcatacggtttttagatggcttggtacagacctttaaagttaacaaacaagatttgggccagtcgcttctggatatggcctatggccacctgggtgtgacggagaaggaatacttcggcttgcaacatggcgacgacccagtggactctcctagatggcttgaggcaagcaaacccctcaggaagcagctgaaaggtgagtatgctcttgcttcacttggtcgctgggtgtacggcaaactgtcttctccttatgggggacttaaggtggcgggaaaacctaatctgtttttgaaaaatgttgtaggaggttttccctgtaccctgcattttcgagtaagatattttatacctgatcccaacacactgcagcaggagcaaaccaggcatctatatttcttacaactgaagatggatgtttgcgaaggaaggttaacctgccctctcaactcagcggtggttctagcctcgtacgcagtacaatctcattttggagactttaattcttcaatacatcatccaggctatcttgccgacagccagttcataccagatcaaaatgatgactttttaagcaaggtggagtcactccatgaacagcacagtgggctaaagcagtcggaagccgaatcttgctacatcaacatagctcgaacccttgacttctacggagtggagcttcatggaggcagagatctgcacaacctagatctaatgatcgggattgcgtctgcaggcattgcagtctaccgaaaatacatttgcacaagtttctacccttggaaaaagttcttcatacaccagcggcaaaaacaggaagaaaagattgtggccgtcaggtcttcagaccctgtggccatctctgctgaatccagggaacatatcgtggccttcaacatgctgaattaccgatcttgcaaaaacttgtggaaatcctgcgtcgaacaccattcgttctttcaggcaaaaaagttactacctcaggaaaagaatgttctctctcagtactggactctaggctccaggaaccccaaaaagtctgtaaataaccaatattgcaaaaaggtgattggagggatggtgtggaacccagtcatgaggaggtccttatcagtggaacggctggaaaccaagagcctgccttcccggtcccctcccatcacccccaactggcgaagtcctcggctccggcatgaaatccgcaagccccgccattcctccgcggacaacctggccaatgagatgacctacatcacagaaaccgaggatgtgttctatacctacaagggacccctgtcccccaaagacagcgactctgaagtctcccagaaccacagcccacaccgggagagtttgtccgaaaacaacccagcccaaagctgcctgacccagaagtcatccagttctgtgtctccatcttcgaatgctccaggctcctgctctccagatggagttgaccagcggttcttagaagactaccacaaggtgaccaaaggggggttcgtcgaggacgccagccagtactactgtgataagagtgacgatggagatggctacctagtcctgatccggatcacaccagatgaagagggaagatttggctttaaccttaaggcagatacctgcatgcctaagctgaatgagggggatcagatcgtgttgatcaatggccgggacatctcagagcacactcatgaccaggtggtgatgttcatcaaagcaagccgggagtcccactcgagagaactggccctggtgatcaggagaaaaggaaaagctacctttgtgggccatgagggactggtacctgctagagctgtgcgctcgctggcggagatcagatcagaagatgagctgagtcagcttttcccagaagccatgtttcctgcatgtcctgagggtggggacagtctggagggatccatggagctgctgaagaaaggtctggaaagcggtacagtgttgatccagtttgagcaactctacagaaagaagcccggcttagccgtctcatttgcaaagctgcctcagaatttggataaaaaccgatacaaagatgtgttgccttatgataccacgcgggtattattgcagggaaatgaagattacattaatgccagttatgtgaacatggagatgcccgctgctaaccttgtgaacaagtacattgccactcagggaccgctgccaaacacctgtgcacagttttggcaagttgtctgggatcagaagttgtctcttgttgtcatgctgacgactctcaccgagcgagggcggaccaaatgtcaccagtactggccggatccccctgacatcatggaccatggcatctttcacatccagtgtcagacagaggactgtaccattgcttacgtgtcccgagaaatgctagttacaaacacggagaccggggaagaacacacagtgacccatctccagtatgtggcgtggcctgaccacggcgtacctgatgactcatcagacttcctggagtttgtgaagtatgtgaggtccctgagggtcgacggtgagcctgccctcgttcactgcagtgctggaataggtcggacaggtgttttagtcactatggaaacagccatgtgcttaattgagaggaacctgcctgtttacccactggatattgtccggaaaatgagagaccagcgcgcgatgatggtgcagacgtcaagccaatacaagtttgtgtgtgaagcgatccttcgagtgtacgaagaaggcttagtccagaggctggatcccagttag

SEQ ID NO: 24 - protein, 1028 aa, Mus musculus
  mouse protein tyrosine phosphatase, non- receptor type 3 (PTPN3)
MVNKPRDPPPQPGIQGIFHPAQETPTGIPGLQRTVGWEYEVKQLFSGKLARKYIPDSSDIFIEKGMVEVVGYSAAVMTSRLRALGGRINNTRTSELPKEKTRSEVICSIRFLDGLVQTFKVNKQDLGQSLLDMAYGHLGVTEKEYFGLQHGDDPVDSPRWLEASKPLRKQLKGEYALASLGRWVYGKLSSPYGGLKVAGKPNLFLKNVVGGFPCTLHFRVRYFIPDPNTLQQEQTRHLYFLQLKMDVCEGRLTCPLNSAVVLASYAVQSHFGDFNSSIHHPGYLADSQFIPDQNDDFLSKVESLHEQHSGLKQSEAESCYINIARTLDFYGVELHGGRDLHNLDLMIGIASAGIAVYRKYICTSFYPWKKFFIHQRQKQEEKIVAVRSSDPVAISAESREHIVAFNMLNYRSCKNLWKSCVEHHSFFQAKKLLPQEKNVLSQYWTLGSRNPKKSVNNQYCKKVIGGMVWNPVMRRSLSVERLETKSLPSRSPPITPNWRSPRLRHEIRKPRHSSADNLANEMTYITETEDVFYTYKGPLSPKDSDSEVSQNHSPHRESLSENNPAQSCLTQKSSSSVSPSSNAPGSCSPDGVDQRFLEDYHKVTKGGFVEDASQYYCDKSDDGDGYLVLIRITPDEEGRFGFNLKADTCMPKLNEGDQIVLINGRDISEHTHDQVVMFIKASRESHSRELALVIRRKGKATFVGHEGLVPARAVRSLAEIRSEDELSQLFPEAMFPACPEGGDSLEGSMELLKKGLESGTVLIQFEQLYRKKPGLAVSFAKLPQNLDKNRYKDVLPYDTTRVLLQGNEDYINASYVNMEMPAANLVNKYIATQGPLPNTCAQFWQVVWDQKLSLVVMLTTLTERGRTKCHQYWPDPPDIMDHGIFHIQCQTEDCTIAYVSREMLVTNTETGEEHTVTHLQYVAWPDHGVPDDSSDFLEFVKYVRSLRVDGEPALVHCSAGIGRTGVLVTMETAMCLIERNLPVYPLDIVRKMRDQRAMMVQTSSQYKFVCEAILRVYEEGLVQRLDPS

SEQ ID NO: 25 - DNA, 2607 bp, Homo sapiens
  human protein tyrosine phosphatase, non- receptor type 3 (PTPN3b) splice variant cDNA
atgacctcccggttacgtgcgttgggtggaagaattaataatatacgcacctcggagttacccaaagagaaaactcgatcagaagtcatttgcagcatccactttttagatggcgtggtacagacctttaaagttactaaacaagacactggccaggttcttctggatatggtgcacaaccacctgggtgtgactgaaaaggaatattttggtttacagcatgatgacgactccgtggactctcctagatggctggaagcaagcaaacccatcaggaagcagttaaaaggaggtttcccctgtaccctgcattttcgagtaagattttttatacctgatcccaacacactgcagcaagaacaaaccaggcacttgtatttcttacaactgaagatggatatttgcgaaggaaggttaacctgccctcttaactcagcagtggttctagcgtcctatgccgtacaatctcattttggagactataattcttccatacatcatccaggctatctttccgatagtcactttatacccgatcaaaatgaggactttttaacaaaagtcgaatctctgcatgagcagcacagtgggctaaaacaatcagaagcagaatcctgctatatcaacatagcgcggaccctcgacttctatggagtagaactgcacagtggtagggatctgcacaatttagacctaatgattggaattgcttccgcgggtgttgctgtgtaccgaaaatacatttgcacaagtttctatccttgggtgaacattctcaaaatttctttcaaaaggaaaaagttcttcatacatcagcgacagaaacaggctgaatccagggaacatattgtggccttcaacatgctgaattaccgatcttgcaaaaacttgtggaaatcctgtgttgagcaccatacgttctttcaggcaaagaagctactacctcaggaaaagaatgttctgtctcagtactggactatgggctctcggaacaccaaaaagcgaagtcctcggctccggcacgaaatccgaaagccacgccactcttctgcagataaccttgcaaatgaaatgacctacatcacggaaacggaagatgtattttacacgtacaagggctctctggcccctcaagacagcgattctgaagtttctcagaaccgaagcccgcaccaagagagtttatccgagaacaatccggcacaaagctacctgacccagaagtcatccagttctgtgtctccatcttcaaatgctccaggctcctgctcacctgacggcgttgatcagcagctcttagatgacttccacagggtgaccaaagggggctccaccgaggacgccagccagtactactgtgacaagaatgataatggtgacagctacttagtcttgatccgtatcacaccagatgaagatggaaaatttggatttaatcttaagggaggagtggatcaaaagatgcctcttgtggtatcaaggataaacccagagtcacctgcggacacctgcattcctaagctgaacgaaggggatcaaatcgtgttaatcaatggccgggacatctcagaacacacgcatgaccaagtggtgatgttcatcaaagccagccgggagtcccactcacgggagctggccctggtgatcaggaggagagctgtccgctcatttgctgacttcaagtctgaagatgaactgaaccagcttttccccgaagccattttccccatgtgtccggagggtggggacactttggagggatccatggcacagctaaagaagggcctcgaaagcgggacggtgctgatccagtttgagcaactctacagaaaaaagccaggtttggccatcacgtttgcaaagctgcctcaaaatttggacaaaaaccgatataaagatgtgctgccttatgacaccacccgggtattattgcagggaaatgaagattatattaatgcaagttacgtgaacatggaaattcctgctgctaaccttgtgaacaagtacatcgccactcaggggcccctgccgcatacctgtgcacagttttggcaggttgtctgggatcagaagttgtcactcattgtcatgttgacgactctcacagaacgagggcggaccaaatgtcaccagtactggccagatccccccgacgtcatgaaccacggcggctttcacatccagtgtcagtcagaggactgcaccatcgcctatgtgtcccgagaaatgctggtcacaaacacccagaccggggaagaacacacagtgacacatctccagtacgtcgcatggcctgaccacggtatacccgatgactcctccgactttctggaatttgtaaactatgtgaggtctctgagagtggacagcgagcctgtcctagttcactgcagtgctggaataggtcgaaccggtgtgttggtcactatggaaacagccatgtgcctaactgagaggaacctgcccatttacccactggatattgtccgaaaaatgcgagaccagcgcgccatgatggtgcagacatcaagccagtacaagtttgtgtgtgaagcgattcttcgtgtgtatgaagaaggtttagtccaaatgctggatcctagttaa

SEQ ID NO: 26 - protein, 868 aa, Homo sapiens
  human protein tyrosine phosphatase, non- receptor type 3 (PTPN3b) splice variant
MTSRLRALGGRINNIRTSELPKEKTRSEVICSIHFLDGVVQTFKVTKQDTGQVLLDMVHNHLGVTEKEYFGLQHDDDSVDSPRWLEASKPIRKQLKGGFPCTLHFRVRFFIPDPNTLQQEQTRHLYFLQLKMDICEGRLTCPLNSAVVLASYAVQSHFGDYNSSIHHPGYLSDSHFIPDQNEDFLTKVESLHEQHSGLKQSEAESCYINIARTLDFYGVELHSGRDLHNLDLMIGIASAGVAVYRKYICTSFYPWVNILKISFKRKKFFIHQRQKQAESREHIVAFNMLNYRSCKNLWKSCVEHHTFFQAKKLLPQEKNVLSQYWTMGSRNTKKRSPRLRHEIRKPRHSSADNLANEMTYITETEDVFYTYKGSLAPQDSDSEVSQNRSPHQESLSENNPAQSYLTQKSSSSVSPSSNAPGSCSPDGVDQQLLDDFHRVTKGGSTEDASQYYCDKNDNGDSYLVLIRITPDEDGKFGFNLKGGVDQKMPLVVSRINPESPADTCIPKLNEGDQIVLINGRDISEHTHDQVVMFIKASRESHSRELALVIRRRAVRSFADFKSEDELNQLFPEAIFPMCPEGGDTLEGSMAQLKKGLESGTVLIQFEQLYRKKPGLAITFAKLPQNLDKNRYKDVLPYDTTRVLLQGNEDYINASYVNMEIPAANLVNKYIATQGPLPHTCAQFWQVVWDQKLSLIVMLTTLTERGRTKCHQYWPDPPDVMNHGGFHIQCQSEDCTIAYVSREMLVTNTQTGEEHTVTHLQYVAWPDHGIPDDSSDFLEFVNYVRSLRVDSEPVLVHCSAGIGRTGVLVTMETAMCLTERNLPIYPLDIVRKMRDQRAMMVQTSSQYKFVCEAILRVYEEGLVQMLDPS

SEQ ID NO: 27 - DNA, 861 bp, Homo sapiens
  human dual specificity phosphatase (tyrosine/serine), catalytic domain (DUSP3) cDNA
gccgggcgtgcagggccccgccgccgccatgtcgggctcgttcgagctctcggtgcaggatctcaacgacctgctctcggacggcagcggctgctacagcctcccgagccagccctgcaacgaggtcaccccgcggatctacgtgggcaacgcgtctgtggctcaggacatccccaagctgcagaaactaggcatcacccatgtgctgaacgcggctgagggcaggtccttcatgcacgtcaacaccaatgccaacttctacaaggactccggcatcacatacctgggcatcaaggccaacgacacacaggagttcaacctcagcgcttactttgaaagggctgccgacttcattgaccaggctttggctcaaaagaatggccgggtgctcgtccactgccgggaaggttatagccgctccccaacgctagttatcgcctacctcatgatgcggcagaagatggacgtcaagtctgccctgagcatcgtgaggcagaaccgtgagatcggccccaacgatggcttcctggcccagctctgccagctcaatgacagactagccaaggaggggaagttgaaaccctagggcacccccaccgcctctgctcgagaggtccgtgggggaggccgtgggaaaggtgtccgagctgccatgtttaggaaacacactgtaccctgctcccagcatcacaaggcacttgtctacaagtgtgtcccaacacagtcctgggccactttccccaccctggggagcacataaagaagcttgccaaggggggcgtccttgctccccagttgtcctgtttctgtaacttatgatgtcttttccctgagatgggggctcagagggggaaggcctgt

SEQ ID NO: 28 - protein, 185 aa, Homo sapiens
  human dual specificity phosphatase (tyrosine/serine), catalytic domain (DUSP3)
MSGSFELSVQDLNDLLSDGSGCYSLPSQPCNEVTPRIYVGNASVAQDIPKLQKLGITHVLNAAEGRSFMHVNTNANFYKDSGITYLGIKANDTQEFNLSAYFERAADFIDQALAQKNGRVLVHCREGYSRSPTLVIAYLMMRQKMDVKSALSIVRQNREIGPNDGFLAQLCQLNDRLAKEGKLKP

SEQ ID NO: 29 - DNA, 1196 bp, Mus musculus
  mouse dual specificity phosphatase (tyrosine/serine), catalytic domain (DUSP3) cDNA
ccgctgacccggcttctccccctcgttcgctcctgccctggcgtgcagcgccccgccgccgccatgtccagctcgttcgaactctcggtgcaagatctcaacgacctgctctcggatggcagcggctgctacagcctgccgagccagccctgcaacgaggtcgtcccgagggtctacgtgggcaacgcgtctgtggctcaggacatcacccagctgcagaaactgggcatcacccacgtcctgaatgctgccgagggcaggtccttcatgcacgtcaacaccagtgctagcttctacgaggattctggcatcacctacttgggcatcaaggccaatgatacgcaggagttcaacctcagtgcttactttgaaagggccacagatttcattgaccaggcgctggcccataaaaatggccgggtgcttgtccattgccgcgagggctacagccgctccccaacgctagttatcgcctacctcatgatgcggcagaagatggacgtcaagtctgctctgagtactgtgaggcagaatcgtgagatcggccccaacgatggcttcctggcccaactctgccagctcaatgacagactagccaaggagggcaaggtgaaactctagggtgcccacagcctcttttgcagaggtctgactgggagggccctggcagccatgtttaggaaacacagtatacccactccctgcaccaccagacacgtgcccacatctgtcccactctggtcctcgggggccactccacccttagggagcacatgaagaagctccctaagaagttctgctccttagccatcctttcctgtaatttatgtctctccctgaggtgaggttcaggtttatgtccctgtctgtggcatagatacatctcagtgacccagggtgggagggctatcagggtgcatggcccgggacacgggcactcttcatgacccctcccccacctgggttcttcctgtgtggtccagaaccacgagcctggtaaaggaactatgcaaacacaggccctgacctccccatgtctgttcctggtcctcacagcccgacacgccctgctgaggcagacgaatgacattaagttctgaagcagagtggagatagattagtgactagatttccaaaaagaaggaaaaaaaaggctgcattttaaaattatttccttagaattaaagatactacat

SEQ ID NO: 30 - protein, 185 aa, Mus musculus
  mouse dual specificity phosphatase (tyrosine/serine), catalytic domain (DUSP3)
MSSSFELSVQDLNDLLSDGSGCYSLPSQPCNEVVPRVYVGNASVAQDITQLQKLGITHVLNAAEGRSFMHVNTSASFYEDSGITYLGIKANDTQEFNLSAYFERATDFIDQALAHKNGRVLVHCREGYSRSPTLVIAYLMMRQKMDVKSALSTVRQNREIGPNDGFLAQLCQLNDRLAKEGKVKL

SEQ ID NO: 31 - DNA, 753 bp, Homo sapiens
  human regulator of G-protein signaling 10 (RGS10) cDNA
taccgagctcggatccactagtaacggccgccagtgtgctggaattcgcccttactcactatagggctcgagcggccgcccgggcaggtggattgttggtctgcgtggaacttctcaggtggacaccagagcatggaacacatccacgacagcgatggcagttccagcagcagccaccagagcctcaagagcacagccaaatgggcggcatccctggagaatctgctggaagacccagaaggcgtgaaaagatttagggaatttttaaaaaaggaattcagtgaagaaaatgttttgttttggctagcatgtgaagattttaagaaaatgcaagataagacgcagatgcaggaaaaggcaaaggagatctacatgacctttctgtccagcaaggcctcatcacaggtcaacgtggaggggcagtctcggctcaacgagaagatcctggaagaaccgcaccctctgatgttccagaaactccaggaccagatctttaatctcatgaagtacgacagctacagccgctttcttaagtctgacttgtttttaaaacacaagcgaaccgaggaagaggaagaagatttgcctgatgctcaaactgcagctaaaagagcttccagaatttataacacatgagcccccaaaaagccgggactggcagctttaagaagcaaaggaatttcctctcaggacgtgccgggtttatcattgctttgttatttgtaaggactgaaatgtacaaaacccttcaat

SEQ ID NO: 32 - protein, 167 aa, Homo sapiens
  human regulator of G-protein signaling 10 (RGS10)
MEHIHDSDGSSSSSHQSLKSTAKWAASLENLLEDPEGVKRFREFLKKEFSEENVLFWLACEDFKKMQDKTQMQEKAKEIYMTFLSSKASSQVNVEGQSRLNEKILEEPHPLMFQKLQDQIFNLMKYDSYSRFLKSDLFLKHKRTEEEEEDLPDAQTAAKRASRIYNT

SEQ ID NO: 33 - DNA, 877 bp, Mus musculus
  mouse regulator of G-protein signaling 10 (RGS10) cDNA
gctcttcgggcttagccgccgcgctgcccggctgctccgtcctctggacgcccgcggcgatgttcacccgcgccgtgagccgactgagcaggaagcggccgccgtctgatatccatgacggagatgggagctcaagcagcggccaccagagccttaagagcacagccaagtgggcatcctccctggagaatcttctggaagacccagaaggggtgcagagattcagggagtttctgaagaaggaattcagcgaagagaatgtcttgttttggctagcgtgtgaagatttcaagaaaacggaggacaggaagcagatgcaggaaaaggccaaggagatctacatgaccttcctgtccaataaggcctcttcacaagtcaacgtggaggggcagtctcggctcactgaaaagattctggaagagccacaccctctgatgttccaaaagctccaggaccagatcttcaatctcatgaagtatgacagctacagccgcttcttgaagtctgacttgtttctgaaacccaagcgaactgaggaagaggaagaagagcccccggatgctcagaccgcagctaagcgagcctccagaatttacaacacataagctgagcccttcaccccagcgaaggagagggatggactcttaggactgtacaggctgtcatttctttgttgtgtttgaggactggagtgtgctagaccttccctctggatatgtgtattttattaactgaacagcaacctctgcatgatgctaatcttccattaaaaacaaaagtagctttaaagtgtcagttcacaaaaacacatgagattctgccaatactggacactcagcctttcaatcctgattaaagtgttcgtgaagctacaagc

SEQ ID NO: 34 - protein, 181 aa, Mus musculus
  mouse regulator of G-protein signaling 10 (RGS10)
MFTRAVSRLSRKRPPSDIHDGDGSSSSGHQSLKSTAKWASSLENLLEDPEGVQRFREFLKKEFSEENVLFWLACEDFKKTEDRKQMQEKAKEIYMTFLSNKASSQVNVEGQSRLTEKILEEPHPLMFQKLQDQIFNLMKYDSYSRFLKSDLFLKPKRTEEEEEEPPDAQTAAKRASRIYNT

SEQ ID NO: 35 - protein, 6 aa, Artificial Sequence
  Description of Artificial Sequence hexahistidine (His) affinity tag
HHHHHH

SEQ ID NO: 36 - protein, 200 aa, Artificial Sequence
  Description of Artificial Sequencepoly-Gly flexible linker
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG